(19) United States
(12) Patent Application Publication
(10) Pub. No.: US 2025/0041259 A1
(43) Pub. Date: Feb. 6, 2025

(54) TOPICAL COMPOSITIONS FOR REDUCING SCARRING

(71) Applicant: Omeza Holdings, Inc., Sarasota, FL (US)

(72) Inventors: Lalania Hayes, Sarasota, FL (US); Griscom Bettle, III, Sarasota, FL (US)

(21) Appl. No.: 18/920,409

(22) Filed: Oct. 18, 2024

Related U.S. Application Data
(63) Continuation of application No. PCT/US2023/019060, filed on Apr. 19, 2023.
(60) Provisional application No. 63/332,314, filed on Apr. 19, 2022.

(51) Int. Cl. A61K 31/23; A61K 35/56; A61K 36/899; A61P 17/02

(52) U.S. Cl. CPC A61K 31/23; A61K 35/56; A61K 36/899; A61P 17/02

(57) ABSTRACT

The present disclosure pertains to an anhydrous topical composition for retarding and/or inhibiting scarring of a wound, burn and/or skin condition located on the skin of a subject. Additionally, the topical composition may comprise the following: MCT, monolaurin, cetyl esters, marine oil, vegetable oil having an omega 3 fatty acid content greater than 9 wt %, and/or rice bran wax.

### CROSS REFERENCE TO RELATED APPLICATION

The present application is a continuation of and claims priority to International PCT application No. PCT/US2023/019060, entitled “TOPICAL COMPOSITIONS FOR REDUCING SCARRING”, filed in the United States Patent and Trademark Office on Apr. 19, 2023 by the same inventors, which claims the benefit of and priority to U.S. Provisional Patent Application No. 63/332,314, entitled “TOPICAL COMPOSITIONS FOR REDUCING SCARRING”, filed in the United States Patent and Trademark Office on Apr. 19, 2022 by the same inventors.

Where permitted, the subject matter of each of these applications is incorporated by reference in its entirety.

### FIELD OF THE DISCLOSURE

The present disclosure relates to a topical composition and to the use thereof for promoting wound and burn healing as well as healing from skin conditions associated with broken skin and for reducing scar tissue associated with the healing process.

### BACKGROUND OF THE DISCLOSURE

A wound is a disruption of tissue integrity that is typically associated with a degenerative or traumatic loss of biological substance. Simple wounds include cuts and scrapes to the skin, whilst deeper injuries to the muscle tissue, skeletal system or the inner organs are defined as complicated wounds.

Normal skin consists of collagen fibers arranged in a basket-weave pattern, which contributes to both the strength and elasticity of the dermis. However, wounds disrupt normal skin. Wounds often heal leaving a visible and unsightly scar, and the resulting tissue is usually weaker than the surrounding tissue because the collagen does not heal in its original orientation.

Scarring is caused by excess production of collagen during healing. Collagen is the major structural protein of skin and is responsible for its tensile strength, elasticity, and pliability. It is synthesized in the dermis by fibroblasts. The healing of a wound is a series of complex biological events taking place over an extended period of time. When tissue is cut, the edges of the wound separate and pull apart by the elasticity of the skin. Blood from severed blood vessels fills the cavity of the wound. The blood clots dry, and become hard, forming a scab. The scab shrinks during the first 24 hours, drawing the edges of the wound closer together. Gradually, a grayish, thin membrane extends out from the skin edge and covers the whole wound surface after the scab falls off. The area of the wound is steadily reduced by a process of contraction until there is no raw surface area.

The scar surface area gradually fades until it is paler than the surrounding skin. The process of contraction continues even after the scar is formed as is shown by the gradual shortening of the wound. Some wounds during healing will cross normal skin lines and become depressed below the level of the surrounding skin.

On a microscopic scale, the wound-healing process consists of the development of fibrin which causes the blood clot to contract. White cells arrive at the wound site, and macrophages digest debris present in the wound. Growth of blood capillaries is followed by the inward growth of fibrous tissue migrating from the cells on the wound's margin area. The developing fibrous tissue increases and eventually fills the wound cavity with a network of interlacing threads of collagen that finally arrange themselves in firm bands.

During this process, the surface area of the wound becomes covered by a process of enlargement, flattening, and multiplication of the preexisting epithelial skin cells at the edge of the wound. The epithelial cells divide and spread down into the wound and eventually cause the wound to coalesce to perfect healing.

The wound healing process is a complicated series of events that begins at the moment of injury and can continue for months to years. Specifically, wound healing in adult tissues is a complicated reparative process. For example, the healing process for skin involves the recruitment of a variety of specialized cells to the site of the wound, extracellular matrix and basement membrane deposition, angiogenesis, selective protease activity and re-epithelialization.

There are four distinct phases in the wound healing process. The wound healing process begins immediately after injury. The first stage is the hemostasis phase. This phase includes vasoconstriction lasting for the about the first 5-20 minutes after an injury. The next phase is an inflammatory stage. The inflammatory phase-typically occurs at about 10 minutes after the initial injury, while the cellular response typically starts about 30 minutes after the initial injury. Usually, it lasts two to five days after a wound occurs. During this stage, platelets aggregate to deposit granules, promoting the deposit of fibrin and stimulating the release of growth factors. Leukocytes migrate to the wound site and begin to digest and transport debris away from the wound. During this inflammatory phase, monocytes are also converted to macrophages, which release growth factors for stimulating angiogenesis and the production of fibroblasts.

Next, in the proliferative phase, which typically occurs from two days to three weeks after the wound has occurred, granulation tissue forms and epithelialization begins. Fibroblasts, which are key cell types in this phase, proliferate and synthesize collagen to fill the wound and provide a strong matrix on which epithelial cells grow. As fibroblasts produce collagen, vascularization extends from nearby vessels to supply nutrients to the regenerating tissue. The red loops of blood vessels give the wound a granular appearance, thus the term “granulating” tissue. Epithelialization involves the migration of epithelial cells from the wound surfaces to seal the wound. Epithelial cells are driven by the need to contact cells of like type and are guided by a network of fibrin strands that function as a grid over which these cells migrate. Contractile cells called myofibroblasts appear in wounds, and aid in wound closure. These cells exhibit collagen synthesis and contractility and are common in granulating wounds. It is during the proliferative stage that the extracellular matrix is synthesized in order to provide structural integrity to the wound.

Finally, in the remodeling phase, the final phase of wound healing which can take place from three weeks up to several years from the time of the initial wound occurrence, collagen in the scar undergoes repeated degradation and re-synthesis. During this phase, the tensile strength of the newly formed skin increases. During the remodeling stage the previously constructed and randomly organized matrix is remodeled into an organized structure that is highly cross-linked and aligned to increase mechanical strength.

A scar forms in response to cutaneous injury as part of the natural wound healing process. As the rate of wound healing increases, there is often an associated increase in scar formation. Scarring is a consequence of the healing process in most adult animal and human tissues. Scar tissue is not identical to the tissue which it replaces, as it is usually of inferior functional quality. For example, scars in the skin are less resistant to ultraviolet radiation and sweat glands and hair follicles do not grow back within scar tissue. The types of scars include, but are not limited to, atrophic, hypertrophic and keloidal scars, as well as scar contractures. Atrophic scars are flat and depressed below the surrounding skin as a valley or hole. Hypertrophic scars are elevated scars that remain within the boundaries of the original lesion, and often contain excessive collagen arranged in an abnormal pattern. Keloidal scars are elevated scars that spread beyond the margins of the original wound and invade the surrounding normal skin in a way that is site specific, and often contain whorls of collagen arranged in an abnormal fashion. Scar contractures are scars that cross joints or skin creases at right angles and are prone to developing shortening or contracture. Scar contractures occur when the scar is not fully matured, often tend to be hypertrophic, and are typically disabling and dysfunctional. Scarring can also be ischemic or striae. Ischemic scars result from the local deficiency of blood supply. Striae scars form when skin is stretched rapidly (for instance during pregnancy, significant weight gain or adolescent growth spurts), or when skin is put under tension during the healing process (usually near joints). This type of scar usually improves in appearance after a few years.

The changing patterns of the connective tissue matrix during repair following injury require a delicate balance between synthesis and degradation of collagen and proteoglycans. Under normal circumstances this balance is maintained, while in many diseased states, it is altered, leading to an excessive deposition of collagen, to a loss of functional tissue, or to disfigurement. With hypertrophic scars and keloids, the biosynthetic phase continues longer than necessary to repair the wound. In order to maintain nutrient supply, vascular in-growth occurs, resulting in large, highly vascularized scars which are unsightly and can be disabling. Keloids and hypertrophic scars result in functional and cosmetic deformity. They are a common clinical problem.

While the histological features characterizing hypertrophic scars have been well documented, the underlying pathophysiology is not well known. Hypertrophic scars are a side effect of excessive wound healing. Hypertrophic scars are thick and take the form of a raised scar on the skin as a result of overproduction of cells, collagen, and proteoglycans.

A keloid is a raised scar that exceeds the boundaries of the initial injury (unlike hypertrophic scars which typically stay within the wound boundaries) and is rarely corrected by surgical intervention. Keloids are typically characterized as tumors consisting of highly hyperplastic masses that occur in the dermis and adjacent subcutaneous tissue in susceptible individuals, most commonly following trauma. Keloids may grow into a firm lump that is many times larger than the original scar and are typically fibrotic growths that contain a collection of atypical fibroblasts and an increased abundance of extracellular matrix components, especially collagen.

Keloids are often more severe than hypertrophic scars, since they tend to invade normal adjacent tissue, while hypertrophic scars tend to remain confined within the original scar border.

Although commonly benign, hypertrophic scars and keloids often cause discomfort, pain, pruritus, physical disfigurement, and impaired quality of life. Moreover, once scarring has occurred, it cannot be reversed, although considerable shrinking or reduction of the scarring may occur. Typically, scars do not tan in sunlight, nor do they produce hair or sweat. These characteristics are evidence that the skin has failed to return to its full function.

Various approaches have been tried to enhance wound healing and reduce scar formation, such as the application of silicon sheets to the surface of the skin to reduce raised scar formation. Some topical creams and gels are also sold as a remedy for scars with claims of improving the scar's appearance. However, many of these approaches fail to provide the claimed solution. For example, topical creams promoted for scar reduction have not been shown to have efficacy when tested in blinded trials, as they generally do not address the collagen network that reforms following a wound. Most of the scar reduction products contain silicone in a sheet or gel format, and onion extracts (Mederma Skin Care products). It usually takes over 3 months to see some effect, because these products do not contain effective active ingredients such as any form of collagenase which targets the cause of scar formation.

Other attempts to treat hypertrophic scars and keloids include surgery, mechanical pressure, steroids, x-ray irradiation and cryotherapy. There are many disadvantages associated with each of these methods. Surgical removal of the scar tissues is often incomplete and can result in further development of hypertrophic scars and keloids at the incision and suture points. Steroid treatments are unpredictable and often result in depigmentation of the skin. X-ray therapy is the only predictable effective treatment to date; however, because of its potential for causing cancer, it is not generally recommended or accepted. The most common approach to controlling scar, and in particular excessive scar formation, is to apply pressure, which appears to be somewhat effective in many instances. This treatment has limited application, generally based on the size and location of the scar tissue on the body. Other commonly used treatments are application of Vitamin E and corticosteroids.

Unfortunately, these methods have drawbacks and are unsatisfactory to the public. According to market research by The Mattson Jack Group, of the total 42 million surgical procedures in the United States in 2004, 34 percent of patients were at considerable risk of compromised healing. This research showed that there was low satisfaction with current therapies available on the market, and a high interest in remedies that accelerate surgical healing. Thus, there is a clear market need to provide effective medication to promote the healing of wounds. Specifically, it is often desirable to increase the rate of healing in the case of acute wounds (such as penetrative injuries, burns, nerve damage or even wounds resulting from elective surgery), chronic wounds (such as diabetic, venous and decubitus ulceration) or for generally healing-compromised individuals (such as the elderly or diabetic individuals). In all these examples, wounds can severely influence an individual's quality of life, or even result in death. For example, bacterial infection of a wound site can impede the healing process, and lead to life threatening complications. Thus, it is desirable to increase the effectiveness of healing as much as is clinically possible.

The present disclosure provides a solution to the unfulfilled need of the public as it provides a composition and method for reducing and/or preventing the appearance of scar tissue such as caused by surgical, acne, burns, chronic wounds, and trauma-induced scarring.

### SUMMARY OF THE DISCLOSURE

The present disclosure provides, in an embodiment, a composition comprising the following ingredients: MCT, monolaurin, cetyl esters, marine oil, vegetable oils having an omega 3 fatty acid content greater than 9 wt %, such as hemp oil, and rice bran wax. The composition is a topical composition. It is also anhydrous. In an embodiment, it has a penetration factor greater than or equal to 1. In an embodiment, the penetration factor ranges from 1 to about 11. In addition, in an embodiment, the sum of the omega 3 fatty acid content present in the composition described herein ranges from about 0.5 wt % to about 8 wt %. In another embodiment, the weight ratio of the sum of the weights of (ALA+SDA)/sum of the weights of (EPA and DHA) (hereinafter the “PUFA ratio”) ranges from about 0.5 to about 2.0, wherein ALA is α-linolenic acid, SDA is stearidonic acid, EPA is eicosapentaenoic acid, and DHA is docosahexaenoic acid. In an embodiment, the composition has at least two of the following characteristics: the penetration factor ranging from 1 to about 11, the sum of the omega 3 fatty acid content present in the composition ranging from about 0.5 wt % to about 8 wt %, and the PUFA ratio ranging from about 0.5 to about 2.0, and in another embodiment, the composition of the present disclosure has all three of these characteristics. In an embodiment, coconut oil is present in the composition. In another embodiment, ascorbyl palmitate is present in the composition. In a further embodiment, colloidal oatmeal is present in the composition. In an embodiment, the composition has a viscosity ranging from about 50,000 cp to about 400,000 cp at 75° F. In a further embodiment, the composition has a specific gravity ranging from about 0.83 to about 0.92 at 75° F. and 14.7 psig, and in a further embodiment, the composition possesses both of the aforementioned specific gravity and viscosity. Moreover, in another embodiment, when coconut oil is present in the composition, the sum of the weight % of (MCT+coconut oil)/sum of the weight percentage of (monolaurin+cetyl esters+rice bran wax) ranges from about 1 to about 5. In an embodiment, MCT is present in the composition in an amount ranging from about 40 wt % to about 75 wt %, monolaurin is present in an amount ranging from about 7 wt % to about 14 wt %; cetyl esters are present in an amount ranging from about 4 wt % to about 12 wt %; marine oil is present in an amount ranging from about 0.5 wt % to about 8 wt %; vegetable oil having an omega 3 fatty acid content greater than 9 wt %, such as hemp oil, is present in an amount ranging from about 0.5 wt % to about 8 wt %; rice bran wax is present in an amount ranging from about 2 wt % to about 12 wt %; wherein the sum of the above-identified ingredients in the composition is no greater than 100 wt %, and wherein the sum of the weight percentages of all of the ingredients in the composition adds to 100 wt %. In an embodiment, the sum of the aforesaid ingredients ranges from about 90% to about 100 wt % (but no greater than 100%) of the composition. in another embodiment, MCT is present in an amount ranging from about 60 wt % to about 75 wt % of the composition. In an embodiment, the compositions described herein are collagen-free. In an embodiment, when coconut oil is present, it is present in an amount ranging from about 0.1 wt % to about 2 wt %. When colloidal oatmeal is present, in an embodiment, it is present in an amount ranging from about 0.007 wt % to about 5 wt %. Further, in an embodiment, when ascorbyl is present, ascorbyl palmitate is present in an amount ranging from about 0.1 wt % to about 3 wt %,

The composition described herein is useful for treating and reducing the appearance of scar tissue and for promoting healing of wounds, burns or skin conditions. Methods of the present disclosure result in the reduction of the unsightly appearance of scar tissue caused by wounds or burn or skin conditions. The method comprises the step of topically administering a therapeutically effective amount of the composition of the present disclosure to the subject seeking to reduce the appearance of scar tissue by applying topically the composition to the locus of the wound or burn or skin condition at the time of the closure of the skin during the healing process and the disappearance of the raw tissue.

### BRIEF DESCRIPTION OF THE DRAWING

The objects, features and advantages will become apparent to one of ordinary skill in the art, in view of the following detailed description taken in combination with the attached drawings, in which.

FIG. 1 is a graphical representation of a cooling curve of K7p of Example 1, showing the eutectic points of various components therein indicating the precipitation of components therein;

FIG. 2 is a graphical representation of the composition in Example 2 of the cooling rate of K7p at two different mixing rates during the preparation thereof;

FIG. 3 is a graphical representation of a cooling curve of CS1d, Counter Example 1, showing two distinct exotherms;

FIG. 4 is a cooling curve of K7r described in Example 3 showing four distinct freezing exotherms;

FIG. 5 is the same cooling curve as FIG. 4 with respect to K7r of Example 3, showing three of the four freezing exotherms;

FIG. 6 is a schematic representation of the nano-liquid-capsule of the present disclosure after the preparation of the composition (top FIGURE) and the inversion that takes place when the composition is topically applied to the surface of the skin;

FIG. 7 is a graphical representation showing the effect of increasing MCT and monolaurin concentration on the aged specific gravity of the K7p composition;

FIG. 8 is a graphical representation showing the effect of increasing concentration of monolaurin and cetyl esters on the aged specific gravity of the K7p composition;

FIG. 9 is a graphical representation of the effect of increasing concentration of cetyl esters on viscosity and aged specific gravity of the K7p composition;

FIG. 10 is a graphical representation of the cooling curve for the K7s composition.

FIG. 11 is a graphical representation of the change of slope at different points in the cooling curve in FIG. 10 for K7s;

FIG. 12 is a graphical representation of Constant Temperature Freezing in the cooling curve of K7s depicted in FIG. 10;

FIG. 13 is a cooling curve for K7s, highlighting the high temperature transitions;

FIG. 14 is a graphical representation depicting the change in viscosity in the preparation of K7s;

FIG. 15 is a graphical representation that plots K7s viscosity versus specific gravity;

FIG. 16 schematically depicts the noncapsular, layered structure of K7s after it is prepared;

FIG. 17 schematically depicts the inversion of the nanocapsular structure after it is applied to the skin;

FIG. 18 is a photo of a burn on the back of an arm of a female before (left side) and after treatment with K7s (right side);

FIG. 19 is a representation of how saturated triglycerides>C10:0 crystals bond to pre-existing rice bran wax crystals.

FIG. 20 is a graphical representation of K7LH5 cooling curve;

FIG. 21 is a graphical representation of K7LH5 cooling curve with stirring of K7LH5;

FIG. 22 is a graphical representation of K7LH5 quiescent cooling after reheating;

FIG. 23 is a graphical representation of a cooling curve of K7LH5 illustrating the effect of lowering the temperature on tripalmitin present in K7HL5;

FIG. 24 are microscopic photographs. FIG. 24(a) is a microscopic photograph of a nano-liquid capsule, and (b) is a photograph of the microscopic photograph of (a) magnified by a factor of 15.

### DETAILED DESCRIPTION

In an embodiment, the present disclosure may comprise a composition comprised of MCT, monolaurin, cetyl esters, marine oil, vegetable oils having an omega 3 fatty acid content greater than 9 wt %, such as hemp oil, and rice bran wax. In an embodiment, at least one of the following ingredients is present: coconut oil, colloidal oatmeal and ascorbyl palmitate, while in another embodiment, at least two of these three ingredients are additionally present, while in a still further embodiment, the composition additionally comprises coconut oil, colloidal oatmeal and ascorbyl palmitate. The compositions described herein may be anhydrous and/or are homogenous to the naked eye on a macroscopic scale. Further, in an embodiment, they do not exhibit the rancid, fish odor found in fish oil. Further when applied to the skin of a person, the person's skin is moisturized. Moreover, when applied to the skin in treating wounds, burns, or skin conditions, there is either minimal scarring, no visible discernment of scarring with the naked eye or there is an inappreciable amount of scarring.

One of the components of the disclosure described herein is marine oil. Marine oil contains, among other components, omega 3-fatty acids. As defined herein, “marine oil” refers to the oil extracted from fish carcasses or crustaceans or any other marine life. Examples of marine oil where the useful oil containing omega-3 fatty acids include salmon, tuna, swordfish, halibut, tilefish, cod fish (including cod liver oil), anchovies and sardines. Cold-pressed fish oil and heat-treated fish oil are subsumed under the term “marine oil.” Examples of crustaceans from which the oil can be used include krill, a crustacean in the Antarctic (the source of krill oil) and the New Zealand green lipped mussel (also known as Perna canaliculus). In addition, useful oil is derived from marine vegetation, such as Marine algae, and phytoplankton can also be used. All of these oils are a source of omega-3 fatty acids. The term marine oil, as used herein, refers to the marine sources containing omega 3-fatty acids.

The richest dietary source of very long-chain omega-3 polyunsaturated fatty acids (PUFA) come from marine oil, such as fish oil and algae oil. Fatty acids are the building blocks of dietary fats and are stored substantially in the form of triglycerides. The body cannot, however, produce polyunsaturated fatty acids and must obtain them from food sources or from supplements. Nutritionally important omega-3 fatty acids include α-linolenic acid (ALA) (18 carbon unsaturated fatty acid), stearidonic acid (SDA) (18 carbon unsaturated fat, C18:4), eicosapentaenoic acid (EPA) (20 carbon unsaturated fat, C20:5), and docosahexaenoic acid (DHA) (22 carbon unsaturated fatty acid, C22:6).

ALA is found in, e.g., walnuts, some types of beans, hempseed oil and olive oils. EPA, SDA, and DHA are found in fish, including fish oil and algae and supplements. All of these omega3 fatty acids are polyunsaturated fatty acids (PUFA). The omega-3 fatty acids are essential to life at any stage, even before birth. They are essential building blocks of the membrane of every cell in the body and their presence are a necessity for maintaining an adequate cell membrane. They also contribute to the regulation of most biological functions. As described herein, these omega-3 fatty acids are useful in treating wounds, burns and skin conditions.

The terms “algal oil” and “algae oil” are synonymous and as used herein, these terms refer to oil made from certain marine or fresh water or farm-raised algae, such as algae grown in greenhouses or algae grown from any other source. Industrial sources of algae typically come from open recirculating ponds or from glass-enclosed growing containers (similar to greenhouses but are often vertical to better capture light). Open ponds are cheaper but are subject to contamination from wild algae. Glass-enclosed growing areas are used with specialty, high-efficiency algal strains. Useful oil is derived from marine vegetation, such as Marine algae, farm-raised algae, glass-raised algae and phytoplankton. As defined herein, algal oil are subsumed under the term “marine oil.”

All of these fish oils and algae oil are sources of omega3 fatty acids. These oils are extracted from the livers of the fish or from algae using techniques known in the art.

For example, cod liver oil comes from cod fish, e.g., Atlantic cod (Gadus morhua) or from Pacific cod (Gadus microcephalus). Both Atlantic and Pacific cod liver oils are acceptable as marine oil, but Pacific cod liver oil is preferred because the Bering Sea water is pristine with very little heavy metals. Pacific cod liver oil is obtained from wild, line-caught, adult cod. The immediately eviscerated liver, either from Pacific cod liver oil or Atlantic cod liver oil, is frozen on board to retain its nutrients. Frozen livers are transferred to a shore-side processing plant, rendered, and pressed into commercial cod liver oil with almost no odor.

The term “medium-chain triglyceride(s) of C8/C10 fatty acid”, “medium-chain triglyceride(s) of C8/C10 fatty acids”, “medium-chain triglycerides of a mixture of C8 and C10 fatty acids”, “C8/C10 triglycerides”, or “C8/C10 MCT”, “MCT oil”, or “MCT” are interchangeable and refer to a triglyceride of C8 fatty acids, a triglyceride of C10 fatty acids or a triglyceride of a C12 fatty acid or a mixture thereof. MCT is commercially available, such as, for example, from PG Chemicals, Cincinnati, OH. In addition, the term C8 triglyceride refers to a triglyceride of a C8 fatty acid, and the term C10 triglyceride refers to a triglyceride of a C10 fatty acid. In an embodiment, it is a mixture of triglycerides of C8 and C10 fatty acids, wherein the amount by weight of triglycerides of C8 fatty acids present is greater than the amount by weight of triglycerides C10 present. It is to be understood that a MCT comprises additional triglycerides besides C8 and C10 triglycerides. MCT comprises a triglyceride backbone having attached thereto three fatty acid chains that are generally from about C6 to C12 in length, although shorter or longer chains may be included within the term in differing contexts, as understood by those having skill in the art; but these longer and shorter chains are present in negligible amounts, for example, usually less than 3% by weight. The three medium chain fatty acids that are attached to the triglyceride backbone of the MCT may be, but need not be, identical. The medium chain fatty acids can be either saturated or unsaturated but are preferably saturated. Examples of medium chain fatty acids that comprise the medium chain triglycerides of the present disclosure include C6 (caproic fatty acid), C8 (caprylic fatty acid), C10 (capric fatty acid), and C12 (lauric fatty acid), as well as mixtures thereof. In an embodiment, the MCTs comprise a mixture of from about 60% C8 triglyceride and about 40% C10 triglyceride to a mixture of about 70% C8 triglyceride and about 30% triglyceride C10. In another embodiment, it comprises a mixture of about 51% C8 triglycerides and about 49% C10 triglycerides to about 70% C8 triglycerides to about 30% C10 triglycerides by weight; in another embodiment, it comprises 55% C8 triglycerides and about 45% C10 triglycerides to about 65% C8 triglycerides to about 35% C10 triglycerides by weight. Further, as indicated hereinabove, the MCTs of the present disclosure may include minor amounts of triglycerides of short or long chain fatty acids, such as C6 or C4 fatty acids or C12 or C14 or C16 fatty acids, but the short or long fatty acids are present in minor amounts, e.g., less than about 3% by weight. In another embodiment, the MCT contains no triglyceride of a C12 fatty acid. In an embodiment, MCT is a Capric/Caprylic Triglyceride.

The medium-chain triglyceride(s) of C8/C10 fatty acids (MCT) are prepared by chemical techniques known in the art by separating the naturally occurring fatty acids by chain length, standardizing the blend of C8 and C10 and then esterifying the blended fatty acid with glycerol to make MCT.

The term “vegetable oil having an omega3 fatty acid content greater than 9 wt %” refers to vegetable oils having more than 9 wt % omega3 fatty acids. The compositions of many vegetable oils meeting this criterion are known. For example, canola oil has an alpha-linolenic acid content of 9.1 wt %. Flaxseed oil has an alpha-linolenic acid content of 53 wt %, and hempseed oil has an alpha-linolenic acid content of 22 wt %.

The terms “hempseed oil” and “hemp oil” as used herein, are synonymous. Hemp oil is prepared by pressing hemp seeds, especially hemp seeds that are cold pressed. Unrefined hemp oil is dark to clear light green in color with a nutty flavor. However, in an embodiment, the hemp oil used herein is refined so that it is substantially free of tetrahydrocannabinol. It is manufactured from varieties of Cannabis sativa that are substantially free of tetrahydrocannabinol (THC). In the manufacturing process, THC is removed prior to pressing of the seed oil. In an embodiment, the hemp oil contains at most 1% by weight of THC and in another embodiment, less than or equal to 0.1% by weight THC, and in still further embodiment less than 0.01% by weight, if any, of THC. In another embodiment, the THC is not detectable, for example, less than 10 ppm. Hemp oil contains gamma-linolenic acid (GLA), which is an omega-6 fatty acid, as well as alpha-linolenic acid (ALA), which is an omega3 fatty acid. Hemp oil also contains about 14% oleic acid (C18:1), an omega-9 oil.

Monolaurin, as used herein, is also known as glycerol monolaurate, glyceryl laurate or 1-Lauroyl-glycerol. It is a monoglyceride. It is the monoester formed from glycerol and lauric acid. Its chemical formula is C₁₅H₃₀O₄. Monolaurin is a nonionic surfactant.

The term cetyl esters, as defined herein, are unbranched esters formed from cetyl alcohol and a C14, C16 or C18 fatty acid. The fatty acids may be saturated or unsaturated. As used herein, cetyl ester, either in the plural or singular, unless indicated to the contrary, refers to esters of C14, C16, or C18 fatty acid or mixtures thereof and cetyl alcohol. Cetyl Esters is a synthetic wax that has similar composition and chemical properties to a natural wax which is found in sperm whales. The esters that are found in Cetyl Esters include cetyl palmitate, cetyl stearate, myristyl myristate, myristyl stearate, cetyl myristate, and stearyl stearate. In an embodiment, cetyl ester is Cetyl Ester NF, CAS 540-10-3, 2598-99-4, EINECS 208-736, 220-000-6, typically sourced from Rita Corporation, Crystal Lake, IL. Each individual cetyl ester freezes at slightly different temperatures. Longer chain esters freeze at higher temperatures than shorter chain length esters.

The term “oils,” when used alone, refers to the combination of marine oil, C8/C10 triglyceride, and/vegetable oil, and mixtures thereof.

The terms “non-coconut vegetable oil,” or “vegetable oil” are used interchangeably and are synonymous and is distinct from “vegetable oil having an omega 3 fatty acid content greater than 9 wt %”, as used herein, and coconut oil. The term refers to vegetable oils containing no or less than or equal to 9.0 weight % of omega3 fatty acids and does not contain coconut oil. Examples include, corn oil, cottonseed oil, grapeseed oil, olive oil, palm oil, peanut oil, soybean oil, sunflower oil, cottonseed oil, and the like.

As used herein, the term “coconut oil” is a vegetable oil and is generic term that includes crude (virgin) coconut oil and coconut oil that has been refined. Coconut oil is the raw minimally processed oil from coconuts, and as used herein, is “crude coconut oil.” Refined coconut oil is coconut oil that has been refined, bleached, and deodorized, and is referred to herein as refined or RBD coconut oil.” Refined coconut oil has a higher melting point than crude coconut oil. Unless indicated to the contrary, the term “coconut oil” includes both crude coconut oil and refined coconut oil.

As used herein, the terms “palm olein” is synonymous with the term “palm oil” and both are used interchangeably. The term “palm oil” is the liquid portion which is separated from the semi-solid palm oil by fractionation. As used herein, the term includes red palm olein, and super red palm olein. The liquid portion is sold as cooking oils and the solid portion is known as “palm stearin.” When palm olein is fractionated again to get a more liquid fraction, such as by chilling and removing the solid fraction of C18:0 (saturated C18 fatty acids) and some C16:0 (saturated C16 fatty acids), it is known as “super palm olein” or “CP6” (Cloud Point 6° C., meaning the fractionation took place at 6° C.). Palm super olein is capable of withstanding colder temperature in comparison with palm olein after which they turn into solid. Palm olein is commonly used as cooking oil in the tropical countries. But the problem in temperate climate countries is that due to cold weather, palm olein tends to get cloudy and crystallize. To overcome this problem, palm olein is blended with more unsaturated vegetable oils. This blended form can be used in a wide range of climates and has a better cold stability. These blends are also cheaper than non-blended forms. The vegetable oils from rice bran, groundnuts and rapeseed are blended with palm olein to get a superior form in terms of quality and stability.

Palm oil, including red palm oil, is obtained from the endocarp of the palm fruit (the soft flesh); palm kernel oil is obtained from the seed (palm kernel oil). The oils are very much different. Red palm oil has the highest level of antioxidants of any seed crop. The carotenoids (the beta-carotene fraction is a Vitamin A precursor), tocopherols and tocotrienols (Vitamin E) present therein give red palm oil its distinctive color. The carotenoids are bright red; the tocopherols and tocotrienols are yellow; together they make red palm oil orange/red colored. These highly colored compounds are not readily absorbed by the skin and stain the skin surface and any clothing or bedding surface with which it comes in contact. When palm oil is refined, the carotenoids (primarily beta-carotene and lycopene) are removed and remaining palm oil which is RBD (refined, bleached, deodorized) palm oil is yellow colored because Vitamin E remains, and the carotenoids are removed.

It is understood that the terms palm oil and palm olein includes the various types of palm oil, including, but not limited to, red palm oil or red palm olein, RBD palm oil, red palm concentrate, super palm olein, including super red palm olein, squalene/Vitamin E (from red palm oil) and the like.

In an embodiment, the palm oil, the red palm olein, and the super red palm olein and RBD palm oil contains a low amount of C18 fatty acid. By low amount, it means less than 5% by weight.

Colloidal oatmeal is an optional ingredient that is present in the formulations described herein. Colloidal oatmeal is made by grinding oat grain or Avena sativa into a fine powder. It is commercially available. It is an emollient and has been categorized by the FDA in 2003 as a skin protectant if the concentration is greater than 0.007 wt % (there is no regulatory upper limit). It also has anti-inflammatory and antioxidant properties.

As defined herein, the term “free fatty acid” refers to any added FFA, as defined herein as well as the linear carboxylic acid that is formed after the triglyceride is hydrolyzed into free fatty acid and glycerin or into free fatty acid and a monoglyceride or diglyceride. Industrial hydrolysis is complete (i.e., free fatty acid and glycerin). Enzymatic hydrolysis (e.g., in the body) is incomplete (free fatty acid and a glyceryl monoester, like monolaurin or a diglyceride).

The term “coconut FFA,” is a short-hand notation for “free fatty acid” of coconut oil and is a type of free fatty acid. It is a composition comprising predominantly of the fatty acids, caprylic acid C-8:0 (8%), capric acid, C-10:0, (7%), lauric acid C-12:0, (49%), myristic acid C-14:0 (8%), palmitic acid C-16:0 (8%), stearic acid C-18:0 (2%), oleic acid C-18:1 (6%) and 2% of C-18:2 linoleic acid. As used herein, caprylic acid is not coconut FFA or FFA, and is distinct therefrom. Unless indicated to the contrary, the term “caprylic acid” refers to a C8 saturated fatty acid of the formula CH₃(CH₂)₆COOH. Although it may be a component of coconut FFA, when the term “caprylic acid” is used, it is understood to be a compound of the formula given hereinabove, separate, and distinct from the term “coconut FFA,” and “FFA,” as used herein.

The term “anhydrous,” as used herein refers to the water content of the composition of the present disclosure. As defined herein, the water content of the present composition refers to free water, that is, water not chemically bound to a substrate. As defined, the composition contains less than 5% by weight of free water or any range therein. A composition of the present disclosure may have a water content of less than 5, 4.5, 4, 3.5, 3, 2.5, 2, 1.5, 1, 0.5%, or any range therebetween, by weight of the composition. Water content may be measured by methods known to those of skill in the art, such as, but not limited to, Karl Fischer titration.

As defined herein the term “homogenous,” as it relates to a composition described herein means that the components in the composition are substantially visually uniformly distributed throughout the composition at room temperature and following freeze/thaw abuse.

The term “eutectic composition” is a mixture of chemical compounds or elements that has a single chemical composition that melts and solidifies at a temperature that is lower than the melting points of the separate chemical compound constituents that makes up the eutectic composition or any other mixture of them. A composition comprising a eutectic is known as the eutectic composition and its melting temperature is known as the eutectic temperature.

The term “melting point,” as used herein, is used in three senses. It has the usual meaning, in one sense, i.e., the temperature at which a solid melts and forms a liquid. However, with many of the oily compositions described herein, it is difficult to determine or measure the specific melting point. In those cases, one of the following definitions is applicable, dependent upon the specific compositions. In some cases, such as, for example, for oily compositions comprised of hemp oil in the absence of fish collagen, the melting point is defined as the temperature when the oily composition turns cloudy as the temperature is lowered. In other instances, it is defined as the temperature when there is a visible change in viscosity when the temperature is lowered. All three definitions are potentially applicable, but the term melting point with respect to the compositions discussed herein, for purposes of this application, is the lowest temperature in which the composition either melts, as in the traditional definition, or turns cloudy or where there is a visible change in viscosity. The change in viscosity can be subtle, as in the change in slope of the cooling curve before and after the eutectic temperature.

As used herein, the term “collagen” refers to fish collagen or collagen from any other source that has been hydrolyzed.

The present disclosure refers to collagen-free compositions. The term “collagen-free compositions” refers to compositions containing no collagen or less than or equal to 2.0 wt % collagen in the composition. As used herein, the terms non-collagen containing composition or collagen-free composition are synonymous and are used interchangeably. Hydrolyzed collagen is water-soluble, not oil soluble. However, small amounts of collagen can be incorporated into gels and can be mechanically stabilized to appear homogenous. Low amounts of suspended collagen can be advantageous, for example in very gentle facial scrubs or in first aid antiseptics.

As defined herein, there are various ranges of numbers or ratios provided herein. It is to be understood that the ranges include not only the endpoints (plus or minus 5% when modified by the term about), but also all of the integral numbers and fractions therebetween, Thus, for example, if the range is defined as ranging from 5 wt % to 10 wt %, it is understood that the values include the endpoints 5 wt % and 10 wt %, but also all the integers and fractional numbers and real numbers therebetween and each one of those values are described herein, for purposes of the teaching of the disclosure described herein.

The term “TEWL” refers to Trans Epidermal Water Loss, which is the amount of water that passively evaporates through the skin to the external environment due to a water vapor pressure gradient on both sides of the skin barrier.

As used herein, the abbreviation “RBW” refers to rice bran wax. Moreover, the abbreviation “NLC” refers to nano-liquid capsule.

Unless indicated to the contrary, the terms “compositions” and “formulations” alone or in combination with other terms, such as for example, topical, are synonymous and can be used interchangeably.

Unless indicated to the contrary, the terms “drugs” and “medicament” are synonymous.

Treatment” or “treating,” as used herein refers to complete elimination as well as to any clinically or quantitatively measurable healing or alleviation of the symptoms of the wound or burn or skin condition.

A “therapeutically effective amount” means the amount of a composition that, when administered to a subject for treating a wound, burn or skin condition is sufficient to effect a desirable treatment for the wound, burn or skin condition. The “therapeutically effective amount” will vary depending on the particular composition, the condition being treated, and its type and severity, and the age, weight, and the like, of the subject to be treated. The actual amount which comprises the “effective amount” will vary depending on several conditions including, but not limited to, the particular disorder being treated, the severity of the disorder, the size and health of the patient, and the route of administration. A skilled medical practitioner can readily determine the appropriate amount using methods known in the medical arts.

The term “scarring inhibiting effective amount” means the amount of a composition that, when administered to a wound, burn or skin condition on a subject where the wound, burn, or skin condition is fully granulated on the skin, inhibits or retards scarring. The “scarring inhibiting effective amount” will vary depending on the particular composition, the condition and its type and severity, and the age, weight, and the like with respect to the subject being treated. The actual amount which comprises the “effective amount” will also vary depending on several conditions including, but not limited to, the particular disorder being treated, the severity of the disorder, the size and health of the patient, and the route of administration. A skilled medical practitioner can readily determine the appropriate amount using methods known in the medical arts. The scarring inhibiting effective amount is the cross product of dose times frequency of application.

As used herein, the terms that list the number of carbon atoms followed by a colon and then a number, like C18:0, refer to the number of carbon atoms and the number of carbon-carbon double bonds in a compound, such as a triglyceride, in the topical composition. Triglycerides are esters formed from glycerol and three fatty acid groups. The number after the “C” refers to the number of carbon atoms in the fatty acid and the number after the colon refers to the number of carbon-carbon double bonds in the fatty acid. Thus, for example, C18:0 refers to the triglyceride wherein the fatty acid attached to the glycerol moiety contains 18 carbon atoms and has no carbon-carbon double bonds, while the term C18:1 refers to the triglyceride wherein the fatty acid attached to the glycerol contains 18 carbon atoms and has one carbon-carbon double bond, while the term C18:2 refers to the triglyceride wherein the fatty acid attached to the glycerol moiety contains 18 carbon atoms and has two carbon-carbon double bonds.

“Patient” or “subject” refers to animals, and can include any mammal, such as humans, rats, mice, cats, dogs, goats, sheep, horses, monkeys, apes, rabbits, cattle, etc. The mammalian subject can be in any stage of development including adults, children, infants, and neonates.

Unless indicated to the contrary, percentages are by weight and ratios are weight ratios.

The term “about,” as used herein, when used before a number or numerical ranges, in an embodiment herein refers to an amount±5% of that value of the number or range. For example, when referring to a range of about 9 to about 11, it is understood to range from 8.65-11.55.

As used herein, the singular forms “a,” “an” and “the” include plural referents unless the context clearly dictates otherwise.

Furthermore, as used herein, the terms “components” and “ingredients” are synonymous and are used interchangeably.

The term “topical” refers to administration or delivery of the composition described herein by application of the composition to a surface or body part. For example, a composition can be topically administered by applying it to the skin, to the surface of a wound or burn.

Unless indicated to the contrary, all percentages herein refer to weight %.

The term “overnight abuse test” or “temperature abuse test” refers to a test to determine whether the product will separate during distribution and storage or not. The test involves putting about 28 g of test product into a sealed ˜30 ml clear glass jar. The sealed jar is placed in a 40° C. incubator for 15 hours and then the top and bottom of the jar is examined for visual leakage. The assessment is done immediately after the jar is retrieved from the incubator as bottom leakage often congeals once returned to ambient temperature. Top leakage is infrequent but can result from shear induced abuse.

Unless indicated to the contrary, the singular includes the plural and vice versa.

An embodiment of the present disclosure is directed to an anhydrous and topical composition comprising the following ingredients: MCT, monolaurin, cetyl esters, marine oil, vegetable oil having an omega3 fatty acid content greater than 9 wt %, such as hemp oil, and rice bran wax. In an embodiment, the composition of the present disclosure may additionally comprise coconut oil, ascorbyl palmitate and/or colloidal oatmeal, while in another embodiment, the composition may additionally comprise two of the following ingredients: coconut oil, ascorbyl palmitate and/or colloidal oatmeal, and in another embodiment, the composition comprises all three. In an embodiment, it has a specific gravity ranging from about 0.87 to about 0.92 at 75° F. and 1 atm. In an embodiment, it has a viscosity at about 75° F. of about 100,000 cp to about 300,000 cp.

In an embodiment, MCT is present in the composition in an amount ranging from about 40 wt % to 75 wt %; monolaurin is present in an amount ranging from about 7 wt % to about 14 wt %; cetyl esters are present in an amount ranging from about 4 wt % to 12 wt %; coconut oil is present in an amount ranging from about 0.1 wt % to about 2 wt %; marine oil is present in an amount ranging from about 0.5 wt % to about 8 wt %; vegetable oil having an omega3 fatty acid content greater than 9 wt %, such as hemp oil, is present in an amount ranging from about 0.5 wt % to about 8 wt %; and rice bran wax is present in an amount ranging from about 2 wt % to about 12 wt %. When coconut oil is present, it is present in an amount ranging from about 0.1 wt % to about 2 wt. When colloidal oatmeal is present, it is present in an amount ranging from about 0.007 wt % to about 5 wt %. When ascorbyl palmitate is present, it is present in an amount ranging from about 0.1 wt % to about 3 wt %.

In an embodiment, the viscosity of the composition ranges from about 50,000 cp to about 400,000 cp at 75° F. and 14.7 psig, and in another embodiment, from about 100,000 cp to about 300,00 cp at 75° F. and 14.7 psig, and in still another embodiment, from about 125,000 cp to about 225,000 at 75° F. and 14.7 psig, while in another embodiment, it ranges from about 150,000 cp to about 250,000 cp at 75° F. and 14.7 psig and in another embodiment, it ranges from about 175,000 cp to about 225,000 cp at 75° F. and in another embodiment at about 200,000 cp at 75° F. Pressure does not affect viscosity of gas-free compositions, but it does affect compositions containing gas. Low shear mixing is used to prevent accidental gas incorporation.

In an embodiment, the specific gravity of the topical composition ranges from about 0.83 to about 0.92 at 75° F. and 14.7 psig, while in another embodiment, it ranges from about 0.85 to about 0.91 at 75° F. and 14.7 psig, and in another embodiment, from about 0.87 to about 0.90 at 75° F. and 14.7 psig.

In an embodiment, the vegetable oil having an omega3 fatty acid content greater than 9 wt %, such as hemp oil and the marine oil, such as cod liver oil are present in a weight ratio of about 1:1 to about 2:1, respectively; and in another embodiment, in a weight ratio of about 1.1:1 to about 1.5:1 and, in still another embodiment, omega3 fatty acids are both present in almost equal amounts, i.e., about 1.03:1 to about 1.2:1 by weight, i.e., hemp oil to cod liver oil.

In an embodiment, marine oil, such as fish oil or algal oil, is present in the topical composition described herein, in an amount ranging from about 0.5 wt % to about 8 wt %, and, in another embodiment, from about 1 wt % to about 5 wt %, and in another embodiment, from about 2 wt % to about 3 wt %. Thus, the marine oil is either present in about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt % or about 8 wt %, or any value therebetween.

MCT is one of the ingredients of the present formulation. It is believed to be consumed (versus simple sugars) by cells for energy.

MCT drives macrophages to exhibit anti-inflammatory functions by enhancing mitochondrial respiration. In studies, MCT exhibited no effect on oxygen consumption in a non-stimulated M0-like status. Interestingly, however, under M1-like inflammatory conditions following LPS (lipopolysaccharide, an endotoxin) stimulation, MCT significantly increased OCR (oxygen consumption rate) for basal respiration, maximal respiration, and ATP production indicating that MCT not only suppresses inflammatory responses in M1 status, but also actively upregulates the switch of macrophage function towards M2-like anti-inflammatory repolarization. MCT enhances anti-inflammatory responses of macrophages via up-regulating mitochondrial respirations in accordance with previous reports, in which increased energy expenditure by medium-chain fatty acids (MCFAs) drives macrophages to exhibit anti-obesity and anti-inflammatory phenotypes.

Overall, up-regulated β-oxidation by MCT contributes to the anti-inflammatory M2-like status of macrophages, which may aid in the prevention and/or amelioration of inflammation.

MCT also indirectly reduces pain. Not by actively reducing pain like lidocaine, Novocain and similar “-caines,” but by not producing inflammatory lactate. For example, it was found that a composition comprising the combination of camphor, lidocaine and MCT, present in total in the formulation at 60 wt %, reduces pain deep in the muscular tissue. Camphor cools surface skin as it evaporates; lidocaine temporarily reduces pain in the dermis; high levels of MCT reduce deep pain by NOT producing inflammatory lactate. Together, the patient does not feel pain for hours.

Without wishing to be bound, it is believed that MCT can stimulate anti-inflammatory macrophages to decrease inflammation and to help create ROS (reactive oxygen species) that is produced during the healing stages of a wound or burn. However, excess ROS must be quenched. Without wishing to be bound, it is believed that free radical quenching is done by PUFA from vegetable and marine sources and antioxidants like squalene, Vitamin E and Vitamin C. It is well known that ROS is important to control pathogen growth, but excess ROS can cause tissue damage. Free radicals are initiated, then propagate (a chemical chain reaction) and then are terminated. The antioxidants neutralize (quench) excess ROS (terminate) so that the antimicrobial ROS benefits are retained without the tissue damage negatives.

In an embodiment, MCT is present in the composition of the disclosure in amounts ranging from about 40 wt % to about 75 wt %, and in another embodiment, it is present in an amount ranging from about 50 wt % to about 73 wt %, and in a further embodiment, it is present in an amount ranging from about 60 wt % to about 72 wt %. Thus, in an embodiment, MCT is present in either about 40 wt %, about 41 wt %, about 42 wt %, about 43 wt %, about 44 wt %, about 45 wt %, about 46 wt %, about 47 wt %, about 48 wt %, about 49 wt %, about 50 wt %, about 51 wt %, about 52 wt %, about 53 wt %, about 54 wt %, about 55 wt %, about 56 wt %, about 57 wt %, about 58 wt %, about 59 wt %, about 60 wt %, about 61 wt %, about 62 wt %, about 63 wt %, about 64 wt %, about 65 wt %, about 66 wt %, about 67 wt %, about 68 wt %, about 69 wt %, about 70 wt %, about 71 w %, about 72 wt %, about 73 wt %, about 74 wt % or about 75 wt % or any value therebetween.

The function of the monolaurin in the formulations described herein, is to help reduce the undesirable rancid smell of the marine oil, such as cod liver oil. Without wishing to be bound, it is believed that monolaurin also helps the marine oil be absorbed by unbroken skin. It is believed that the faster the marine oil is absorbed, the less oxidation odor is generated by exposure to air on the skin surface. However, without wishing to be bound, it is believed that it performs this function with the presence of the vegetable oil having an omega 3 content greater than 9 wt %, such as hemp oil and cetyl ester.

Monolaurin is a gel and is also acting as a gelling agent with respect to the other components present in the compositions of the gelled formulations described herein. To form a stable gel in these embodiments, the monolaurin is present in amounts of 6 wt % or greater.

Without wishing to be bound, it is believed that monolaurin, at concentrations of 6 wt % or greater, slow the rate of absorption significantly, but, in addition, is a permeation enhancer once the composition passes through the stratum corneum and the remainder of the epidermis. In other words, these gelled formulations are absorbed slowly through the stratum corneum and in the remainder of the epidermis. That is, the gels are slow-to-release, but once in the epidermis, monolaurin helps drag the marine oil, such as cod liver oil and the vegetable oil having omega 3 in amounts greater than 9 wt % thereof, such as hemp oil, into the dermis. Monolaurin, between 2% and 3.5 wt %, forms sticky gels. However, when the concentration of monolaurin is greater than or equal to 6 wt % of the composition, the sticky gel transforms into an elegant gel. Between 3.5 wt % and 6 wt %, the gel increases in strength but is unstable to even moderate shear. Once above or equal to 6 wt %, the gel is metastable, that is, it is stable in a thick form, but with minor mixing can become a thin gel. With intense mixing, the gel is broken. If broken gel is reheated to clarity, the process begins again. In other words, the thick gel is reproducible in that when all of the gel is melted, the gel reforms at ambient temperature.

In an embodiment, monolaurin is present in amounts ranging from about 7 wt % to about 14 wt % of the composition, while in another embodiment, it is present in amounts ranging from about 8 wt % to about 13 wt % and in another embodiment, it is present in an amount ranging from about 9 wt % to about 11 wt %. Thus, for example, monolaurin is present in either about 7 wt %, about 8 wt %, about 9 wt %, about 10 wt %, about 11 wt %, about 12 wt % about 13 wt % or about 14 wt % or any value therebetween.

In addition, another ingredient that helps remove the fishy odor in compositions described herein are the cetyl esters. In an embodiment, the cetyl esters are present in an amount ranging from about 4% to about 12% by weight. In another embodiment, the cetyl esters are present in an amount ranging from about 5% to about 8% by weight, and in another embodiment, about 5.5% to about 6.5% by weight. For example, cetyl esters may be present in either about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 10.5 wt %, about 11 wt %, about 11.5 wt %, about 12 wt % or any value therebetween. Further, it is believed that adding cetyl esters to the formulation creates a silky-smooth skin surface (finish) that is very pleasant to the touch.

With respect to the formulations described herein, without wishing to be bound, it is believed that the monolaurin helps drive the omega3 fatty acids through the stratum corneum, leaving only a small residual of potentially oxidizable fat on the skin surface. The Cetyl Esters, without wishing to be bound, provides a late-drying odor-occlusive layer over any potentially oxidizable fat. The net result is no odor after about 30 seconds.

A further means of reducing the rancid fish oil in compositions described herein is to use less of the marine oil, such as cod liver oil. Vegetable oil having an omega3 fatty acid content greater than 9 wt %, such as hemp oil, is another source of omega3 fatty acids. Marine oil, such as cod liver oil, provides very long chain omega3 fatty acids (C>18); vegetable oil having an omega3 fatty acid content greater than 9 wt %, such as hemp oil, provides long chain omega3 fatty acids (C=18). The body enzymatically takes omega3 fatty acids and either shortens or lengthens the chain length to make, for example, prostaglandins. If the omega3 fatty acid is C18 or less, the elongation process produces both inflammatory and anti-inflammatory compounds. If the omega3 fatty acid is C>18 (i.e., greater than or equal to C20), then the produced products are all anti-inflammatory. Wounds need some inflammatory compounds for example to control bleeding, as well as anti-inflammatory compounds, for example, to increase blood flow by reducing resistance to blood drainage.

In compositions of the present disclosure, it is advantageous to use vegetable oil having an omega3 fatty acid content greater than 9 wt %, such as hemp oil, in addition to marine oil, such as cod liver oil. This combination provides the most effective ratios of C18:C>18 omega3-fatty acids. In an embodiment, this ratio is about 1:1 (w/w). However, there is also the trade-off between inflammatory byproducts and anti-inflammatory products with odor control. Nevertheless, in compositions described herein, the combination of C18+C>18 omega3-fatty acids lower the foul fish odor smell. Thus, by using a greater amount of vegetable oil having an omega3 fatty acid content greater than 9 wt %, such as hemp oil, less marine oil, such as cod fish liver oil, is necessary. Accordingly, as described hereinabove, by utilizing more vegetable oil having an omega3 fatty acid content greater than 9 wt %, such as hemp oil, and reducing the amount of marine oil, such as cod liver oil, the present formulations reduce the rancid smell of the marine oil, such as cod liver oil. However, with the amounts of monolaurin and cetyl esters present herein, any rancid fish smell of the marine oil, such as cod liver oil, is considerably reduced or eliminated. As described herein, the addition of marine oil, such as hemp oil, cetyl esters and MCT and monolaurin, to the fish oil composition in the amounts described herein reduces or eliminates completely the rancid fish odor, and when the composition is applied to the skin of a patient, the burn or wound is reduced in size and the skin of the patient's skin is moisturized.

Fish oil odor is complex. Fish oil contains trimethylamine which is removed during deodorization (old fish smell). The PUFA fatty acids can be oxidized during processing, distribution or when exposed to air on the skin (“roadkill odor”). All sources of odor are controlled to get “credit” for eliminating fish odors.

In an embodiment, vegetable oil having an omega3 fatty acid content greater than 9 wt %, such as hemp oil, is present in an amount ranging from about 0.5 wt % to about 8 wt %, and, in another embodiment, from about 1 wt % to about 5 wt %, and in another embodiment, from about 2 wt % to about 4 wt %. Thus, the vegetable oil having an omega3 fatty acid content greater than 9 wt %, such as hemp oil, is either present in about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt % or about 8 wt %, or any value therebetween.

Another ingredient present in the present composition is rice bran wax (“RBW”). Rice bran wax is the vegetable wax which is extracted from the bran oil of rice (Oryza sativa). Rice Bran Wax is a high melting point vegetable wax. The main components of rice bran wax are aliphatic acids and alcohol esters. The aliphatic acids consist of C16 fatty acids such as palmitic acid, C22 fatty acid, such as behenic acid, C24 fatty acid, such as lignoceric acid; and higher wax acids The alcohol esters have higher carbon content than the aliphatic acids present therein. The alcohol esters of rice bran wax consist mainly of esters of C26 alcohols esters, such as cetyl alcohol and fatty acid ester of C30 alcohol esters, such as melissyl alcohol (C30). Rice bran wax is used as an emollient and exfoliant. Rice Bran Wax is used in cosmetics, skin care, and hair care. Formulators will find that it acts as a thickener, binder, plasticizer, and gelling agent. In sticks, it contributes to hardness, texture, strength, and mold release. Rice bran wax forms discrete crystals when it solidifies.

In an embodiment, rice bran wax is present in an amount ranging from about 2 wt % to about 12 wt % of the composition, and in another embodiment, from about 4 wt % to about 10 wt % and in another embodiment, from about 5 wt % to about 8 wt % of the composition Thus, in an embodiment, the rice bran wax is present in either about 2.0 wt %, about 2.5 wt %, about 3.0 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9.0 wt %, about 9.5 wt %, about 10.0 wt %, about 10.5 wt %, about 11.0 wt %, about 11.5 wt % or about 12.0 wt % or any value therebetween.

Additional components may be present in the present composition.

An optional ingredient present in the composition of the present disclosure is colloidal oatmeal. Colloidal oatmeal is made by grinding oat grain or Avena sativa into a fine powder. It is commercially available. It is an emollient—a substance that softens or soothes the skin—because it packs fats, proteins, vitamins, minerals, and other nutrients shown to benefit the skin. Colloidal oatmeal has been categorized by the FDA in 2003 as a skin protectant if the concentration is greater than 0.007 wt % (there is no regulatory upper limit). It also has anti-inflammatory and antioxidant properties that benefit skin. Antioxidants fight free radicals, which can damage a subject's body through oxidative stress if their numbers become too high. Colloidal oatmeal is also a process aid in that higher levels reduce oil leakage in the temperature abuse test.

In an embodiment, if present, colloidal oatmeal is present in amounts of less than 1 wt %, but greater than 0 wt %, such as for example, from about 0.007 wt % to about 5 wt %, and in another embodiment, from about 0.01 wt % to about 2 wt % and in another embodiment from about 0.01 wt % to about 0.5 wt %. Thus, when present, colloidal oatmeal is either present in the composition in about 0.007 wt %, about 0.008 wt %, about 0.009 wt %, about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.10 wt %, about 0.20% wt %, about 0.30 wt %, about 0.40 wt %, about 0.50 wt %, about 0.60 wt %, about 0.70 wt %, about 0.80 wt %, about 0.90 wt %, about 1.00 wt %, about 1.10 wt %, about 1.20% wt %, about 1.30 wt %, about 1.40 wt %, about 1.50 wt %, about 1.60 wt %, about 1.70 wt %, about 1.80 wt %, about 1.90 wt %, about 2.00 wt %, about 2.10 wt %, about 2.20% wt %, about 2.30 wt %, about 2.40 wt %, about 2.50 wt %, about 2.60 wt %, about 2.70 wt %, about 2.80 wt %, about 2.90 wt %, about 3.00 wt %, about 3.10 wt %, about 3.20% wt %, about 3.30 wt %, about 3.40 wt %, about 3.50 wt %, about 3.60 wt %, about 3.70 wt %, about 3.80 wt %, about 3.90 wt %, about 4.00 wt %, about 4.10 wt %, about 4.20% wt %, about 4.30 wt %, about 4.40 wt %, about 4.50 wt %, about 4.60 wt %, about 4.70 wt %, about 4.80 wt %, about 4.90 wt %, or about 5.00 wt %, or any value therebetween.

Another component that is optionally present in the present composition is ascorbyl palmitate. It is an ester formed from the reaction of ascorbic acid and palmitic acid, creating a fat-soluble form of vitamin C. Ascorbyl palmitate is a stable, lipid-soluble form of Vitamin C that is used in skincare products for its ability to protect the skin from free radicals. It also stimulates collagen production and reduces hyperpigmentation. Vitamin C is a naturally occurring antioxidant that is synthesized by most plants and animals from glucose.

In addition to its use as a source of Vitamin C, ascorbyl palmitate (AP) precipitates between the melting point of rice bran wax and monolaurin. It was determined experimentally that AP makes the mixture more stable. Rice bran wax first precipitates on cold metal surfaces as an amorphous wax, but after AP freezes, the amorphous wax dissolves and recrystallizes on the AP nuclei into a population of firm, discrete crystals. The rice bran wax-on-AP nuclei may also increase stability by stabilizing tripalmitin (C16:0 from palm oil) and vastly increase ambient viscosity. It is well known that rice bran wax can form crystals that orient in various configurations to bind oil (oleogel). The final orientation is not known currently, but the staged sequential freezing of rice bran wax, ascorbyl palmitate, monolaurin, cetyl esters and tripalmitin makes stable gels that withstand distribution temperature abuse and do not weep liquid fats.

In an embodiment, when ascorbyl palmitate is present, it is present in an amount ranging from about 0.1 wt % to about 3 wt %, and in another embodiment, from about 0.3 wt % to about 2 wt %, and in a further embodiment, from about 0.4 wt % to about 1 wt %. Thus, if present, it is present in the composition of the present disclosure in either about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2.0 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, about 3.0 wt %, or any value in-between.

A further optional ingredient present in the composition of the present disclosure is coconut oil. Coconut oil is added as a process aid. Moreover, it helps reduce any leakage during storage and transportation of product. In an embodiment, if present, it is present in about 0.1 wt % to about 2 wt %, and in another embodiment, from about 0.2 wt % to about 1 wt %, and in another embodiment, from about 0.3 wt % to about 0.5 wt %. Thus, if present, coconut oil is either present in about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt % or about 2 wt % or any value therebetween.

In an embodiment, the composition comprises the following ingredients: MCT, monolaurin, cetyl esters, marine oil, vegetable oils having an omega 3 fatty acid greater than 9 wt %, such as hemp oil, and coconut oil, rice bran wax, colloidal oatmeal and ascorbyl palmitate. Moreover, in another embodiment, the sum of the weight % (MCT+coconut oil)/sum of the weight percentage of (monolaurin+cetyl esters+rice bran wax) ranges from about 1 to about 5. In an embodiment, MCT is present in the composition in an amount ranging from about 40 wt % to about 75 wt %, monolaurin is present in an amount ranging from about 7 wt % to about 14 wt %; cetyl esters are present in an amount ranging from about 4 wt % to about 12 wt %; coconut oil is present in an amount ranging from about 0.1 wt % to about 2 wt %; marine oil is present in an amount ranging from about 0.5 wt % to about 8 wt %; vegetable oil having an omega 3 greater than 9 wt %, such as hemp oil, is present in an amount ranging from about 0.5 wt % to about 8 wt %; rice bran wax is present in an amount ranging from about 2 wt % to about 12 wt %; colloidal oatmeal is present in an amount ranging from about 0.007 wt % to about 5 wt %; and ascorbyl palmitate is present in an amount ranging from about 0.1 wt % to about 3 wt %, wherein the sum of the above-identified ingredients in the composition is no greater than 100 wt %., and wherein the sum of the weight percentages of all of the ingredients in the composition adds to 100 wt %. In an embodiment, the sum of the aforesaid ingredients is always 100 wt % of the composition, and in another embodiment, the sum of the weight percentages of the aforesaid ingredients minus MCT and colloidal oatmeal ranges from about 25 wt % to about 50 wt % of the composition. In an embodiment, the compositions described herein are collagen-free. In other words, MCT and colloidal oatmeal can be adjusted to make material balance “room” for alternate “cargo,” but the core of the composition does not change. In another embodiment, MCT is present in the composition in an amount ranging from about 50 wt % to 73 wt %, monolaurin is present in an amount ranging from about 8 wt % to about 13 wt %; cetyl esters are present in an amount ranging from about 5 wt % to about 8 wt %; coconut oil is present in an amount ranging from about 0.2 wt % to about 1 wt %; marine oil is present in an amount ranging from about 1.0 wt % to about 5.0 wt %; vegetable oil having an omega greater than 9 wt %, such as hemp oil, is present in an amount ranging from about 1 wt % to about 5 wt %; rice bran wax is present in an amount ranging from about 4 wt % to about 10 wt %; colloidal oatmeal is present in an amount ranging from about 0.01 wt % to about 2 wt %; and ascorbyl palmitate is present in an amount ranging from about 0.3 wt % to about 2 wt %, wherein the sum of the ingredients in the composition is no greater than 100 wt %. In another embodiment, MCT is present in the composition in an amount ranging from about 60 wt % to about 75 wt %, monolaurin is present in an amount ranging from about 9 wt % to about 11 wt %; cetyl esters are present in an amount ranging from about 5.5 wt % to about 6.5 wt %; coconut oil is present in an amount ranging from about 0.3 wt % to about 0.5 wt %, vegetable oil having an omega 3 greater than 9 wt %, such as hemp oil, is present in an amount ranging from about 2 wt % to about 3 wt %; marine oil is present in an amount ranging from about 2 wt % to about 3 wt %; rice bran wax is present in an amount ranging from about 6 wt % to about 8 wt %; colloidal oatmeal is present in an amount ranging from about 0.1 wt % to about 0.5 wt %; and ascorbyl palmitate is present in an amount ranging from about 0.4 wt % to about 1 wt %, wherein the sum of the ingredients in the composition is no greater than 100 wt %.

As an optional ingredient, FFA, such as coconut FFA, may be present. If present, it is present in less than 3 wt %, but greater than 0 wt % of the formulation. In an embodiment, it is present in amounts ranging from about 0.01 wt % to about 1.0 wt % and in another embodiment, from about 0.05 wt %. to about 0.8 wt % and in another embodiment, from about 0.1 wt % to about 0.6 wt %, and in another embodiment, from about 0.1 wt % to about 0.5 wt %. However, in some embodiments, it is present in about 0.1 wt % to about 0.4 wt %.

In another embodiment, caprylic acid may be present. Without wishing to be bound, it is believed that caprylic acid acts as a non-aqueous pH buffer.

If present, it is present in less than 3 wt %, but greater than 0 wt % of the formulation. In an embodiment, it is present in amounts ranging from about 0.01 wt % to about 1.0 wt % and in another embodiment, from about 0.05 wt %. To about 0.8 wt % and in another embodiment, from about 0.1 wt % to about 0.6 wt %, %, and in another embodiment, from about 0.1 wt % to about 0.5 wt %. However, in some embodiments, it is present in about 0.1 wt % to about 0.3 wt %.

Non-coconut vegetable oils, as defined herein, may additionally be present in any of the formulations described. As defined for purposes of this disclosure, the term “vegetable oil having an omega3 fatty acid content greater than 9 wt %” is different from the term “non-coconut vegetable oil”, as described hereinabove, and coconut oil. The “vegetable oil having an omega3 fatty acid content greater than 9 wt %” and “non-coconut vegetable oil” do not contain any coconut oils. These non-coconut vegetable oils include palm oil (including, but not limited to red palm oil and RBD palm oil, and red palm concentrate). The non-coconut vegetable oil, if present, may be present in an amount ranging from about 0.1 wt % to about 5.0 wt % and in another embodiment, from about 2.0 wt % to about 4.0 wt %. In an embodiment, coconut oil is present in combination with palm oil. This combination reduces the amount of oleic fatty acid (from the palm oil) and replaces it with lauric acid (C12:0) and myristic acid (C14:0) both from the coconut oil. Although palm oil, with its high concentration of Vitamin E, has skin moisturizing characteristics, what has been found is that the presence of coconut oil and red palm disrupts pathogen growth in wound healing products.

In another embodiment, squalene/Vitamin E is added in amounts ranging from about 0.05 wt % to about 1.0 wt % and in another embodiment from about 0.1 wt % to about 0.3 wt %. Squalene/Vitamin E is an extract from red palm oil and is commercially available, such as from PhytoGaia Corp. in Malaysia. The squalene/Vitamin E extract is the natural ratio defined by the red palm fruit and is present in the commercial product in a range of about 0.01 wt % to about 1 wt %, for example, at about 0.2 wt %. Squalene/Vitamin E addition increased the tactile acceptability of the composition of the present disclosure and does not stain the skin.

Without wishing to be bound, it is thought that the addition of different antioxidants, for example, Vitamin E and Ascorbyl Palmitate, increase the antioxidant capacity of the composition because each has its own mechanism of action. Thus, replacing Vitamin E-rich palm oil with nil Vitamin E coconut oil which does not contain significant concentration of Vitamin E and adding squalene/Vitamin E increases the in vitro antioxidant capacity (in combination with oxygen barrier packaging) but decreases the in vivo antioxidant capacity and increases the pro-oxidation ROS sufficiently that infection is controlled without a negative effect on the healing rate or skin staining. Adding MCT to the coconut oil and Vitamin E accelerates the healing process by concentrating the ROS on the surface where there can be infection. It is counterintuitive to use the same composition to be an antioxidant during storage and distribution and a source of pro-oxidant, antimicrobial ROS when the composition is applied to the wound, as in the present formulation.

In another embodiment, the non-coconut vegetable oil is RBD palm oil, without the presence of palm kernel oil. In this embodiment, if present, the RBD palm oil is present ranging from about 1% wt % to about 5% wt %, and in another embodiment, from about 2 wt % to about 4 wt %, and in another embodiment, from about 2.5 wt % to about 3.5 wt %. RBD palm oil is indicated for use on intact skin. The Vitamin E in the palm oil is advantageously used on intact skin; any red/orange residual color on the skin from the application of red palm concentrate on the skin is perceived by consumers as a defect. RBD palm oil does not discolor the skin. The previously described rapid absorption with an odor-occlusive silky-smooth film makes this a consumer-acceptable composition to bring topical omega3 fatty acids from the marine oil and vegetable oil having omega3 fatty acid content of more than 9.0 wt %, such as hemp oil, to intact skin.

In an embodiment, water soluble zinc salts may be present in the composition. Examples of zinc salts include zinc acetate, zinc fluoride, zinc chloride, zinc chlorate, zinc perchlorate, zinc nitrate, zinc sulfate, and the like. When present, the zinc salts are present in a range of about 0.02 wt % to about 0.15 wt %, and in another embodiment, from about 0.04 wt % to about 0.12 wt % and in another embodiment, from about 0.06 wt % to about 0.1 wt %. Thus, for example, zinc salt may be present in about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.11 wt %, about 0.12 wt %. about 0.13 wt %, about 0.14 wt %, about 0.15 wt %, and any value therebetween. Without wishing to be bound, it is believed that Zinc ions are important co-factors along with matrix metalloproteases (MMP). MMP are the enzymes that modify weak scar collagen during wound repair.

Moisturizers may additionally be present. For example, lauric triglyceride and/or oleic triglycerides are moisturizers that may be added to any of the formulations described herein. However, lauric triglyceride is a better skin moisturizer than oleic triglyceride.

Additionally, the composition can comprise, in addition to the components described hereinabove, any one or more of therapeutically active compounds and pharmaceutically and/or cosmetically acceptable diluents, excipients or carriers. For example, the composition of the present disclosure can be used as a carrier for drugs useful for treating skin conditions or burns, as long as the drug is oil soluble or lipophilic. The term “lipophilic” as used in the present disclosure implies that the drug is entirely lipophilic or has both a hydrophilic and lipophilic character, but more lipophilic character than hydrophilic character. The term lipophilic therefore encompasses solubilities which range from exclusive solubility in non-polar, water-immiscible organic solvents to complexes having solubility both in these solvents and non-aqueous water immiscible solvents. The gradation of lipophilicity of the compositions of the present disclosure can be established by reference to partition coefficients using n-octanol/water, or n-octanol/buffer, or n-octanol/saline. In general, those drugs having n-octanol/saline partition coefficients greater than about 10 and especially 100 are useful drugs for which the present compositions can be carriers. Examples of drugs include anesthetics, such as lidocaine (excluding water soluble lidocaine salts, such as lidocaine hydrochloride), in pain killing effective amounts. For example, the compositions of the present disclosure may contain a safe and effective amount of a topical anesthetic and/or analgesic which numbs tissue and reduces pain associated with the skin condition or wound. Examples of topical anesthetic drugs include benzocaine, lidocaine, bupivacaine, chlorprocaine, dibucaine, etidocaine, mepivacaine, tetracaine, dyclonine, hexylcaine, procaine, benzethonium chloride, methylbenzethonium chloride and benzalkonium chloride, cocaine, ketamine, pramoxine, phenol, camphor, oil of camphor and pharmaceutically acceptable salts thereof that are lipophilic, e.g., have a n-octanol/saline partition coefficient greater than about 10. When present, the drug is present in a safe and effective amount. For example, in an embodiment, it may be present in an amount ranging from about 0.5% to about 4.0 wt %. However, the API (Active Pharmaceutical Agent) is lipophilic. Other non-inclusive examples include lipophilic salts such as Amphotericin B and itraconazole. Further, the present composition may be a carrier for statins. Statins are a family of useful active pharmaceutical ingredients. Statins are well known as topical wound healing compounds. Valsartan is an example of a statin; its molecular weight is less than 500 daltons. Other statins have a molecular weight greater than 500 daltons may be present in the composition described herein as an active pharmaceutical ingredient.

Further, other drugs may be present. For example, the drug may be an element of the periodic table, such as bismuth or it may be an inorganic salt, such as zinc oxide. These elements are not lipophilic but can be entrapped by the cooled structure of the carrier.

Besides the components listed above, the compositions of the present disclosure may contain other additional optional ingredients. For example, the composition may contain fragrances, including water soluble non-alcohol containing fragrances or perfumes, such as to further conceal any remaining rancid fishy smell, such as Fragrance B. If present, the perfumes, or fragrances, such as lemon oil, lavender and vanilla and the like, are present in less than 2 wt %, such as for example from about 0.01 wt % to about 1.5 wt %. Oil soluble fragrances include such components as vanilla, lemon oil, lavender, and the like.

In addition, the composition may comprise thickening agents (e.g., wax, beeswax, PEG 4000, PEG 600, hard paraffin) and/or gelling agents (e.g., hydroxypropyl cellulose). In general, thickening agents are not required when cooling is slow enough that each precipitation is complete before the onset of the next precipitation. The composition may also comprise emulsifying agents, (e.g., wool wax alcohol), stabilizing agents (e.g., polyoxyethylene sorbitan monolaurate, carboxy methyl cellulose), dispersing agents (e.g., sodium oleate, propylene glycol), suspending agents (e.g., methyl cellulose, chitosan, accacia, carboxymethyl cellulose, tragacanth, pectin), and/or coloring agents (e.g., dyes, lackes). In addition, buffers to control pH, such as disodium hydrogen phosphate/sodium dihydrogen phosphate, agents to adjust osmolarity such as sodium chloride, and stabilizers such as EDTA., may be present. Further, oil soluble anti-oxidizers, such as Vitamin E, and astaxanthin can be added. Oil soluble vitamins, such as Vitamin D, can be present, inherently with the cod liver oil or added as a supplement. In addition, oil-soluble coloring agents may be added. In addition, sebum and squalane can be added. Further, benzethonium chloride, methylbenzethonium chloride and benzalkonium chloride may be present.

Other additives may be present in the composition described herein. For example, antioxidants and preservatives may be added. Examples of antioxidants include, but are not limited to, tocopherols, ascorbic acid, Vitamin K, sodium pyrosulfite, butylhydroxytoluene, butylated hydroxyanisole, edetic acid, and edetate salts, and mixtures thereof. Examples of preservatives include, but are not limited to, citric acid, tartaric acid, lactic acid, malic acid, acetic acid, benzyl alcohol, benzoic acid, and sorbic acid.

A wide variety of optional components/ingredients may be included in the compositions of the present disclosure. For example, the compositions may include absorbents, abrasives, anticaking agents, antifoaming agents, antimicrobial agents, binders, biological additives, chemical additives, biocides, denaturants, cosmetic astringents, drug astringents, external analgesics, film formers, humectants, opacifying agents, pigments, essential oils, skin sensates, emollients, skin soothing agents, pH adjusters, plasticizers, propellants, reducing agents, additional skin-conditioning agents, skin penetration enhancing agents, skin protectants, solvents, suspending agents, solubilizing agents, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins and derivatives thereof, and natural extracts. Such other materials are known in the art. Nonexclusive examples of such materials are described in Harry's Cosmedocology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. ad., deNavarre (Van Nostrand 1962-1965); and in The Handbook of Cosmetic Science and Technology, 1st Ed. Knowlton & Pearce (Elsevier 1993). These additional ingredients are jointly referred to as “cargo.” As used herein, the term “cargo” the term “cargo” are the ingredients present in the composition that are solids denser than the liquid oils (e.g., >0.92 specific gravity) at the onset temperature of Exotherm #1, defined hereinbelow. For example, cargo does not include MCT, monolaurin, cetyl esters, marine oil, vegetable oils having an omega 3 fatty acid content greater than 9 wt %, rice bran wax, and ascorbyl palmitate. The cargo is present in a range from about 0.01 wt % to about 5 wt %.

Rice bran wax crystals are not cargo because the specific gravity of RBW crystals<0.92 at the onset of Exotherm #1. Colloidal oatmeal is cargo because its specific gravity>0.92. Cetyl esters are not cargo because cetyl esters are a liquid melt at the onset temperature of Exotherm #1. Zinc acetate @≤0.08% are not cargo because zinc acetate is soluble in oil up to 0.08%. At concentrations>0.08% higher, zinc acetate is cargo because the undissolved salt has a specific gravity>0.92. Bismuth is cargo because its specific gravity>0.92. Ascorbyl palmitate is not cargo because it serves as the nuclei for RBW needle crystal formation that as co-crystals have a specific gravity<0.92. Pharmaceutical API are cargo because their specific gravity is >0.92 at the onset of Exotherm #1.

It was found experimentally that cargo can be added between the freezing point of AP and the freezing point of monolaurin. If added after the freezing point of monolaurin, the product is not stable. If added before the freezing point of AP, it is not stable. In some formulations, colloidal oatmeal had to be increased (e.g., to 1.8%) to assure that the incompatible cargo did not destabilize the mixture.

In an embodiment, MCT is present in the composition in the greatest amount. In an embodiment, MCT is present in an amount ranging from about 40 wt % to about 75 wt %, monolaurin is present in an amount ranging from about 7 wt % to about 14 wt %; cetyl esters are present in an amount ranging from about 4 wt % to about 12 wt %; fish oil is present in an amount ranging from about 0.5 wt % to about 8 wt %; vegetable oil having an omega 3 fatty acid content greater than 9 wt %, such as hemp oil, is present in an amount ranging from about 0.5 wt % to about 8 wt %; rice bran wax is present in an amount ranging from about 2 wt % to about 12 wt %; wherein the sum of the above-identified ingredients in the composition is no greater than 100 wt %. Optionally, if present, coconut oil is present in an amount ranging from about 0.1 wt % to about 2 wt %; colloidal oatmeal is present in an amount ranging from about 0.007 wt % to about 5 wt %; and ascorbyl palmitate is present in an amount ranging from about 0.1 wt % to about 3 wt %. In another embodiment, MCT is present in the composition in an amount ranging from about 50 wt % to 75 wt %, monolaurin is present in an amount ranging from about 8 wt % to about 13 wt %; cetyl esters are present in an amount ranging from about 5 wt % to about 8 wt %; marine oil is present in an amount ranging from about 1.0 wt % to about 5.0 wt %; vegetable oil having an omega 3 fatty acid content greater than 9 wt %, such as hemp oil, is present in an amount ranging from about 1 wt % to about 5 wt %; and rice bran wax is present in an amount ranging from about 4 wt % to about 10 wt %; wherein the sum of the above-identified ingredients in the composition is no greater than 100 wt %. Optionally, when present, coconut oil is present in an amount ranging from about 0.2 wt % to about 1 wt %; colloidal oatmeal is present in an amount ranging from about 0.01 wt % to about 2 wt %; and ascorbyl palmitate is present in an amount ranging from about 0.3 wt % to about 2 wt %. In another embodiment, MCT is present in the composition in an amount ranging from about 60 wt % to about 75 wt %, monolaurin is present in an amount ranging from about 9 wt % to about 11 wt %; cetyl esters are present in an amount ranging from about 5.5 wt % to about 6.5 wt %; vegetable oil having an omega 3 fatty acid content greater than 9 wt %, such as hemp oil, is present in an amount ranging from about 2 wt % to about 4 wt %; marine oil is present in an amount ranging from about 2 wt % to about 4 wt %; rice bran wax is present in an amount ranging from about 5 wt % to about 8 wt %, wherein the sum of the ingredients in the composition is no greater than 100 wt %. If present, coconut oil is present in an amount ranging from about 0.25 wt % to about 0.5 wt %, colloidal oatmeal is present in an amount ranging from about 0.015 wt % to about 0.05 wt %; and ascorbyl palmitate is present in an amount ranging from about 0.4 wt % to about 1 wt %.

All of the possible combinations and permutations of the components listed herein are contemplated to be within the scope of the present compositions. However, the sum of all of the components present in the compositions herein adds to 100 wt %. In an embodiment, sum of wt % of marine oil; vegetable oil having an omega-3 fatty acid content greater than 9 wt %, such as hemp oil; monolaurin; medium chain triglycerides (“MCT”); cetyl esters; rice bran wax; coconut oil, colloidal oatmeal and ascorbyl palmitate (when coconut oil, ascorbyl palmitate and colloidal oatmeal are present) are present in the compositions of the present disclosure in an amount ranging from about 54.2 wt % to about 100 wt % and in another embodiment, from about 60 wt % to 100 wt %, and in another embodiment, from about 75 wt % to 100 wt %, and in another embodiment from about 80 wt % to 100 wt %, and in a further embodiment, from about 90 wt % to 100 wt % and in another embodiment, from about 95 wt % to 100 wt % and in further embodiments from about 96 wt % to 100 wt %, or about 97 wt % to 100 wt %, about 98 wt % to 100 wt %, or about 99 wt % to 100 wt %, and any number therebetween, such as about 55 wt %, about 56 wt %, about 57 wt %, about 58 wt %, about 59 wt %, about 60 wt %, about 61 wt %, about 62 wt %, about 63 wt %, about 64 wt %, about 65 wt %, about 66 wt %, about 67 wt %, about 68 wt %, about 69 wt %, about 70 wt %, about 71 wt %, about 72 wt %, about 73 wt %, about 74 wt %, about 75 wt %, about 76 wt %, about 77 wt %, about 78 wt %, about 79 wt %, about 80 wt %, about 81 wt %, about 82 wt %, about 83 wt %, about 84 wt %, about 85 wt %, about 86 wt %, about 87 wt %, about 88 wt %, about 89 wt %, about 90 wt %, about 91 wt %, about 92 wt %, about 93 wt %, about 94 wt %, about 95 wt %, about 96 wt %, about 97 wt %, about 98 wt %, about 99 wt %, or about 100 wt %, or any value therebetween. The sum wt % of marine oil; vegetable oil having an omega-3 fatty acid content greater than 9 wt %, such as hemp oil; monolaurin; medium chain triglycerides (“MCT”); cetyl esters; coconut oil; rice bran wax; colloidal oatmeal and ascorbyl palmitate cannot be greater than 100 wt %. Moreover, the sum of all of the ingredients in the composition of the present disclosure equals 100 wt %.

In an embodiment, MCT is present in an amount that ranges from about 10 to about 18 times the sum of the marine oil and vegetable oil having an omega-3 fatty acid content greater than 9 wt %, and in another embodiment, from about 12 times to about 15 times the sum of the marine oil and vegetable oil having an omega-3 fatty acid content greater than 9 wt %. In another embodiment, MCT is present in an amount from about 5 to about 16 times the sum of the marine oil, and vegetable oil having an omega-3 fatty acid content greater than 9 wt %, and coconut oil, and in another embodiment, from about 7 to about 14 and in another embodiment, from about 9 to about 12 times the sum of vegetable oil having an omega-3 fatty acid content greater than 9 wt %, marine oil and coconut oil.

In another embodiment, the weight ratio of monolaurin to cetyl esters ranges from about 1.2 to about 3.0, and in another embodiment, from about 1.4 to about 2.5, and in another embodiment, from about 1.5 to about 2, and in a further embodiment, about 1.7.

Also determined experimentally is that the sum of the weight percentage of monolaurin, coconut oil (when present), cetyl ester, palm oil, squalene/Vitamin E, and rice bran wax ranges from about 30 wt % to about 35 wt % and in another embodiment, about 32% of the formulation. In another embodiment, the weight percentage of MCT+colloidal oatmeal+additional “cargo” sum to about 65 wt % to about 75 wt % and in another embodiment, about 68 wt %. This narrow range balanced an aesthetically robust topical cream with delivery of often incompatible cargo. When rubbed in, cargo is covered by a wax/fat semi-occlusive film and then a surface active, layered, silky-smooth wax ester layer. The net result is reduced TEWL (better hydration; prevention of malodor creation and then release), intimate contact of cargo and skin and rapid absorption of lipophilic compounds.

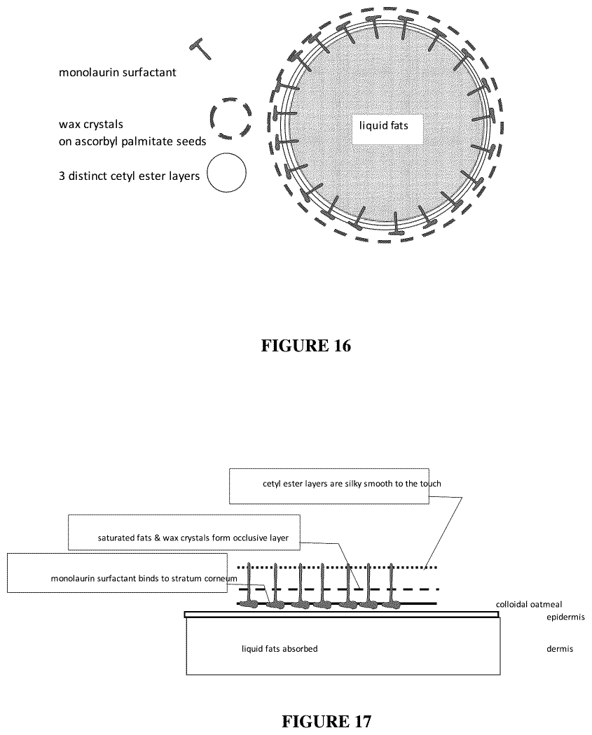

In an embodiment, the penetration ratio is greater than 1. As used herein, the term “penetration ratio” refers to the sum by weight of the triglycerides of C8 and C10 saturated fatty acids (as triglycerides)/sum by weight of the other triglycerides, including saturated and unsaturated fatty acids larger than C10 present in a formulation of the present disclosure. In an embodiment, the penetration ratio ranges from 1 to about 11, and in another embodiment, from about 5 to about 10, and in a further embodiment, from about 7 to about 10, and in an even further embodiment, from about 8 to about 10. Fatty acids are mainly composed of long chains of hydrocarbons ending in a carboxyl group. By definition, a fatty acid is a molecule of the formula R₄COOH, wherein R₄ is an organic group, such as a hydrocarbyl group, attached to the carboxyl group, COOH, containing an even number of carbon atoms and at least 6 carbon atoms and up to 30 carbon atoms. The carbon atoms may be a straight chain or branched or aromatic or a combination of aromatic and straight or branched chains. A fatty acid is saturated when the bonds between carbon atoms are all single bonds and is unsaturated when one or more of the bonds between the carbon atoms are unsaturated, e.g., carbon-carbon double or triple bonds or when the fatty acid contain an aromatic functionality. A triglyceride is, by definition, 3 fatty acids esterified to the three hydroxyl groups of a glycerol molecule (triacylglycerol). The fatty acids esterified to the glycerol molecule may be the same or different. Since the components used herein are natural products and/or are commercially available, the wt % of triglycerides of saturated fatty acids and triglycerides of unsaturated fatty acids in each composition can be easily determined by adding up the percentage by weight of the triglycerides of the saturated fatty acids and the unsaturated fatty acids in the various components present in each of the formulations. As a shorthand notation, it is referred to as (C8+10)/(sum other triglycerides). As described herein, the core of the composition (e.g., oils, waxes, MCT) form a structure, such as depicted in FIG. 16. Within that structure other lipophilic and lipophobic compounds, i.e., cargo, can be impounded. The basic structure is stable and surprisingly, destabilizing compounds can be entrapped inside of the monolaurin spheres without destabilizing each sphere. For example, high specific gravity compounds like bismuth, salts like zinc oxide, oily materials like squalene and Vitamin E have been captured in the monolaurin spheres.

In an embodiment, the sum of omega 3 fatty acid content is present in an amount ranging from 0.5 wt % to about 8 wt %, and in another embodiment, from about 0.9 wt % to about 6 wt % and in another embodiment from about 1 wt % to about 2 wt %.

In an embodiment, the PUFA ratio ranges from about 0.5 to about 2.0, and in another embodiment, the ratio ranges from about 0.8 to about 1.5, and in a further embodiment, the ratio is about 1:1.

In the composition of the present disclosure, when present, ascorbyl palmitate forms nuclei for rice bran wax nanocrystal formation; and the ascorbyl palmitate/rice bran wax subsequently becomes a nucleus for saturated fat crystals (often described as “stacked chairs”). In preparing the compositions described herein, colloidal oatmeal addition, if present, is withheld until after the ascorbyl palmitate (AP) and rice bran wax (RBW) crystallization is complete, and crystal integrity of the RBW crystals and separately crystal integrity of the saturated fat crystals is improved. The mixture is phase stable during storage and distribution. TEWL (Trans Epidermal Water Loss), which is the amount of water that passively evaporates through skin to the external environment due to water vapor pressure gradient, is reduced.

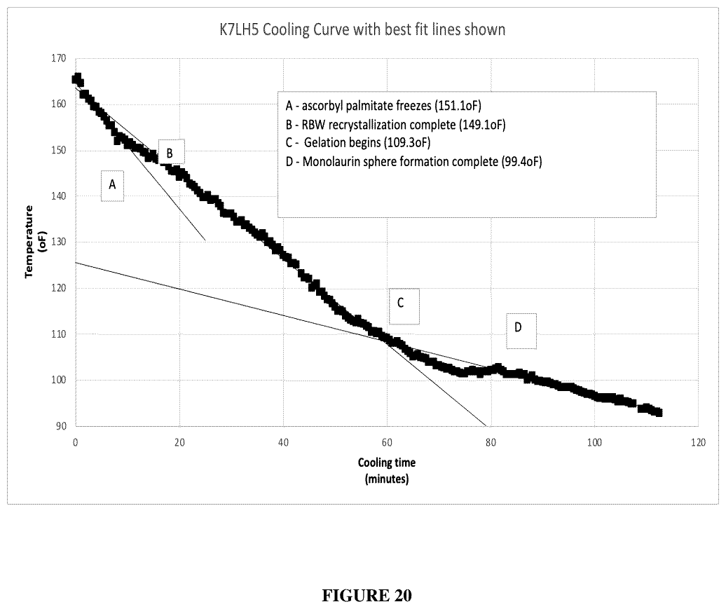

The composition of the present disclosure is prepared by placing all of the ingredients of the composition, except colloidal oatmeal and soluble zinc ions and other cargo in a vessel and heating the contents to sufficient temperature to melt all of the fat, waxes, and fatty acids that are present in the composition. For example, the contents of the vessel are heated together until the mixture of MCT oil, monolaurin, vegetable oil having an omega3 fatty acid content greater than 9 wt %, marine oil, cetyl esters, rice bran wax, and ascorbyl palmitate, if present melt, and any other fat, wax or fatty acid or any other non-cargo ingredient, i.e., any ingredient that that may be present, such as squalene/Vitamin E and/or FFA present is clear. In an embodiment, the vessel is heated to temperatures ranging from about 180° F. to about 185° F. for sufficient time to melt the fatty acids, the waxes, and the fat. It was experimentally determined that some normally insoluble compounds can be solubilized at low levels in the hot oil mixtures. For example, 0.7% lidocaine is soluble; zinc acetate is soluble @0.08%, benzethonium chloride is soluble @0.11%. The mixture is then mixed in a low shear mixer at a low shear rate, while allowing the temperature of the components of the mixture to cool slowly (<1° C./min) until the temperature of the melt ranges from about 130° F. to about 120° F. The low shear rate is such that the specific gravity of the composition does not change upon mixing. The rate is sufficiently low so that gas is not absorbed in the composition during mixing. In addition, air is not allowed to be in the composition. For example, in a standard beater agitator in a table-top mixer, the speed of the mixer is set at the second lowest setting, which is 1 (“Stir” is the lowest setting.) In an embodiment, all the oils and waxes and lipophilic compounds are heated until the mixture is clear. Then ambient cargo is added to drop the temperature of the combined mix, ensuring that the combined temperature is greater than 120° F. Cargo addition can take place at any time during the heating cycle, or cargo can be added after AP crystallization (Point A in FIG. 20). In an embodiment, when cargo is added during cooling, the cargo is fully wetted before the sensible heat of the cold cargo lowers the combined temperature below Point C (FIG. 20). The endpoint for cargo addition occurs once the previously falling temperature begins to increase, i.e., in mixing, the cargo is physically suspended via gentle mixing while the C-to-D (FIG. 20) exotherm is going to completion. Too much mixing destroys the sphere formation; too little mixing allows cargo to drop out and not be entrained. In the standard mixing paddle, a setting of “1” is used. In the present process, the cargo is kept physically suspended via gentle mixing while the C-to-D (FIG. 20) exotherm is going to completion.

The endpoint for cargo addition occurs once the previously falling temperature begins to increase (mixture visibly begins to gel). This first exotherm occurs when monolaurin begins to form an encapsulating structure. If cargo addition occurs after encapsulation, the cargo is not encapsulated and will escape during storage and distribution. In one embodiment, coconut oil was added before and after this exotherm. When 0.4% coconut oil was added before the exotherm, the skin feel was silky smooth; when added after the exotherm, the skin feel was unacceptably greasy. Coconut oil is an optional ingredient, typically included at less than 5 wt % because coconut oil can plug pores (comedogenic). In other words, the plus cosmetic elegance of coconut oil can be offset by the negative of plugged pores. In an embodiment, the mixing is performed instead at 100 rpm in a mixing bowl, such as a Kitchen Aid model #KSM7586 mixer with a dough hook rotating attachment, during rice bran wax crystallization (to mix without splashing), but the rotating attachment is changed to a standard paddle mixer (to increase shear just before monolaurin freezing) and before cargo addition. In an embodiment, the mixing bowl is maximally filled with mixture (just below the bottom of the top structure of the rotating attachment). This speed and mixing attachment combination is the lowest shear setting attainable with this mixer (dough hook) and a modestly higher shear with the paddle mixer (mixing paddle). [A whisk attachment is not used as it incorporates gas.] Rice bran wax crystals are bigger with the lower shear dough hook (100 rpm). Nano-liquid-capsule formation is abrupt and complete with a little more shear (mixing paddle, 100 rpm).

The energy balance of the mixing plan includes heating the mixture to a preset temperature (clear liquid), cooling abruptly by adding ambient cargo, adding “work” via the mixing energy, and cooling the mixture either by cooling in a heat/cool kettle or allowing the agitated bowl to lose heat naturally. Heat removal is the sum of mixing work, exothermic phase change added minus transfer of heat through the bowl wall into the atmosphere.

If any residue is present on the walls of the mixer as a result of the mixing, the residue is scraped off the walls and placed into the liquid mixture. Colloidal Oatmeal, if present, is then added to the mixture, and it is stirred under low shear in a low shear mixer until the temperature of the mixture ranges from about 95° F. to about 85° F. The sheer rate is less than the rate of mixing that would allow colloidal oatmeal to settle. The shear rate is dependent on the cargo. When colloidal oatmeal is present, the colloidal oatmeal cannot be allowed to settle, nor can gas be incorporated into the mixture. With lipophilic cargo, less agitation is optimal. With high density cargo, more mixing is required. Thus, the amount of mixing is cargo dependent.

At this end point (in an embodiment, about 90° F.), the mixture is smoother and lighter (i.e., reflects more light). The mixing is stopped, and the mixture is allowed to cool to ambient temperature in the mixing bowl. Between 800 and 75° F., saturated fats (C12:0 to C14:0) precipitate, materially increasing viscosity. The contents of the mixture are transferred to a low shear, viscous drum emptying system, such as a Viscotec (Viscotec America, Atlanta GA). The viscosity of the ambient mixture in its mixing bowl is >400,000 cp. Simply transferring the ambient mixture to a storage drum, with minimal shear, reduces the viscosity to about 150,000 to about 250,000 cp. Thereafter the viscosity is constant, at about 150,000 to about 250,000 cp. The composition is then packaged with as close to zero shear as is possible (post cooling shear destabilizes the composition). Since the composition is viscous, it is fed into the packaging through an extruder-style metering pump. In addition, the packaging, in an embodiment, dispenses the composition through an extruder dispensing pump directly into the ultimate consumer package.

A composition that comprises the stabilized formulation can be formulated for topical application as described herein and sealed in an air-tight container suitable for a single use. Such a composition can be used to treat and minimize scarring (by increasing skin hydration) in any of the skin conditions, burns and/or wounds described herein.

As used herein, a wound is defined as an injury to living tissue of the mammal in which the skin is cut or broken. It includes incisions, cuts, including paper cuts and shaving cuts. However, as defined herein, the wound may or may not be accompanied by bleeding. Examples of wounds treatable by the composition described herein include, but are not limited to, incisions (including surgical incisions), lacerations, abrasions (such as in dermabrasion and microderm abrasions), ulcers, and the like. In some embodiments, the wound is a diabetic wound ulcer.

“Skin disorders,” as used herein, refer to chapped skin and other skin conditions where the skin is broken. Examples of skin disorders include acne, psoriasis, eczema, dermatitis, alopecia, rosacea, burns, chapped skin, poison ivy, shingles, and the like.

The term “acne” is meant to include any skin condition where a skin pore becomes blocked and/or thereby becomes inflamed. The term acne includes without limitation superficial acne, including comedones, inflamed papules, superficial cysts, and pustules; and deep acne, including deep inflamed modules and pus-filled cysts. Specific acne conditions can include, but are not limited to, acne vulgaris, acne comedo, papular acne, premenstrual acne, preadolescent acne, acne venenata, acne cosmetica, pomade acne, acne detergicans, acne excoriee, gram negative acne, acne rosacea, pseudofolliculitis barbae, folliculitis, perioral dermatitis, and hiddradenitis suppurativa. Acne is a common inflammatory pilosebaceous disease characterized by comedones, papules, pustules, inflamed nodules, superficial pus-filled cysts, and (in extreme cases) canalizing and deep, inflamed, sometimes purulent sacs. Acne involves an interaction between hormones, keratinization, sebum, and bacteria that somehow determines the course and severity of acne. It often begins at puberty, when the increase in androgens causes an increase in the size and activity of the pilosebaceous glands. The earliest microscopic change is thought to be intrafollicular hyperkeratosis, which leads to blockage of the pilosebaceous follicle with consequent formation of the comedo, composed of sebum, keratin, and microorganisms, particularly Propionibacterium acnes. Lipases from P. acnes break down triglycerides in the sebum to form free fatty acids (FFA), which irritate the follicular wall. Retention of sebaceous secretions and dilation of the follicle may lead to cyst formation.

Skin conditions also include, but are not limited to, dermatological conditions linked to disorders of keratinization involving differentiation and proliferation, in particular, acne vulgaris, comedonic or polymorphic acne, nodulocystic acne, acne conglobata, senile acne and secondary acnes such as solar, drug or occupational acne; for other types of keratinization disorders especially ichthyoses, ichthyosiform conditions, Darier's disease, palmoplantar keratoderma, leukoplakia and luecoplakiform conditions or lichen and lichen planus; dermatological disorders having an inflammatory or immunoallergic component, in particular, all forms of psoriasis, either cutaneous, mucosal or ungual, and psoriatic rheumatism, and cutaneous atopy such as eczema or respiratory atopy, dry skin, inflammation of the skin, solar erythema, skin allergies or other skin disorders of the epidermis and dermis.

Psoriasis is a skin condition characterized by hyperplasia of keratinocytes resulting in thickening of the epidermis and the presence of red scaly plaques. The lesions in this chronic disease typically are subject to remissions and exacerbations. There are several patterns, of which plaque psoriasis is the most common. Guttate psoriasis, with raindrop shaped lesions scattered on the trunk and limbs, is the most frequent form in children, while pustular psoriasis is usually localized to the palms and soles. The classical inflammatory lesions vary from discrete erythematous papules and plaques covered with silvery scales, to scaly itching patches that bleed when the scales are removed. Psoriasis is a condition in which cell proliferation is increased up to 10 times the normal rate for an individual. The skin is the largest portion of the human body which is comprised of cells within three skin layers. Each of the skin layers is in a constant state of growth with the outer layer being formed of predominantly dead tissue which is naturally being discarded at a normal rate. Replacement of cells from underlying layers is accomplished by cell division and maturation where cells move upwardly and outwardly at a rate which varies dependent upon the age, sex, and/or health of an individual. Psoriasis causes an increased turnover of cells, which in turn increases the rate of cell growth and cell death. This increased rate of cell growth and cell death may result in injuries and/or disorders which accompany the increased synthesis of all tissue components and further elevate the strain placed upon skin or other tissue and the bio-synthetic capabilities of the cells within the affected area.

The terms eczema and dermatitis are skin conditions, as used herein, generally involve severe inflammation of the skin, usually with redness, swelling, oozing, rusting, or scaling of lesions which are usually itchy. Eczema may take the form of contact dermatitis (due to skin contact with the cause) or atopic dermatitis in individuals who are “atopic” or allergic by nature. If the scalp is involved, the disorder is known as seborrheic dermatitis. Dermatitis can be caused by chemicals, plants, shoes, clothing, metal compounds and even medicines used to treat dermatitis. In atopic dermatitis environmental temperature, humidity changes, bacterial skin infections, airborne allergens, and garments, e.g., wool, may all bring about dermatitis. In an embodiment, the collagen paste described herein is used as a skin scrub. The ground sea salt and fish collagen gently abrade damaged and dead cells from the skin surface. Simultaneously, the oils are absorbed. The combination of mechanical removal of damaged cells and concurrent feeding of viable cells gives the skin a vibrant soft silky feel that is immediately apparent to the user.

Alopecia is a skin condition that results in the loss of hair on the scalp and elsewhere. It usually starts with one or more small, round, smooth patches and occurs in males and females of all ages. Loss of hair in one or several small spots is common, but it is possible to lose all scalp hair (alopecia totalis), or every hair on the body (alopecia universalis), which is rare.

The skin condition, rosacea is of an unknown origin. It usually affects the middle third of the face causing skin redness, prominent vascularization, papules, pustules and swelling, as well as a predisposition to flushing and blushing. However, rosacea can also occur on other parts of the body including the chest, neck, back, or scalp. The blood vessels near the skin dilate and become more visible there through, resulting in telangiectasia. The resulting papules and pustules resemble teenage acne and are frequently mistaken for the same. Unlike acne, rosacea does not have blackheads or whiteheads. Rosacea, however, can occur in all age groups and in both sexes, where it tends to be more frequent in women but more severe in men. The flushing and blushing regions of the face are affected by rosacea. Emotional factors such as anxiety, embarrassment, or stress may evoke or aggravate rosacea. In addition, a flare-up may be caused by environmental or climate variances, and UV exposure is known to aggravate rosacea. Furthermore, diet is also known to aggravate rosacea. Spicy foods, alcoholic beverages, hot beverages, and smoking are known to cause flare-ups. Rosacea is not only an aesthetic complication. Rosacea is a chronic disease that has rarely been documented to reverse its progression. If untreated, the condition worsens and spreads. Untreated rosacea may cause a disfiguring nose condition called rhinophyma, which is characterized by a bulbous, red nose and inflamed cheeks. Severe rhinophyma may require surgery, an invasive procedure that may be avoided by timely treatment. Another problem of advanced rosacea is ocular. Persons afflicted with rosacea may experience conjunctivitis, a burning and grittiness of the eyes. If untreated, it may lead to serious complications such as rosacea keratitis, which damages the cornea and may impair vision.

Other skin conditions may include dry/chapped skin.

Burns involve a type of skin integrity rupture. Burns represent one of the most painful processes that can be established in this tissue, needing the establishment of a coordinated therapy to help its recovery and pain treatment. Burns can be caused by several factors, among which include, exposure to high or low temperatures, exposure to chemical compounds, by electricity, by exposure to radiation and mechanical friction, and the like. Burn severity and its risk are evaluated according to the amount of affected tissue and depth reached. The amount of affected tissue is represented by the percentage of burned corporeal surface (BCS). In this type of evaluation, burns can be divided into small, moderate, large, or massive burns, where regions inferior to 15% of BCS, from 15% up to 49% of BCS, from 50% up to 69% of BCS and over 70% of BCS, respectively. The extension of the affected area is determined through Lund-Browder scheme, which takes into consideration the burn proportion, in accordance with the age of the burned patient. Another rule that is most used for determining the extension of the affected area is that known as Wallace Rule or Rule of Nines, a technique less efficient than the foregoing, however, easy to memorize, being very much employed in emergency cases. This rule applies a value equaling nine or nine multiples to the affected parts, being 9% for each superior member, 9% for the head, 18% for each inferior member, 18% for each torso face and 1% for the genitalia.

The classification as first, second and third degree corresponds to burn depth. The first-degree injury corresponds to the burn that affects the skin most external layer (epidermis), not producing hemodynamic alterations, however the affected region is found hyperemic in absence of blisters or phlyctenae. This type of injury can be observed in erythemae resulting from sunrays or heated water. The second-degree injury affects either the epidermis as part of the dermis and is mainly characterized by the formation of blisters or phlyctenae, as those resulting from scalding or thermal injury resulting from overheated liquid. The third-degree injury endangers the totality of skin layers (epidermis and dermis) and, in many cases, can affect other tissues, as the subcutaneous cellular tissue, muscular tissue and bone tissue. Third-degree burns are considered as the most severe of all thermal injuries, producing deforming injuries. For being deeper, it eliminates the nerve endings responsible for shooting the painful message. These types of burns need transplanting for recomposing destroyed tissues, since the structures and organelles necessary for the natural recovery process, were eliminated. Since burns are wounds that involve the skin, they develop the afore-mentioned complex process of regeneration and recomposition of injured tissue. The speed or grade of re-epithelization of the affected region is dependent upon the size of the region affected by the burn; the rate or grade of re-epithelization is slower as the area involved is larger, considerably increasing the recovery time, when the injuries start to cover a body surface over 10% or 15%.

Immediately after the burn trauma, an inflammatory process develops wherein various agents are delivered, resulting in the deposition and activation of fibrins and platelets on the wound surface. A matrix rich in organic material is yielded, able to enclose bacteria and other strange substances, which frequently aggravates the case, due to sepsis that can follow trauma. During this inflammatory process, a great quantity of exudates crops out of the burned region, leading the patient to an intense loss of liquids, which, depending on the burn extension and depth, can cause a severe dehydration case. The inflammatory process extends to adjacent tissues, a factor that endangers the functions of these tissues initially intact. Extensive and deep burns cause alterations that are extended far beyond the affected local, such as anatomic, metabolic, physiological, endocrinology and immune alterations, requiring special care. Significant fluid losses, delivery of inflammatory multi-mediators and contamination by bacteria, occur. When disseminated in central organs through circulation, bacteria and inflammatory mediators can cause cardiac endangerment, failure of gastrointestinal mucous integrity and in extreme cases, multi-organic failure.

Hemodynamic alterations that occur after severe thermal injuries include decrease of cardiac output and reduced volume of circulating plasma, contributing all to a hypovolemic shock. Inflammatory mediators (including cytokines, prostaglandin, nitric oxide, and superoxide ions) have been implicated in causing further damage to tissues. It is believed that despite local benefit, such mediators induce undesirable effects when reaching significantly high levels. As an example, a greater damage to tissues can be caused by delivery of proteolytic enzymes and superoxide ions of macrophages and activated leucocytes.

Thus, burns are skin conditions that develop unbalance in a series of natural organic mechanisms, not limited to endangered tissues only, but involve numerous organs that can be affected. Additionally, large thermal injuries induce a sharp increase in basal metabolic rate. Large nitrogen corporeal losses, observed in burned patients, mainly occur due to protein exudation through burned skin and also by the fact that, under such catabolic stress situation, corporeal proteins can become the metabolic substrate used for production of 15 to 20% of total energy required by the organism. Further to these abnormalities, hormonal levels change with an increase in catecholamines, cortisol and glucagons, in the presence of normal or slightly increased levels of insulin. These hormonal alterations promote increase of proteolysis and lipolysis. Thus, the entire complex process is characterized by imbalance. The quick recovery of the skin of a burned mammal is of utmost importance for recovery of his normal organic functions. In an embodiment lidocaine is added to the oil mixture and heated during processing until there is a single-phase fluid. Burn victims have an immediate inflammatory response to stop further damage. When an anti-inflammatory oil is applied, blood flow increases and can cause sharp pain as vascular blood pressure expands damaged vesicles. A low level of lidocaine (for example, about 0.8 wt %) is added to the composition of the present disclosure to mitigate the pain and allow blood flow to resume to the damaged area.

To minimize scarring the composition of the present disclosure is applied to wounds, burns or skin conditions during healing after the wound is closed.

Embodiments of the compositions of the present disclosure exhibit “cosmetically elegant” non-sticky surface skin feel, while at the same time are capable of transferring important ingredients into the targeted layers of the skin. By “cosmetically elegant,” it is meant that the composition provides silky smooth skin feel, after rub-in, with no malodor and no perceived greasiness. Rub-in is mechanically accomplished in seconds, but the actual absorption of liquids takes about 60 seconds. This is intentional, to “signal” a lotion-like experience.

Since the mixture is anhydrous and since lotions comprising the compositions of the present disclosure are mostly water, larger gram amounts are dispensed to achieve sufficient functional ingredient concentration. In an embodiment, the package is an airless pump with, for example, a 0.2 ml micro-dose per squirt (SR Packaging, Taiwan). One squirt per arm is usually sufficient. Users quickly calibrate their dose depending on how dry their skin is. Faces, typically, require one squirt (as a nighttime hydration protocol); legs may require two squirts per leg.

Various treatments may be employed. Skin surfaces of the most concern tend to be those not typically covered by clothing such as facial skin surfaces, hand and arm skin surfaces, foot and leg skin surfaces, and neck and chest skin surfaces. In particular, facial skin surfaces, including the forehead, perioral, chin, periorbital, nose, and/or cheek skin surfaces, may be treated with the compositions described herein. The composition should not be placed directly into the eye.

The treatment method may include applying the composition(s) to a previously identified area of skin in need of treatment for reducing the tendency of scarring or in inhibiting or preventing scarring or other skin conditions. The composition(s) may be applied at least once a day, twice a day, or on a more frequent daily basis, during a treatment period, as prescribed by the physician. When applied twice daily, the first and second applications are separated by at least 1 to 12 hours. In an embodiment, the composition(s) may be applied in the morning and/or in the evening before bed. In another embodiment, the application precedes a normal shower. Any excess oil washes off, leaving the silky-smooth finish and hydrated skin.

The treatment period is of sufficient time to provide an improvement in the appearance of the skin wherein scarring is invisible to the naked eye or where there is inhibition or prevention of scarring or where the scar tissue is minimal in size so that it is hardly noticed by a third party with the naked eye. Scarring is not eliminated, because collagen ingress is part of the wound closure process. What is important is to keep the skin hydrated so as not to “signal” for more collagen production. Elimination of the body's recognition of the loss of barrier function is critical to minimizing collagen ingress (visible scarring). Large areas of skin can be treated, but the practical reality is the larger the wound, the greater the scarring. Small areas, like cosmetic surgery, acne, psoriasis, rosatia etc., will have almost invisible scarring. The treatment period may be at least 1 week, and in some embodiments the treatment period may last about 4 weeks, 8 weeks, or 12 weeks or more. In certain embodiments, the treatment period will extend over multiple months (i.e., 3-12 months) or multiple years as wounds remodel. In one embodiment the composition is applied at least once a day during a treatment period of at least 4 weeks, 8 weeks, or 12 weeks. In one embodiment the composition is applied twice a day during a treatment period of at least 4 weeks, 8 weeks, or 12 weeks. In another embodiment, a chronic wound is closed using anhydrous collagen products and the composition is applied immediately after closing until the wound is completely remodeled. As used herein, the term “chronic wound” refers to a wound that has not healed within a normal time period for healing in an otherwise healthy subject. Chronic wounds may be those that do not heal because of the health of the subject, for example, where the subject has poor circulation or a disease such as diabetes, or where the subject is on a medication that inhibits the normal healing process. Healing may also be impaired by the presence of infection, such as a bacterial, fungal, or parasitic infection. In some instances, a chronic wound may remain unhealed for weeks, months or even years. Examples of chronic wounds include but are not limited to, diabetic ulcers, pressure sores, vascular insufficiency wounds and tropical ulcers.

Scarring inhibiting effective amounts of the composition of the present disclosure are applied topically to the area to be treated. The dose varies with the individual and the skin condition. In an embodiment, the dose administered to patients are subjects is that dose that provides as much of the compositions as the skin will absorb. That is, dry skin will require more composition than hydrated skin, and larger wounds will require more composition than smaller wounds. An excess amount of the compositions on the surface of the skin can turn rancid. A silky smooth, odorless finish remains on the skin for 24 hours. The dosage regimen for treating skin conditions, burns and/or wounds is selected in accordance with a variety of factors, including the age, weight, sex, and medical condition of the patient, the severity of the condition, pharmacological considerations such as the activity, efficacy, pharmacokinetic and toxicology profiles of the particular composition used. A physician determines the dosage regimen.

The doses may be administered in single or divided applications. The doses may be administered once, or application may be repeated. Application may be repeated weekly until skin and/or wound healing is promoted with little, or no scarring. Doses may be applied 1-7 days apart, or more. In the case of a chronic skin condition or wound, repeat applications may be made, for example, one or more times per day, weekly, or bi-weekly, or monthly or in any other frequency for example, if and when healing slows or is stalled. For some indications, more frequent dosing such as hourly application may be employed.

Signs that there is excessive composition usage are bright pink wounds from excess vascular development. As wounds first start to close, the body naturally grows new vascular capacity to bring nutrients needed for new tissue growth. Once the wound is closed and the need for new tissue has passed, the body naturally consumes the excess vascularity. During this transition, the flesh changes from pink (excess vascularity) to normal color (maintenance vascularity). Doctors and patients will see this change and adjust their consumption accordingly.

When the wound or skin condition is fully granulated and the wound has closed, the present composition is applied topically to the situs of the wound bed surface, or surface of the burn or skin condition. One of ordinary skill in the art, such as a physician can make that determination based upon various factors, such as the severity of the wound, burn or the skin condition, the age of the patient, the health of the patient and the size of the wound, burn or skin condition including the depth thereof and the like. It covers completely the situs of the wound, burn or skin condition and the intact skin adjacent to the area, such as the periwound, in an amount effective to protect the situs from infection and/or excess ROS species and/or to retard the growth of pathogens, such as bacteria. A therapeutically effective amount of the composition is applied to the situs of the burn, wound or skin condition. Further, the composition covers completely area adjacent to the situs of the wound, burn or skin condition, such as the periwound, for example, or in another embodiment, the area that borders or is adjacent to and in close proximity to the situs, for example, an area up to 6 inches in diameter around the situs. In an embodiment, the dose is that dose where the user does not perceive greasiness, i.e., sufficient amount to saturate the skin with composition. Users want to obtain as much composition as possible up to the point of cosmetic inelegance, i.e., greasiness, malodor, grittiness, in short, until it feels uncomfortable. The physician could prescribe the maximum amount that will avoid cosmetic inelegance. Greasiness is a signal that the skin cannot absorb any more lipids. When the skin is just recovering from insult, a large dose is not greasy. As the skin heals, healing skin needs less stimulation and the same dose that was previously appropriate is now greasy. This is a normal and easy determination by the user, particularly with the micro-dose package. Periodically, the wound is inspected and, if the skilled medical physician so determines, the composition is reapplied as before. Again, the frequency of removing and reapplying the composition is determined by the physician, patient, or care giver, and is dependent upon many factors, including the health and age of the patient, the size of the wound or skin condition or burn and the drainage from the wound, skin condition or burn, the sex of the patient, mental competence, and the like.

In an embodiment, the thickness of the amount applied ranges from about 50 μm to about 5 mm. In other embodiments, the thickness of the amount applied ranges from about 50 μm to about 2 mm, or about 50 μm to about 1 mm, or about 50 μm to about 750 μm or about 50 μm to about 500 μm, or about 50 μm to about 300 μm or about 50 μm to about 100 μm or about 100 μm to about 2 mm, or about 100 μm to about 1 mm, or about 100 μm to about 750 μm, or about 100 μm to about 500 μm, or about 100 μm to about 300 μm, or about 0.5 mm to about 5 mm, or about 0.75 mm to about 5 mm, or about 1 mm to about 5 mm, or about 1.5 mm to about 5 mm, or about 2 mm to about 5 mm, or about 3 mm to about 5 mm, or about 0.5 mm to about 3 mm, or about 0.75 mm to about 3 mm, or about 1 mm to about 3 mm, or about 1.5 mm to about 3 mm, or about 2 mm to about 3 mm. But the thickness of application changes in real time as most of the composition is quickly absorbed and the residual thickness is hard to measure.

The process of removing the composition (simple water rinse or with soap and water) and replacing with a fresh composition is repeated until the wound has healed and remodeling is complete, and the size of the scar tissue is reduced and or invisible to the naked eye. Merely rinsing the skin with water and drying the site removes excess composition. The residual wax/saturated fat film is flexible and is now part of the skin barrier structure.

In an embodiment, besides applying the composition of the present disclosure when the skin is closed, in another embodiment, when the wound or burn or skin condition first appears, the topical composition described herein is applied to an area proximate to the skin condition or wound or burn but not on the skin condition or wound or burn. It is placed on a proximate area which is about 2 times the diameter of the size of the wound, burn or skin condition up to about six inches from the skin condition or wound or burn and then over the entire area approximate thereto (e.g., the periwound). For example, if the wound or skin condition or burn is on a limb, finger, or toe, then the skin composition is applied topically over substantially the remainder of the limb, finger, or toe, respectfully. If it is on the shoulder, back or stomach, then the composition of the present disclosure is applied topically over substantially the rest of the shoulder, back, or stomach, respectively. The function thereof is to exfoliate old dead and dying skin in the proximate area of the wound or skin condition to bring new, viable skin to the surface. Without wishing to be bound, it is believed that as this composition is absorbed, the anti-inflammatory omega-3 oils help reduce inflammation in the vascular system. The vascular system, particularly the microcapillaries, relaxes to allow a pulse of greater blood flow throughout the skin (perfusion pulse). The temporary increased blood flow helps heal the wound more rapidly and reduce scarring.

In another embodiment, the composition is used to treat the wound, skin condition, or burn when the wound or burn first occurs or when the skin condition first occurs or is first noticed. In this embodiment, the composition contains an analgesic, which is applied topically to the wound, skin condition or burn. The treatment regimen and the dosage are determined by the physician based upon various factors, such as the severity of the wound, burn or the skin condition, the age of the patient, the health of the patient and the size of the wound, burn or skin condition including the depth thereof and the like. Treatment of the wound, burn or skin condition is continued until the wound or skin condition, or burn is fully granulated. Then, the treatment regimen is as described above.

In conclusion, the composition of the present disclosure
1. does not have that greasy feeling, but feels like a smooth lotion applied to the skin;
2. moisturizes in the absence of added moisture (via reducing TEWL);
3. has little or no off odors (like rancid fat (oxidized) and has little, if any of the rancid “fishy” smell (which is due to trimethylamine);
4. penetrates the epidermis during rub-in;
5. is silky-smooth” post-rub-in skin feel;
6. has long-lasting skin cosmetically elegant feel;
7. has little or no stickiness.

Further, formulations of the present disclosure do not scar or minimize scarring after application of the formulation described herein to the wound, burn or skin condition.

The following examples further illustrate the teachings of the present disclosure. It is to be understood that Counter examples listed hereinbelow are not considered prior art. The Counter examples vary a variable, illustrating an adverse consequence when the variable is not within the teachings of the present disclosure. Further, unless indicated to the contrary, the percentages listed are in weight %.

### Example 1

Omeza Lotion K7P, also identified as K7p, is comprised of the following composition:
OMEZA LOTION K7P

| | |
|---|---|
| MCT oil | 69.70% |
| Monolaurin | 10.00% |
| Cetyl esters NF | 5.84% |
| Ascorbyl palmitate | 0.50% |
| Hemp oil | 2.41% |
| Cod liver oil | 2.36% |
| RBD palm oil | 2.78% |
| Rice bran wax | 5.71% |
| Squalene/Vitamin E | 0.20% |
| Colloidal oatmeal | 0.10% |
| Coconut FFA | 0.05% |
| Virgin coconut oil | 0.35% |
| Total | 100.00% |

PUFA Ratio: 1.00
Sum Omega3: 1.00
Penetration Ratio: 8.91

The Example 1 batch size is 400 g. All ingredients, except colloidal oatmeal are heated together until clear (˜180° F.). Colloidal Oatmeal is added, and the hot mixture is transferred to a mixing bowl, Kitchen Aid Mixer, Model Number KSM7586 and stirred @“1” (out of “8”).

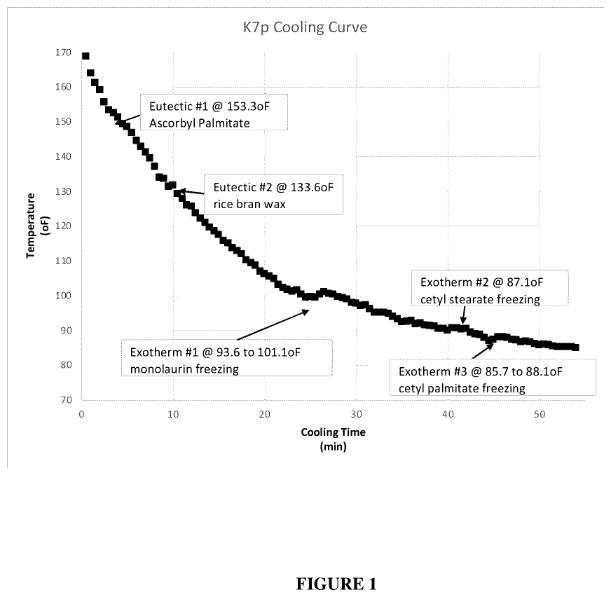

The product temperature is measured every 30 seconds with an infrared temperature “gun”. An inert gas blanket was maintained throughout cooling. The cooling curve for K7p is shown in FIG. 1. Segments of the response curve were fit to a linear trendline, and both the Coefficient of Determination (R²) and the best fit line were calculated. The best fit curve was defined as the maximum R² for any set of contiguous data points. When the slope of the line changed, a new best fit line was calculated. The inflection point between adjacent best fit lines is the cooling time where each best fit line predicted the same temperature.
1. Eutectic #1 (153.3° F.) is the freezing point of ascorbyl palmitate.
2. Eutectic #2 (133.6° F.) is the freezing point of rice bran wax.
3. Exotherm #1 (begins @93.6° F.; ends @101.1° F.) is the freezing of monolaurin and the exothermic formation of nanoparticle liquid capsule (NLC).
4. Exotherm #2 (87.1° F.) is the exothermic freezing of cetyl stearate (one of the esters in Cetyl Esters NF).
5. Exotherm #3 (begins @85.7° F. and ends @88.1° F.) is the exothermic freezing of cetyl palmitate (one of the esters in Cetyl Esters NF).

### Explanation of EXAMPLE 1

K7p is heated until it is a single-phase fluid. Then a small amount of colloidal oatmeal is added. The fluid is stirred at a low shear rate but not so much shear that gas is mixed into the fluid. As cooling progresses, ascorbyl palmitate precipitates, and then rice bran wax crystallizes. The rice bran wax crystals are needle like (orthorhombic).

The fluid is now a two-phase fluid, solid and liquid. Monolaurin precipitates and forms nanoparticle lipid capsules (NLC). Temperature increases (exothermic phase change) as the NLC structure forms from the single-phase liquid. The colloidal oatmeal, and liquid lipids (primarily MCT, hemp oil, cod liver oil and cetyl esters) are encapsulated by the nonionic surfactant, monolaurin, in an anhydrous single-phase fluid. The ascorbyl palmitate and rice bran wax crystals precipitate on the outer surface of the monolaurin NLC (Pickering Effect). With sufficient monolaurin, all the fluid is encapsulated by freezing monolaurin surfactant.

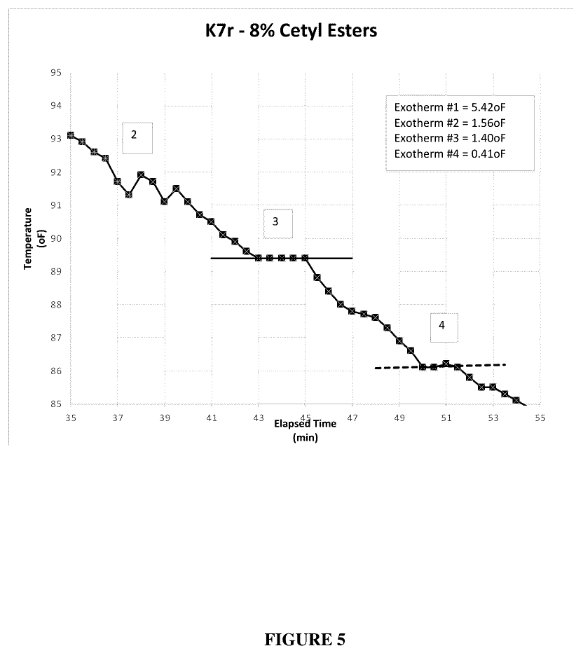

The weight ratio of monolaurin to cetyl esters is about 10.0 to about 5.84. More importantly, this is about a 3:1 mole ratio. Monolaurin has a molecular weight of 274.4. Cetyl Esters NF is a mixture primarily of three esters (cetyl stearate; MW=508.9), cetyl palmitate; MW=480.8, and cetyl myristate MW=452.8). (Cetyl Esters NF is sometimes called just cetyl palmitate because the average molecular weight of Cetyl Esters NF˜480.7.) As shown in FIG. 5, which is exemplary for the behavior of the compositions of the present disclosure after Exotherm #1 is complete (the chart is a late stage subset of the cooling data set to better illustrate Exotherms 2, 3 & 4), each of these wax esters (Exotherms, 2, 3 & 4) precipitates at a slightly different temperature. FIG. 5 shows the cooling response of a composition with 8% cetyl esters (not 5.84%), to better show the exotherms of each cetyl ester. The cooling rate is intentionally slow to allow each ester to complete its precipitation before the next wax ester starts its precipitation. The net effect is to form distinct ester layers. Skilled artisans will recognize that the measured exotherm will increase with higher cetyl ester concentration and vice versa.

Cooling continues until the three lipid esters in Cetyl Esters NF freeze one by one inside the NLC (shown schematically in FIG. 16). Cetyl stearate freezes @57° C.; cetyl palmitate freezes @54° C.; cetyl myristate freezes @50° C. Ester freezing is slightly exothermic.

In K7p, the nanoparticles are formed from a single miscible fluid, surfactant, and modest shear. There is no water; there is no solvent; there is no residue; there is no active ingredient partition between the two immiscible fluids; everything liquid is encapsulated in the NLC. Rice bran wax (RBW) needle crystals have a lower density than the molten liquids and float away during NLC formation and precipitate in a random array on the NLC surface (due to the Pickering Effect). High density solids (“cargo”), like colloidal oatmeal, do not float to the surface and are captured within the NLC during NLC formation.

NLC structures are notoriously weak and fail under shear. As sketched in FIG. 16, the monolaurin “skin” is reinforced by the sequential internal layering of the cetyl esters and by the external reinforcing layer of RBW needle-like crystals. In other words, the skin is sandwiched between inner wax layers and an outer wax layer . . . a spherical “Oreo” cookie. This structure is previously unknown.

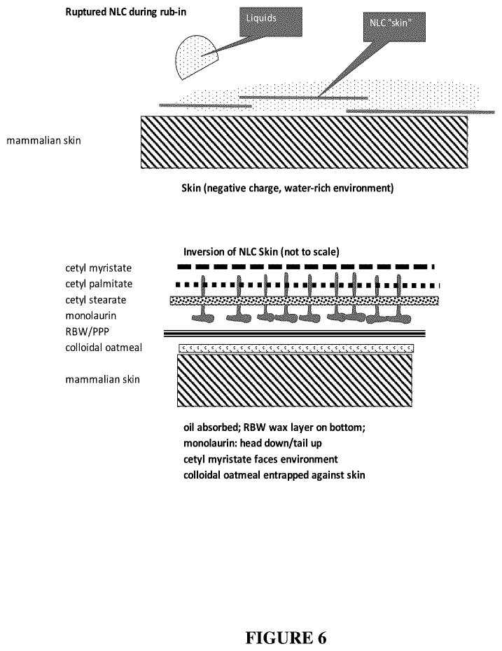

The RBW needles form first and are rejected (by density difference) when the NLC skin forms. The cetyl esters are liquids, not yet solid, when the NLC forms and thus are encapsulated within each NLC sphere. With gentle mixing (to prevent unwanted internal accumulation of solids), each cetyl ester freezes (longer chain first; shorter chain last) at the coldest point on the interior surface of the NLC. Heat is lost at the surface of the NLC, such that the inner surface of the NLC is the cetyl ester freezing point. After cetyl ester freezing, gently mixed cooling continues until the RBW needles precipitate on available surfaces (i.e., the NLC outer surface). The net effect of this programmed structure formation creates a shelf stable NLC that is “unstable” when rubbed into skin, that is, the liquids are released (& absorbed) and the structured NLC composite “skin” bonds to the mammalian skin to form a moisture occlusive and oxygen occlusive layer over the mammalian skin with the lowest molecular weight cetyl ester (most silky smooth) on the outside facing the atmosphere. (FIG. 6)

K7p formed according to this procedure is “instantly” absorbed by the patient's skin (˜15 seconds) when applied thereto; after absorption, the skin feel is silky-smooth. There is no odor. The product has the texture of a slightly granular cold cream. Users report their skin feels hydrated when the composition of Example 1 is applied thereto.

### Example 2

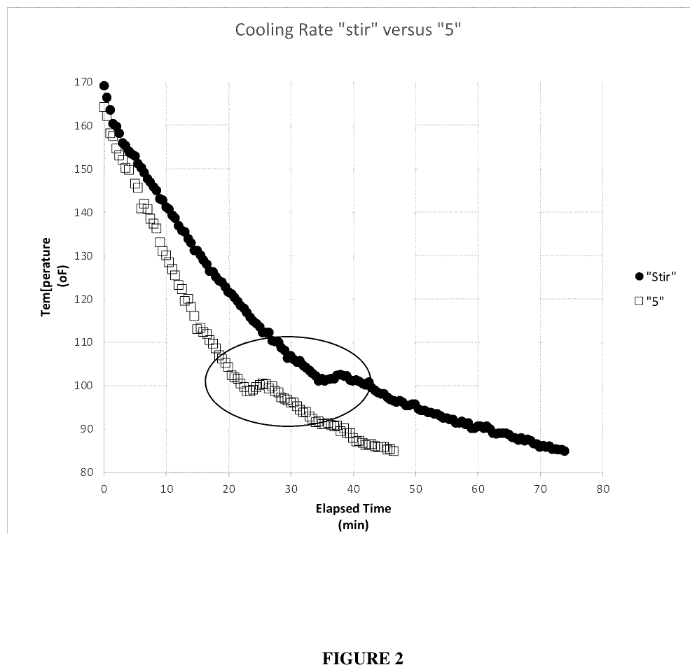

In Example 2, Example 1 compositions (K7p) were sheared at two different mixer speeds with a wire whisk, “stir” (100 rpm), the slowest possible, and “5” (200 rpm) of “8”. Otherwise, the processing was the same as Example 1. The cooling lines to 85° F. were determined (and graphically depicted in FIG. 2).

After cooling to ambient overnight, a 30 ml glass jar of Example 2 was placed in an incubator @40° C. for 15 hours. The jar was examined for separated fluid. When mixed @“5”, there was separation; there was no separation @“stir”. There is no difference in skin feel or odor between the compositions subjected to the two mixing protocols described hereinabove.

### Explanation of EXAMPLE 2

Example 2 was conducted to determine whether shear was a critical factor or not. The issue investigated was whether Exotherm #1 always occurred or whether it was an artifact of shear. The zone captured by the ellipse in FIG. 2 shows that Exotherm #1 was essentially the same with low shear or medium shear.

Moreover, mixing @“5” with a wire whisk incorporated some inert gas into the fluid. Gas bubbles incorporate extra surface area in the single-phase fluid as Exotherm #1 forms NLC. The mass of monolaurin does not change, so the monolaurin partitions between the NLC surface and the gas bubble surface.

The net effect is to reduce the concentration of surfactant around each NLC particle. The particles leak in the high temperature stress test because there was not enough surfactant to keep the NLC intact.

In Example 1, the mixing was reduced to “1”, marginally above Example 2 @“stir”. This small change stopped gas incorporation into the composition and still kept colloidal oatmeal concentration uniform throughout the batch. Example 1 is stable. Mixing speed is not important for positive-benefit NLC formation; mixing is important for NOT incorporating negative-benefit gas. Minimum mixing with a paddle attachment is ideal.

### Counter Example 1

OMEZA GEL CS1D

| | |
|---|---|
| MCT Oil | 52.09% |
| Monolaurin | 10.00% |
| Cetyl esters NF | 2.00% |
| Virgin coconut oil | 0.33% |
| RBD palm oil | 7.12% |
| Ascorbyl palmitate | 0.50% |
| Hemp oil | 12.00% |
| Cod liver oil | 16.21% |
| Colloidal oatmeal | 0.25% |
| Total | 100% |

PUFA Ratio: 0.75
Sum Omega3: 6.01
Penetration ratio: 6.69

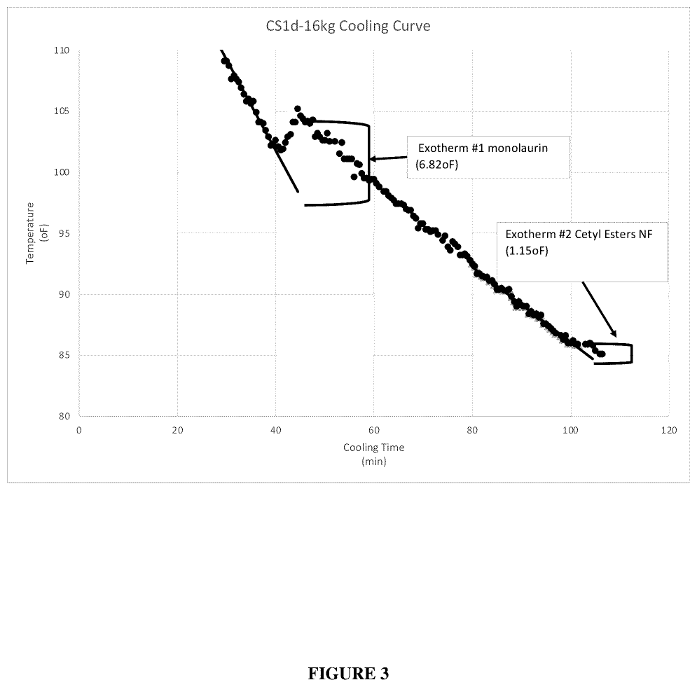

A 16 kg batch of CS1d is prepared using the Example 1 method, except that after melting, the batch was split into 8×2,000 g mixing bowls and mixed @“stir” (with wire whisk @100 rpm). The cooling curve is analyzed and is depicted in FIG. 3. There are two distinct exotherms.

### Explanation of COUNTER EXAMPLE 1

Normally, a differential scanning calorimeter (DSC) is used to measure changes in enthalpy. In a shearing environment, the DSC technique does not work. The best fit line just before Exotherm #1 and Exotherm #2 is extrapolated to the peak temperature measured after each exotherm is initiated. The difference in the extrapolated (and calculated) beginning point and the observed maximum temperature is defined as the maximum heat difference. In COUNTER EXAMPLE 1, Exotherm #1 rose 6.82° F.; the sum of Cetyl Esters NF temperature rise (Exotherm #2) rose 1.15° F., compared to 3.37° F., in Example 3 (sum of Exotherms #2, 3, 4). See below. This is almost a 3× rise. The absolute value of Exotherm #2 is small because the cetyl esters concentration is 2% versus 5.84% in Example 1.

The absolute magnitude of the temperature rise is the sum of the exotherm minus the heat lost in the processing. A direct comparison between a 400 g batch and a 16,000 g batch is not possible because the cooling kettles have very different heat transfer coefficients. What is important is the relative size of the temperature rise compared to a base formulation. The objective is to maximize the temperature rise, an indirect indicator of how close the system came to achieving the lowest free energy state (most stable).

After conducting COUNTER EXAMPLE 1(@16,000 g), studies were fixed at 400 g per study to take the size of the reactor out of the equation.

CS1d has 2% Cetyl Esters NF; K7r of EXAMPLE 3 has 8% Cetyl Esters NF. Exotherm #2 is directly related to the mass of Cetyl Esters NF. With Cetyl Esters NF levels below 4%, the cooling curve technique is not sensitive enough to pick up the three possible exotherms (Exotherms #3, 4 & 5) of the individual ester chain lengths.

CS1d was analyzed for % colloidal oatmeal. The variation did not meet the Quality system requirement. Even though CS1d formed nanoparticles (Exotherm #1), they were not strong enough to hold the oatmeal cargo because 2% Cetyl Esters NF does not strengthen the nanoparticle sufficiently to hold solid cargo.

CS1d is remelted, destroying the NLC, and remixed just prior to filling. Filling is only into single use packages that freeze 6 minutes after filling. This resolved the oatmeal variation but destroyed the nanoparticles in the process.

### Example 3

K7r is prepared using the procedure of Example 1. The composition is as follows:
Omeza Lotion K7r
OTC Skin Protectant Lotion

| | |
|---|---|
| MCT oil | 67.37% |
| Monolaurin | 10.00% |
| Cetyl Esters NF | 7.98% |
| Ascorbyl palmitate | 0.49% |
| Hemp oil | 2.40% |
| Cod liver oil | 2.35% |
| RBD palm oil | 2.77% |
| Rice bran wax | 5.69% |
| Squalene/Vitamin E | 0.20% |
| Coconut FFA | 0.05% |
| Virgin coconut oil | 0.35% |
| Colloidal oatmeal | 0.35% |
| Total | 100.00% |

PUFA Ratio: 1
Sum Omega3: 1.00
Penetration Ratio: 8.64

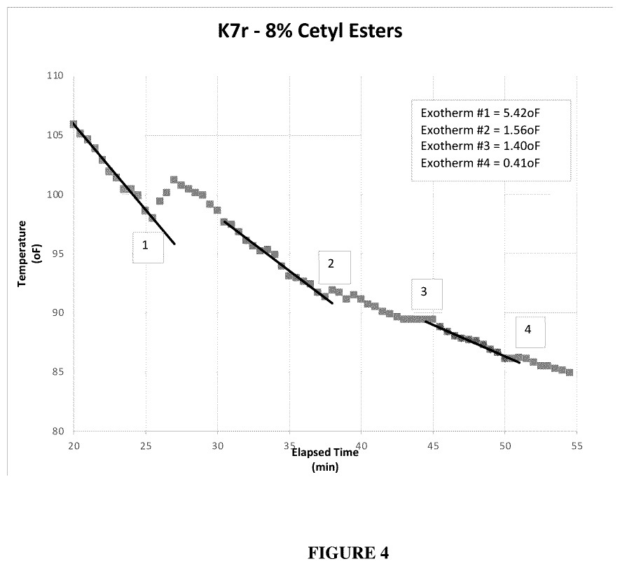

K7r is stirred @“4” with a wire whisk until 95° F. (after Exotherm #1 is complete). Stirring is reduced to the minimum possible (“stir”), producing Exotherms #2, 3 & 4). The cooling curve is depicted in FIG. 4.

K7r has excellent stability, hydration, skin feel and odor. It has 8% Cetyl Esters NF (FIG. 4).

K7r has 4 distinct freezing exotherms: one for monolaurin, one for cetyl stearate, one for cetyl palmitate and one for cetyl myristate. The size of each exotherm was calculated using the Example 3 technique.

Exotherm #3 and Exotherm #4 have a constant temperature for 2 minutes of cooling. This is the tie-line pattern for crystallization, not gelation. FIG. 4 and FIG. 5 are the same data set; FIG. 5 illustrates the response of a subset of all the data so that small differences are easier to visualize.

K7r has the following characteristics:
1. Oil absorption is complete after 15 seconds.
2. During oil absorption, users describe an oil-in-water, lotion-like application experience.
3. After oil absorption, there is no greasy feeling, and the skin surface is silky smooth.
4. There is no odor.
5. TEWL is reduced significantly.
6. K7r is used as a nighttime facial hydration product or a daytime non-facial moisturizer.
7. Over time, facial skin pore size is reduced.
8. Skin is described as smoother and well hydrated after application thereto for 30 days.

### Explanation of EXAMPLE 3

Ascorbyl palmitate and rice bran wax form crystals before NLC formation. When NLCs form (Exotherm #1) lipid liquids surround the “cargo” of colloidal oatmeal. The 10% w/w monolaurin surfactant spreads evenly over the entire surface of each NLC particle. Surface tension forces bend the NLC surface into a sphere (lowest energy state). Specific gravity declines. Low density solids (i.e., RBW needles) float out of the forming-NLC and come to rest on the NLC surface.

When the ester exotherms occur, the internal volume of the encapsulated fluid increases as molten liquids turn to solids. Specific gravity decreases, and monolaurin concentration on the surface of the “spheres” equalizes as the surfactant forms a sphere. Internal produced solids expand and increase internal pressure. Cetyl ester waxes are substantially linear and form along the inside surface of the monolaurin skin. This precipitation process is abetted by shear. Without shear, the precipitation can be non-uniform; with shear, the inner surface of the NLC is a constant temperature that freezes each cetyl ester at its freezing point. There is an optimum monolaurin to cetyl esters ratio of about 1:1 to about 4:1. Eventually, a 3:1 mole ratio was experimentally determined to be optimal (1.71:1 w/w).

There are 6 steps to NLC formation:
1. Solid crystals form (Eutectic 1; Eutectic #2) first.
2. Colloidal oatmeal is added.
3. Monolaurin forms an NLC around the liquid lipids and cargo crystals—Exotherm #1
a. Shear is minimal (“stir” (100 rpm) with a paddle attachment).
4. Cetyl Esters NF contain 3 primary molecules all with slightly different melting points. The exotherms #2, 3, 4 are different, as shown in the table below based on FIG. 5:
a. Cetyl stearate—Exotherm #2 (46.3% of total temperature rise)
b. Cetyl palmitate—Exotherm #3 (41.5%)
c. Cetyl myristate—Exotherm #4 (12.2%)
5. Cetyl Stearate forms a uniform solid that coats the inside of the NLC. Liquid lipids and cargo crystals are trapped inside the NLC.
a. The continuous asymmetric rotation of the NLC with gentle mixing forces the heavy colloidal oatmeal to be suspended within each NLC sphere.
6. The coldest part of each NLC is the surface where cooling takes place.
a. With slow cooling, cetyl stearate and then cetyl palmitate precipitate as two wax layers on the cold interior surface of each NLC, entrapping the colloidal oatmeal in place encapsulated by the monolaurin aliphatic tail.
b. With continued slow cooling, cetyl myristate precipitates as a third wax layer on the cold interior of each NLC, forming layers, schematically shown in FIG. 16.

There are 5 steps to application and rub-in as shown in FIG. 6.
1. A dose of NLCs is rubbed onto the skin of the patient, rupturing each NLC and delivering the interior contents onto the intact skin surface.
a. Users describe the rub-in as “lotion-like.”
2. Waxes and liquid lipids are distributed over the water-rich, negatively charged mammalian skin.
a. The oils are absorbed in ˜15 seconds.
3. MCT drags the other liquid lipids through the stratum corneum, leaving free fatty acid, “cargo” and wax layers on the skin surface.
4. The structured NLC “carcass skin” has a lipophilic side (cetyl myristate on the atmosphere-facing side) and a hydrophilic side (head group of monolaurin nonionic surfactant on the skin-contact side).
a. The monolaurin head group is attracted to the “wet” skin, trapping the wax and cargo crystals between the skin and the layered wax structure.
5. The carcass inverts and forms a contiguous layer over the water-rich, negatively charged, intact skin surface.
a. The silky smooth cetyl myristate is on the touchable side (atmospheric side) of the layered structure.
b. The cargo is trapped between the cetyl esters top three layers and the monolaurin aliphatic tails.
c. Interspersed rice bran wax (RBW) layers from all the ruptured NLCs form an oxygen/moisture occlusive layer over mammalian skin (FIG. 6).
d. Previously protonated FFA dissolves in the skin moisture after rub-in and partitions into protonated FFA and unprotonated FFA (thus donating a H⁺ ion to the skin surface. The FFA partitioning helps buffer the skin pH slightly acidic. Slightly acidic skin pH makes MMP collagen-dissolving enzymes more active.

### Example 4

Using K7p prepared in Example 1, the effect of varying the concentrations of monolaurin, rice bran wax, MCT and cetyl esters was studied. The effect of varying the concentrations of monolaurin, rice bran wax, MCT, and Cetyl Esters NF is tabulated in the Table 1. Table 1 lists the changes to K7p. When a variation is introduced, MCT is adjusted to balance the formula. When MCT is varied, palm oil was adjusted accordingly, except for 75% MCT when palm oil, hemp oil and cod liver oil are adjusted to balance.

TABLE 1
(significant effects highlighted)

| | | as made | as made | aged | aged |
|---|---|---|---|---|---|
| | | sp. Gr. | viscosity | sp. Gr. | viscosity |
| MCT | 40% | 0.816 | 182,000 | 0.811 | 341,000 |
| | 50% | 0.844 | 214,000 | 0.839 | 270,000 |
| | 60% | 0.865 | 248,000 | 0.870 | 258,000 |
| | 69.7% | 0.874 | 99,400 | 0.881 | 419,000 |
| | 75% | 0.907 | 198,000 | 0.900 | 312,000 |
| RBW | 4% | 0.899 | 112,000 | 0.871 | 405,000 |
| | 5.71% | 0.874 | 99,400 | 0.881 | 419,000 |
| | 8% | 0.879 | 266,000 | 0.873 | 402,000 |
| monolaurin | 8% | 0.896 | 197,000 | 0.887 | 345,000 |
| | 10% | 0.874 | 99,400 | 0.881 | 419,000 |
| | 12% | 0.856 | 179,000 | 0.859 | 247,000 |
| Cetyl Esters | 4% | 0.893 | 131,000 | 0.875 | 476,000 |
| | 5.84% | 0.874 | 99,400 | 0.881 | 419,000 |
| K7r | 8% | 0.897 | 210,000 | 0.894 | 235,000 |

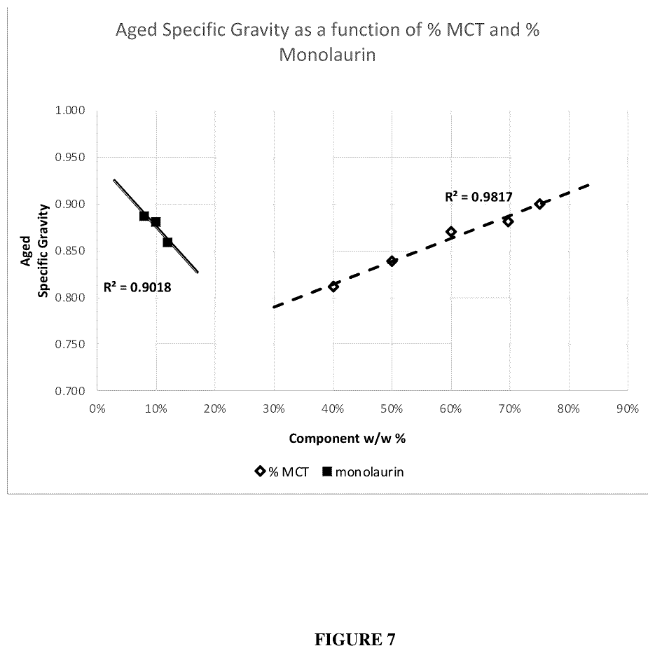

FIG. 7 shows that MCT increases aged specific gravity in direct proportion to the MCT concentration. Monolaurin does the opposite in inverse proportion to increasing monolaurin concentration.

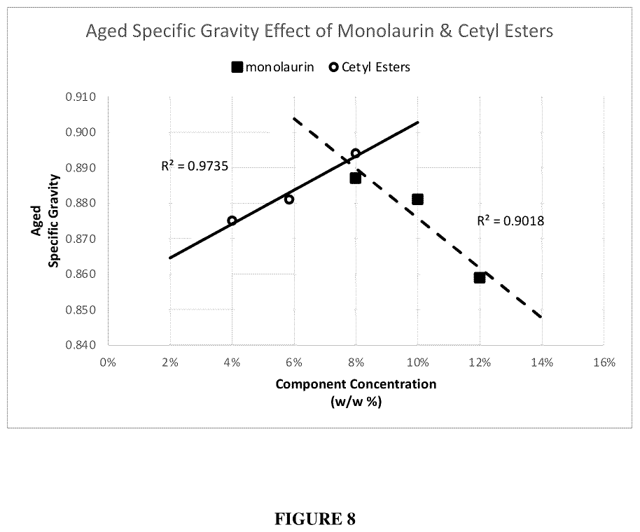

FIG. 8 shows that increasing cetyl esters directly increases aged specific gravity and that monolaurin does the opposite.

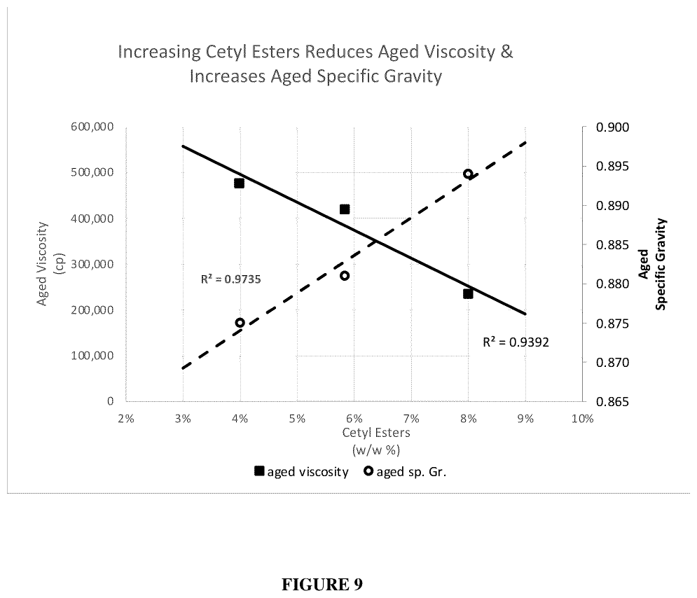

FIG. 9 shows that aged viscosity decreases directly with increasing cetyl esters concentration, while aged specific gravity increases directly with increasing cetyl esters concentration.

### Explanation of EXAMPLE 4

Monolaurin and capric/caprylic triglycerides (MCT) with high shear and rapid cooling in combination create the nanoparticles and two phases. This experiment shows that increasing the amount of monolaurin makes larger diameter nanoparticles, reducing packing density and lowering specific gravity. Larger nanoparticles hold more 0.931 specific gravity MCT (from MCT Certificate of Analysis, PG Chemicals, Cincinnati. OH), increasing specific gravity of the mixture thereof from ˜0.80 towards 0.91.

In FIG. 8, Table 1 monolaurin aged specific gravity data are superimposed with the effect of cetyl esters. Monolaurin decreases specific gravity; cetyl esters increases specific gravity. As described in Example 3, cetyl esters precipitate just under the monolaurin skin, strengthening the skin. The surfactant increases internal pressure (surface tension); internal crystal formation increases the contained volume (Exotherm #2) adding to the internal pressure on an individual nanoparticle. The nano particle changes shape from a perfect sphere to fill the void space between stacked spheres, thereby increasing specific gravity.

Viscosity reduction was a surprise. With the nanospheres packed more tightly together, it was expected that viscosity would increase, but viscosity decreased. The explanation for the drop in viscosity is provided in Example 10 after experiments on post cooling shear.

### Example 5 (Counter Example)

Using the procedure described herein in EXAMPLE 1, the following example is prepared:
Omeza Lotion K7f
OTC Skin Protectant Lotion

| | |
|---|---|
| MCT oil | 70.00% |
| Monolaurin | 10.00% |
| Cetyl Esters NF | 5.84% |
| Virgin coconut oil | 0.34% |
| Ascorbyl palmitate | 0.50% |
| Hemp oil | 2.41% |
| Cod liver oil | 2.36% |
| RBD palm oil | 3.00% |
| Rice bran wax | 5.45% |
| Colloidal oatmeal | 0.10% |
| Total | 100.00% |

PUFA Ratio: 1.00
Sum Omega3: 1.00
Penetration Ratio: 8.92

In Example 5, the importance of slow cooling during Exotherm 2, 3 & 4 is shown.

K7f was mixed @“4” (wire whisk) until the temperature of 125° F. was reached and then @“3” until the temperature of 85° F. was reached. After overnight quiescent cooling to 74° F., K7f was mixed @“stir” (wire whisk) for just 15 seconds.

There was bottom oil separation @40° C./15 hours. When the separated liquid returned to 25° C., the separated liquid froze to an opaque solid.

Aged viscosity was 291,000 cp; aged specific gravity was 0.877.

### Explanation of EXAMPLE 5

A small amount of shear (“stir/15 seconds”) after 75° F. ruptured fragile nano-liquid-capsules.

### Example 6 (Counter Example)

The following composition is prepared as described above:
Omeza Lotion K7i
OTC Skin Protectant Lotion

| | |
|---|---|
| MCT oil | 69.43% |
| Monolaurin | 10.00% |
| Cetyl Esters NF | 5.84% |
| Virgin coconut oil | 0.40% |
| Coconut FFA | 0.20% |
| Ascorbyl palmitate | 0.50% |
| Hemp oil | 2.41% |
| Cod liver oil | 2.36% |
| RBD palm oil | 2.96% |
| Rice bran wax | 5.70% |
| Colloidal oatmeal | 0.10% |
| Fish gelatin | 0.10% |
| Total | 100.00% |

PUFA Ratio: 1.00
Sum Omega3: 1.00
Penetration Ratio: 8.84

In Example 6, K7i, the product was whipped for 15 seconds @“8”, but otherwise followed the Example 5 processing. As made viscosity was 193,000 cp; as made specific gravity was 0.825. The product was whipped after aging. Aged viscosity was 283,000 cp; whipped specific gravity was 0.791.

Both products failed 40° C./15 hour thermal abuse test.

### Explanation of EXAMPLE 6

Free fatty acid destabilizes these nanoparticles. The heat abuse test drives off whipped-in gas. High shear destabilizes nano-liquid-capsules.

### Example 7 (Counter Example)

The following composition is prepared as described above:
Omeza Lotion K7j
   OTC Skin Protectant Lotion

| | |
|---|---|
| MCT oil | 69.43% |
| Monolaurin | 10.00% |
| Cetyl esters NF | 5.84% |
| Virgin coconut oil | 0.40% |
| Coconut FFA | 0.20% |
| Ascorbyl palmitate | 0.50% |
| Hemp oil | 2.41% |
| Cod liver oil | 2.36% |
| RBD palm oil | 2.96% |
| Rice bran wax | 5.70% |
| Colloidal oatmeal | 0.10% |
| Squalene/Vitamin E | 0.10% |
| Total | 100.00% |

PUFA Ratio: 1.00
Sum Omega3: 1.00
Penetration Ratio: 8.74
K7j failed the 40° C./15 hour thermal abuse test.

### Example 8 (Counter Example)

K7l is prepared is prepared as described herein. It has the following composition:
Omeza Lotion K7l
OTC Skin Protectant Lotion

| | |
|---|---|
| MCT oil | 69.70% |
| Monolaurin | 10.00% |
| Cetyl esters NF | 5.84% |
| Virgin coconut oil | 0.36% |
| Coconut FFA | 0.07% |
| Ascorbyl palmitate | 0.50% |
| Hemp oil | 2.41% |
| Cod liver oil | 2.36% |
| RBD palm oil | 3.00% |
| Rice bran wax | 5.66% |
| Colloidal oatmeal | 0.10% |
| Total | 100.00% |

PUFA Ratio: 1.00
Sum Omega3: 1.00
Penetration Ratio: 8.87

K7l was stable in the 40° C./15 hour thermal abuse test. Aged viscosity was 435,000 cp; aged specific gravity was 0.912. Cooling the composition after 125° F., stirring was reduced to “2”. Exotherms #2, 3 & 4 were observed for the first time. Coconut FFA was reduced.

### Explanation of EXAMPLES 7 and 8

Example 8 reduced the stirring rate during Exotherms #2, 3 & 4 to allow their freezing in distinct individual layers, leading to their discovery.

The strengthening of the nanoparticle skin by reducing FFA (from Example 7 @0.20% to example 8 @0.07%) was just enough to prevent leakage during 40° C./15 hour thermal abuse test.

### Example 9

K7p in this example was processed differently than in Example 1. It has the following composition:
OMEZA LOTION K7P

| | |
|---|---|
| MCT oil | 69.70% |
| Monolaurin | 10.00% |
| Cetyl esters NF | 5.84% |
| Ascorbyl palmitate | 0.50% |
| Hemp oil | 2.41% |
| Cod liver oil | 2.36% |
| RBD palm oil | 2.78% |
| Rice bran wax | 5.71% |
| Squalene/Vitamin E | 0.20% |
| Colloidal oatmeal | 0.10% |
| Coconut FFA | 0.05% |
| Virgin coconut oil | 0.35% |
| Total | 100.00% |

PUFA Ratio: 1.00
Sum Omega3: 1.00
Penetration Ratio: 8.91

This is the same composition as described in Example 4. In Example 9, K7p is processed differently than in Example 4. The hot fluid was stirred @“4” (wire whisk) until Exotherm #1 was complete (95° F.). Thereafter mixing was reduced to “stir” until Exotherms #2, 3 & 4 (85° F.) were complete. Then the mix was allowed to cool quiescently to 75° F. overnight. Part of the aged mix was undisturbed; part of the aged, cold mix was stirred 15 seconds (@“stir”). Samples of both were subjected to the thermal abuse test (40° C./15 hours) and examined for floating oil and/or sinking oil.

Table 2 summarizes the ambient temperature results for ambient specific gravity, viscosity, and leakage after heat abuse.

TABLE 2

| | Unstirred | Stirred 15 Seconds |
|---|---|---|
| Specific Gravity | 0.881 | 0.874 |
| Viscosity | 419,000 | 99,400 |
| Bottom Leakage | None | Slight |
| Top Leakage | None | Large |

### Explanation of EXAMPLE 9

In Example 4, when Cetyl Esters NF were increased, viscosity declined and specific gravity increased, the opposite of what was expected. In Example 9, it was determined that a minimal amount of ambient temperature shear caused a dramatic drop in viscosity and a slight reduction in specific gravity. Minimal shear also caused K7p to leak fluid to the bottom of the container and simultaneously the top of the container. MCT oil has a specific gravity of 0.931, greater than the 0.881 of unstirred K7p. It is unexpected that higher density fluid rises above a lighter mixture.

As described in Example 4, cetyl palmitate and cetyl myristate sequentially plate out on the underside of the monolaurin skin during slow cooling. After all the waxes melt and form a single miscible fluid (with colloidal oatmeal as an entrained solid), the mixture cools (modest shear) through Eutectic #1 (ascorbyl palmitate crystallization) and Eutectic #2 (rice bran wax (RBW) crystallization). Further cooling goes through Exotherm #1 (monolaurin forms nanoparticles (NLC) around the cargo (colloidal oatmeal and liquid lipids).

Further cooling (minimum shear) allows higher molecular weight wax ester (cetyl stearate—Exotherm #2) to form a thin solid film within the underside confines of each nanoparticle. Crystalized cetyl stearate increases the internal volume of each nanoparticle in equilibrium with the monolaurin circumferential compressive force (surface tension).

Further slow cooling from the outside of each nanoparticle allows time for first cetyl palmitate (Exotherm #3) and then cetyl myristate (Exotherm #4) to plate out on the underside of the monolaurin skin surrounding each nanoparticle. It is believed, without wishing to be bound, that the plated-out esters (“mortar”) strengthen the monolaurin skin by enveloping the monolaurin aliphatic tails (“rebar”). Pressure increases within each nano particle (surface tension “in;” volume increase “out”). As the nanoparticles pack together, the normal spherical structure is distorted by internal pressure, reducing interstitial pore volume, and increasing density as the volume of Cetyl Esters NF increases as the cetyl esters transition from high density liquid to lower density solids (Example 4). The low-density rice bran wax crystals are produced prior to the monolaurin sphere formation and thus float away from the forming spheres. Only at much lower temperature (during quiescent cooling) do the rice bran wax crystals precipitate onto the monolaurin outer surface.

Unexpectedly, when the viscous mass is disturbed by gentle stirring, the distorted spheres are released from steric binding with adjacent distorted spheres. Internal pressure “reflates” the distorted sphere, returning the individual distorted nanoparticle to a normal spherical shape and slightly reducing specific gravity (by allowing interstitial volume to be recovered as total volume increases). Normal spheres then roll over one another easily; viscosity is reduced.

With lower levels of Cetyl Esters NF, the reinforced skin strength is not sufficient to contain crystals and liquid lipids; some liquids leak out. The leakage is in all directions; some goes to the bottom of the container; some is squirted up to the top; some is captured in the interstitial volume.

Practically, K7p (5.84% Cetyl Esters NF) was replaced by K7r (8% Cetyl Esters NF (with a corresponding reduction in MCT—Example 11)). This eliminated the potential leakage and lowered viscosity.

### Example 10

K7r is prepared in accordance with the procedure herein in EXAMPLE 1. It has the following composition:
Omeza Lotion K7r
OTC Skin Protectant Lotion

| | |
|---|---|
| MCT oil | 67.54% |
| Monolaurin | 10.00% |
| Cetyl Esters NF | 8.00% |
| Ascorbyl palmitate | 0.50% |
| Hemp oil | 2.41% |
| Cod liver oil | 2.36% |
| RBD palm oil | 2.78% |
| Rice bran wax | 5.71% |
| Squalene/Vitamin E | 0.20% |
| Coconut FFA | 0.05% |
| Virgin coconut oil | 0.35% |
| Colloidal oatmeal | 0.35% |
| Total | 100.00% |

PUFA Ratio: 1.00
Sum Omega3: 1.00
Penetration Ratio: 8.64

K7r has an aged viscosity of 235,000 cp and an aged specific gravity of 0.894. It is not stirred after cooling to ambient temperature. K7r is a thick crème that is applied smoothly to the skin. K7r contains 1% omega3 fatty acids. The ratio of monolaurin to Cetyl Esters NF is 1.25:1.00. K7r contains 0.05% coconut FFA. Coconut FFA makes the crème greasier feeling; coconut FFA makes the crème less stable. However, a small amount of coconut FFA makes an anhydrous viscous crème feel like an elegant oil/water emulsion during rub-in, something consumers are used to and prefer.

High levels of MCT (permeation enhancer) and low levels of vegetable oil drive the vegetable oils through the skin (and the hair follicles) in 15 seconds. The “cargo” is deposited on the top of the stratum corneum. The monolaurin/ester structure phase-inverts. The monolaurin head group is attracted to the wet skin, exposing the now-flipped wax esters to the outside environment.

Consumers describe the effect of the phase inversion as a “silky smooth,” cosmetically elegant finish.

The wax esters trap RBW crystals and other cargo between the stratum corneum and the environment.

The fate of the cargo depends on the specifics of the cargo. A lipophilic API may traverse the stratum corneum directly or pass through the hair follicle into the dermis. A hydrophilic API may slowly become soluble and traverse the stratum corneum via the sweat glands. Other active cargo materials may remain trapped between the stratum corneum and the wax ester film.

The rice bran wax/wax ester/monolaurin film forms a semi-occlusive barrier over the skin. TEWL is reduced significantly, indirectly hydrating the skin. Oxygen is blocked from attacking unabsorbed unsaturated fats and the user does not smell off odors.

Scarring is reduced. The body responds to external insults by sending signals that then elicits a biological response. When the body senses a loss of barrier function (e. g. a burn, cut, scrape, incision, chronic wound, other skin conditions), it sends a signal that requests help to regain the normal barrier function. This help arrives as raw, avascular collagen without hair follicles, sweat glands or blood flow capillaries. The breach is stopped, and a scar forms. When the barrier is reestablished, collagenase enzymes attack the temporary scar collagen and remodel some of it into a stronger collagen. There is always a residual scar that most users dislike.

K7r creates a barrier after the wax ester/monolaurin phase inversion. The body does not sense a loss in barrier function; the body does not send a distress signal calling for additional scarring collagen; any scarring is minimal. K7r does not reduce scarring directly; K7r reduces the need for a collagen deposition barrier, thus indirectly reducing scarring.

K7r is allowed to cool to ambient temperature (from 85° F. to 75° F.) in its now-quiescent mixing/cooling bowl and then transferred to a 200 liter drum. A viscous fluid emptying system uses an extruder screw to gently pull K7r from the drum and feed it to a metering extruder that sends 30 ml into an oxygen barrier, flexible, multi-use package. K7r is packed into packages without shear.

The flexible “airless” package, e.g., a “tottle” (bottle that looks like a tube) uses a controlled dose metering pump (0.2 ml/stroke) to dispense K7r. The entire system is virtually shear-free to avoid premature rupture of the nanoparticles.

### Example 11 (Counter Example)

The composition of K7n is prepared in accordance with the procedure as in EXAMPLE 1 herein. It has the following composition:
Omeza Lotion K7n
OTC Skin Protectant Lotion

| | |
|---|---|
| MCT oil | 69.70% |
| Monolaurin | 10.71% |
| Cetyl esters NF | 5.39% |
| Virgin coconut oil | 0.40% |
| Ascorbyl palmitate | 0.50% |
| Hemp oil | 2.41% |
| Cod liver oil | 2.36% |
| RBD palm oil | 2.83% |
| Rice bran wax | 5.40% |
| Colloidal oatmeal | 0.10% |
| Squalene/Vitamin E | 0.20% |
| Total | 100.00% |

PUFA Ratio: 1.00
Sum Omega3: 1.00
Penetration Ratio: 8.81

K7n is a formula without coconut FFA. Virgin coconut oil was added after Exotherm #1 (nanosphere formation) was complete. This puts the coconut oil on the outside of the nanosphere, not on the inside.

K7n did not pass the 40° C./15 hour stress test. K7n was too greasy on rub-in.

### Example 12

K7s has the following composition:
Omeza Lotion K7s
OTC Skin Protectant Lotion

| | |
|---|---|
| MCT oil | 68.03% |
| Monolaurin | 10.00% |
| Cetyl Esters NF | 5.84% |
| Virgin coconut oil | 0.40% |
| Coconut FFA | 0.20% |
| Ascorbyl palmitate | 0.50% |
| Hemp oil | 2.41% |
| Cod liver oil | 2.36% |
| RBD palm oil | 2.96% |
| Rice bran wax | 7.00% |
| Colloidal oatmeal | 0.10% |
| Squalene/Vitamin E | 0.20% |
| Total | 100.00% |

PUFA Ratio: 1.00
Sum Omega3: 1.00
Penetration Ratio: 8.46

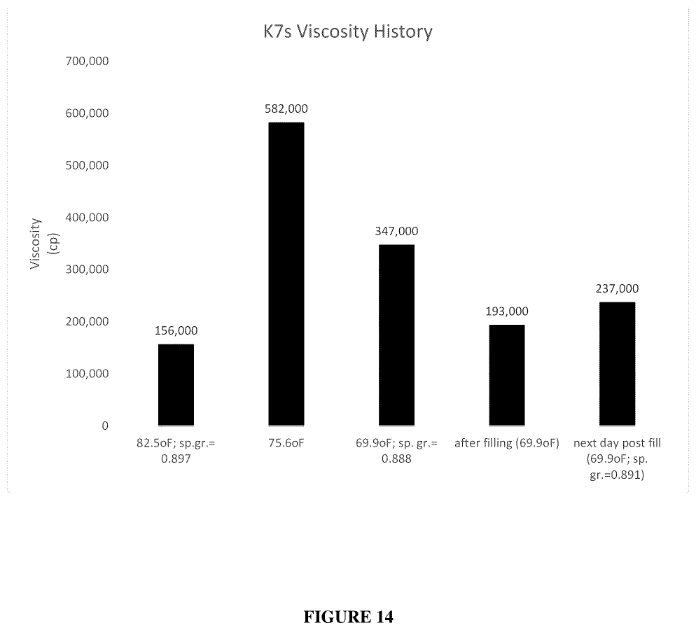

Fats, waxes, and fatty acids are melted together until the melt is clear (182° F.). After the melting step, all of the other steps below are conducted at room temperature. The hot melt is transferred to a 7 quart Kitchen Aid mixing bowl. The contents of the bowl are mixed at the lowest mixer setting identified as “Stir” (lowest mixing setting, 100 rpm) with standard mixing attachment (“flat beater” and/or “dough hook”), as the mixture is cooled until T=140° F. The floating grease that flies out of the mixture is scraped off the walls and repositioned into the liquid in the mixing bowl. The mixing in the bowl is continued at the lowest possible setting “Stir” until the temperature reaches 125° F. Colloidal oatmeal is added, and the resulting mixture is mixed at the lowest possible setting. Stirring is continued until the temperature reaches 90° F. The mixture is allowed to stand at room temperature The viscosity is measured at 82.5° F., the specific gravity is measured, and the viscosity is measured a second time at 75.5° F. The mixture is allowed to stand quiescent overnight unrefrigerated. The viscosity is measured again (@69.9° F.) and the specific gravity is measured. The mixture is loaded into a “turkey baster” syringe and the contents of the syringe are extruded into a beaker to simulate filling into tubes. The contents of the beaker are allowed to stand overnight. The next day, the viscosity is measured again (@69.9° F.), and the specific gravity is also measured. The data is shown graphically in FIG. 14.

### Explanation of EXAMPLE 12

EXAMPLE 12 was made with the lowest mixing speed (“stir”, beater attachment) on a standard Kitchen Aid mixer. The objective was to eliminate gas incorporation during cooling. Gas incorporation is a root cause reason for phase instability.

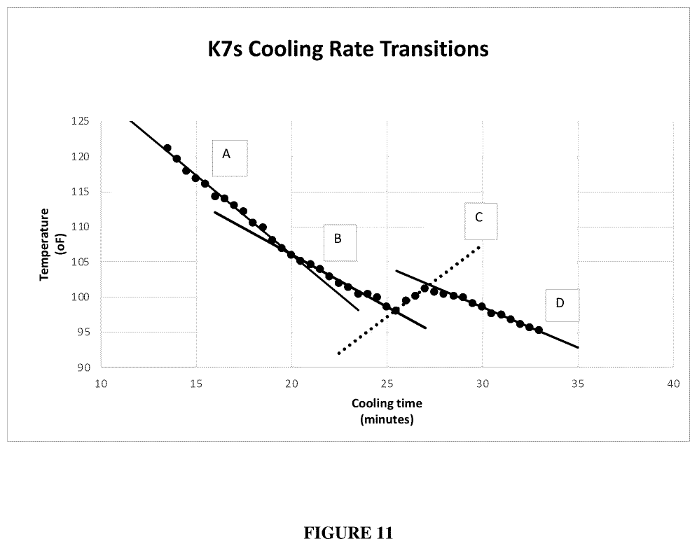

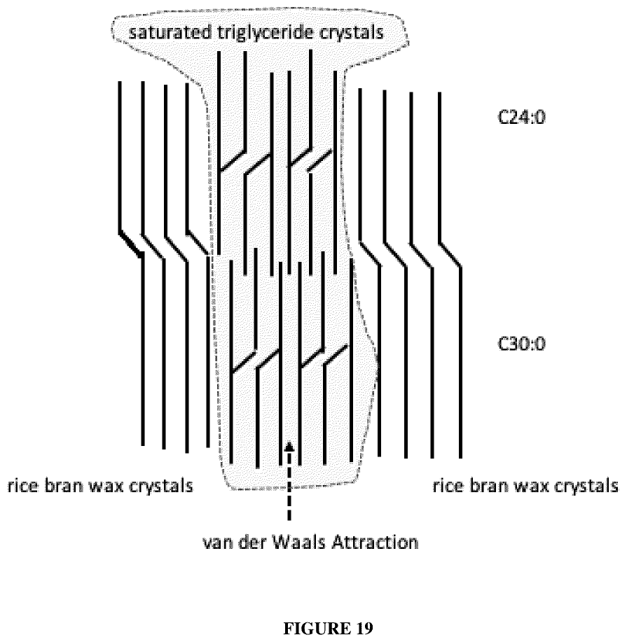

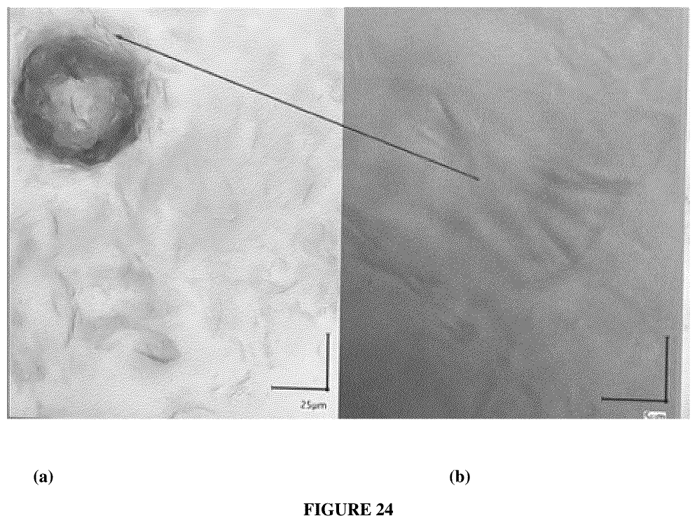

K7s goes through 9 documented cooling rate transitions during low shear cooling.
1. Rice bran wax precipitates on the cold sides of the mixing bowl as a hard wax film
2. Ascorbyl palmitate freezes, making the fluid cloudy.
3. Rice bran wax film is reincorporated in the melt and crystallizes on ascorbyl palmitate nuclei.
a. Viscosity increases, cooling rate declines.
4. Colloidal oatmeal is added.
a. Apparent cooling rate momentarily increases as cold oatmeal is added to hot melt.
b. Thereafter, the cooling rate declines as oatmeal absorbs fat and increases viscosity.
5. Tripalmitin, PPP (C16:0×3) freezes at the intersection of best fit line A and best fit line B (106° F., as shown in FIG. 11).
a. Viscosity increases as shown by the reduction in the slope of the best fit line from A to B.
b. The PPP solid grease commingles with the RBW/ascorbyl palmitate crystals, creating a structure analogous to “rebar and mortar.”
6. Monolaurin (nonionic surfactant) forms nano-liquid-capsules (NLC) around oatmeal, and remaining liquids.
a. The head group is to the exterior; the aliphatic tail is towards the interior.
b. Heavy solid cargo (e.g., colloidal oatmeal) is entrained in the encapsulated liquid with gentle mixing and becomes entrained in the encapsulated liquids.
7. Monolaurin freezes, immobilizing the nano-liquid-capsules.
8. Encapsulated cetyl stearate freezes around the aliphatic monolaurin tails, coating the inside of monolaurin circumferential skin.
9. Encapsulated cetyl palmitate freezes as a second interior layer over the first cetyl stearate layer.
10. Encapsulated cetyl myristate freezes as a third interior layer over the second layer cetyl palmitate layer; the three layers together help stabilize the nano-liquid-capsule.
11. Encapsulated, formerly liquid saturated fats (C12:0 & C14:0) freeze @-76-77° F. during quiescent cooling, further stabilizing each nano-liquid-capsule:
a. Viscosity increases.
b. Specific freezing of unsaturated fats (PUFA & C18:1) is not observed.
12. After the saturated fats freeze, the specific gravity of the composition decreases as amorphous liquids become higher specific volume, structured solids.
a. It is believed, without wishing to be bound, that this slight increase in volume distorts nano-liquid-capsule “spheres,” increasing viscosity dramatically as steric hindrance between individual nano-liquid-capsules increases.
b. The RBW/ascorbyl palmitate crystals (described in step 3 above) have an inherent lower density than the liquid melt (prior to Exotherm #1).
i. RBW/ascorbyl palmitate crystals float outside the NLC spheres during gentle mixing.
ii. When mixing stops and quiescent cooling begins, these crystals precipitate on the outside of each sphere and form a “rebar coating with PPP mortar” around each sphere (FIG. 24).
1. There is an NLC strengthening effect caused by the interior three layers of wax, the monolaurin layer and the RBW/ascorbyl palmitate/PPP outer layer. The strength is great enough to prevent leakage during filling, storage, and distribution, but weak enough to rupture when rubbed into skin.
c. As illustrated in FIG. 19, saturated fat crystals (e.g., PPP) plate out on pre-existing rice bran wax crystals.
i. Rice bran wax forms “dog-leg” crystals.
ii. Saturated triglyceride crystals form pairs of “stacked chairs” (or “tuning forks”)
iii. To visualize the phenomenon, the rice bran wax crystals are considered like the “weights” on a barbell; the saturated fat crystals are considered the “bar” joining two weights. With macro numbers of barbells, structures are formed that capture and sequester liquid fats. A meta stable system is formed with macro viscosity>400,000 cp.
iv. The triglyceride crystals are held together by weak van der Waals forces.
1. It is believed that these bonds are broken by shear, leading to sharp reductions in macro viscosity when quiescent composition is transferred to a second container or pumped into a consumer package.
13. When distorted nano-liquid-capsules are forcefully pushed through a filling nozzle and then released, nano-liquid-capsules revert to their lowest energy configuration (spheres) and viscosity is reduced.
14. With an overnight “rest” (analogous to pie crust making), encapsulated solid fats reestablish internal bonds; viscosity increases and is then stable.

FIG. 14 charts this viscosity history:
1. Viscosity is low (156,000 cp) before saturated fat solidification.
2. Viscosity almost triples (582,000 cp) as saturated fat congeals.
3. Viscosity falls −40% (347,000 cp) as fats reorient themselves during an overnight rest.
4. When the cooled former melt is extruded through a nozzle, viscosity (193,000 cp) declines another −40%.
5. Finally, viscosity increases +23% (237,000 cp) after a second rest allows the filled system to achieve its final equilibrium.

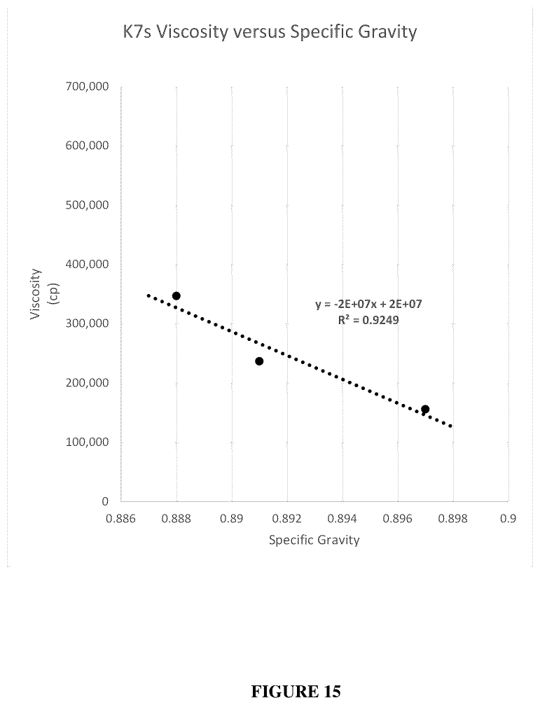

FIG. 15 shows that gas-free viscosity and % solids have a cause-and-effect relationship. When specific gravity is low, viscosity is high (R²=0.9249). That is, when amorphous liquids become structured, higher volume solids, specific gravity declines (volume increases) and viscosity increases. The distortions are released; the nano-liquid-capsules revert to their native lowest energy shape and viscosity drops.

K7s was subjected to the 40° C./15 hour abuse testing and was able to perform well and still rupture pleasantly on the skin.

Without wishing to be bound, it is believed that when the nanoparticle ruptures during rub-in, the contained oils, waxes and cargo spill onto the skin surface and are spread out. The released surfactant skin, with an ester-coated tail, inverts on the skin surface; the surfactant head is attracted to the moisture-rich stratum corneum. The ester-coated surfactant tail now faces the user. The user perceives a silky-smooth finish, if the last to precipitate ester is that ester with the silky-smooth finish (cetyl myristate).

When applied to the skin of a professional tester, the sensation felt by the professional tester was described as follows:
1. It is silky right up to the line (of being) greasy without going over the line.
2. Right level of sheen
3. (There is) a sensation of just right amount (remaining) on the skin.
4. It absorbs well.
5. There are no oily fingerprints (when the applying hand touches a hard surface).

Other testers describe: the compositions herein as
1. Pore size reduction on face with extended use.
2. Cosmetics are easier to apply.
3. Smoother skin

This composition of the present disclosure is a low oil/high MCT gel that is cosmetically elegant and simultaneously is a vehicle for delivering all kinds of different cargo to the skin surface.

Phase Transitions with K7s

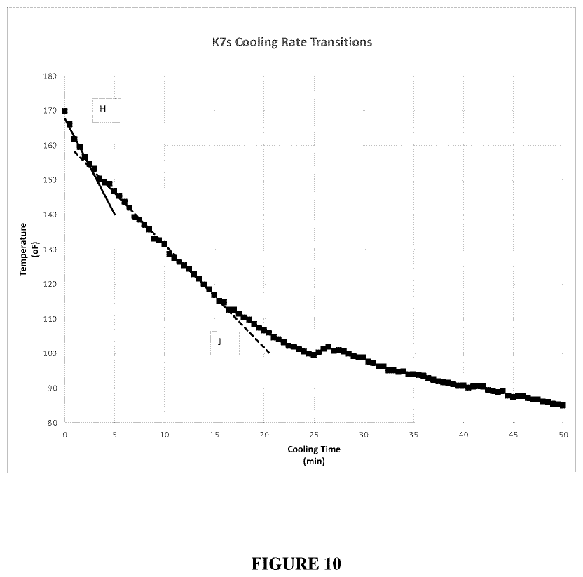

FIG. 10 is a plot of the cooling curve for K7s. Data show the phase transitions that combine Eutectic #1 with Eutectic #2 (the intersection of the two best fit lines, H & J).

Each time something solidifies during sheared cooling, the intrinsic viscosity of the mix increases and the rate of cooling declines.

For example, there is a 30% reduction in cooling rate (change in slope of the best fit lines) from A to B (FIG. 11).

The A-to-B transition marks the PPP freezing point. The B-to-C transition marks the exothermic formation of nano-liquid-capsules as the frozen monolaurin orients itself as the “skin” surrounding each nano-liquid-capsule to capture the cargo. The C-to-D transition marks continued cooling.

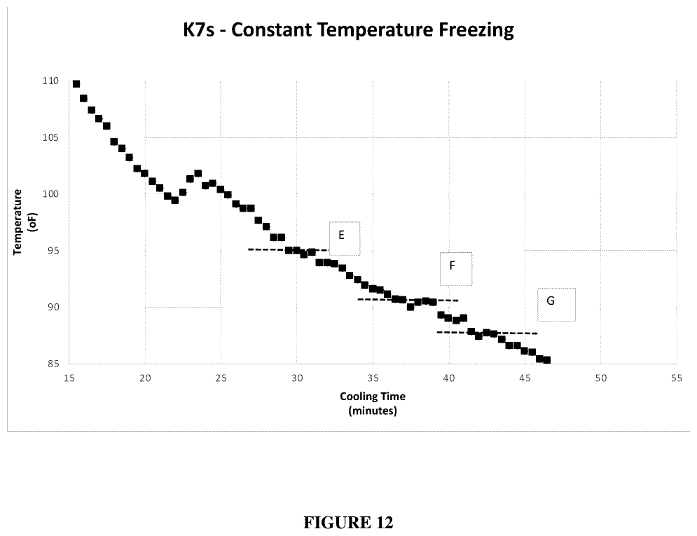

FIG. 12 is a continuation of the FIG. 11 data set. After the C-to-D transition, the mix continues to cool. At E, the temperature remains constant as the first-to-freeze cetyl ester solidifies. At F, the temperature is constant as the second-to-freeze cetyl ester solidifies. At G, the third cetyl ester freezes. This temperature response is consistent with well-known phase transition constant temperature tie lines.

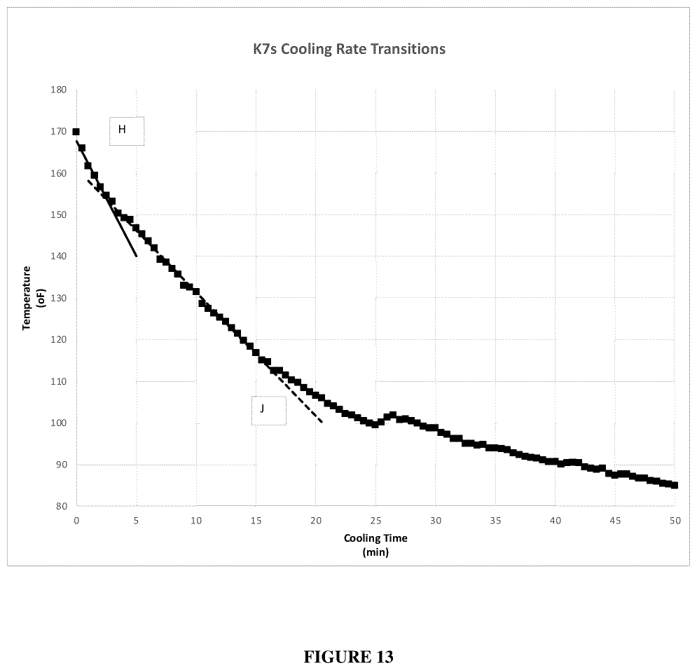

FIG. 13 is also a continuation of the FIG. 11 data set, except it highlights the high temperature transitions. At the H-to-J transition, ascorbyl palmitate freezes. During the J cooling, something unexpected occurs with rice bran wax. Rice bran wax has a higher melting point than ascorbyl palmitate. Rice bran wax plates out on the cooling surface during the transfer of hot fluid from the melting pot to the cooling pot. After scraping the vessel wall, plated wax “melts” (dissolves in the oil as a supersaturated solute) and begins to precipitate concurrently with ascorbyl palmitate at the H-to-J intersection.

In Example 10, K7r, Eutectic #1 and Eutectic #2 are separated because colloidal oatmeal is in the mix from the beginning. Ascorbyl palmitate precipitates @Eutectic #1. Ascorbyl palmitate nuclei compete with colloidal oatmeal “nuclei” for precipitating RBW. This produces two Eutectics. By simply adding solid cargo after RBW precipitates on ascorbyl palmitate nuclei, there is no competition for RBW crystals and all RBW crystalizes on the ascorbyl palmitate nuclei. This makes all the RBW crystals substantially uniform (average length ˜9 microns (FIG. 24)) and unchanging in size.

Conversely, when liquid cetyl esters crystalize inside each nano-liquid-capsule of Example 12, the enclosed volume changes as a function of cetyl ester freezing (volume increases) and thawing (volume decreases).

The solution to increase stability was to reduce cetyl esters (reduces internal volume variation during distribution), to increase RBW slightly, and to add particulate cargo after Eutectic #1 is complete (˜125° F.).

In the composition of the present disclosure, ascorbyl palmitate forms nuclei for rice bran wax nanocrystal formation, and the rice bran wax crystals, in turn, are nuclei for saturated fat (PPP) crystals (often described as “stacked chairs”). In preparing the compositions described herein, colloidal oatmeal addition is withheld until after the ascorbyl palmitate (AP) and rice bran wax (RBW) crystallization is complete, and crystal integrity of the RBW crystals and separately crystal integrity of the saturated fat crystals is improved. The mixture is phase stable during storage and distribution. TEWL (Trans Epidermal Water Loss), the amount of water that passively evaporates through skin to the external environment due to water vapor pressure gradient, is reduced.

Without wishing to be bound, it is believed that nano crystalline structures are formed by sequential layering:
1. Ascorbyl palmitate precipitates forming crystal nuclei.
2. Rice bran wax precipitates on the AP nuclei, forming nanocrystals.
3. Tripalmitin (PPP) precipitates on the nanocrystals, forming a complex crystal structure.
4. Monolaurin surfactant forms lower free energy nano-liquid-capsule spheres, encapsulating liquid fats, liquid wax, and fatty esters.
5. Monolaurin freezes, strengthening the spherical structure of the nano-liquid-capsule.
6. The RBW crystals (reinforced with solid PPP) are low density and float out and away from the spheres, eventually settling (Pickering Effect) on the sphere surface when mixing is terminated.
7. Colloidal oatmeal has higher density (>1.0) than liquid fats (sp. g.=0.91) and sinks (without mixing). If mixed gently, the oatmeal stays suspended and is encapsulated in the monolaurin sphere during sphere formation.
8. Three cetyl esters (cetyl stearate, cetyl palmitate, cetyl myristate) freeze in sequence under and around the aliphatic monolaurin tails, trapping the colloidal oatmeal inside the monolaurin “skin.”
9. Agitation is stopped @93° F., cooling progresses quiescently to ambient.
10. The freezing internal fats increase internal volume of each sphere; the spherical structure is mortared-in by the Pickering effect RBW/PPP crystals on the outer sphere surface.
11. Saturated fats>C10:0 (70% C16:0) precipitate and capture liquid lipids (oleogel) in each quiescent nano-liquid-capsule, increasing viscosity to >400,000 cp.
a. Natural palm oil is a mixture of fats (PPP, POP, PPO, OOP, 000) (where P=C16:0 and O=C18:1). The palm fats with two or more “Os” do not freeze at 70° F.
12. When product is disturbed (e.g., by pumping) the previously oriented, asymmetric nano-liquid-capsules become randomly un-oriented and viscosity drops to 200,000 cp.
   This structure is schematically depicted in FIG. 16.

Without wishing to be bound, it is believed that the process inverts after rubbing into the skin as shown schematically in FIG. 6.
1. Rub-in fractures the nano-liquid-capsule structure
2. The 73% liquid “cargo” (MCT+liquid fats) are absorbed into the dermis.
3. The monolaurin surfactant inverts and binds to the hydrated stratum corneum along with the now-inverted trapped ester/oatmeal layers.
4. The previously encapsulated colloidal oatmeal and unabsorbed saturated fat/RBW wax (saturated fat is 70% C16:0) form a moisture and oxygen occlusive contiguous film over treated skin.
a. There is a 15 second greasy feeling while saturated fats migrate towards the skin surface and the structure inverts.
b. The skin inhibits saturated fat absorption, forcing saturated fats and wax crystals to commingle in a flexible contact layer just above the skin surface.
5. Sequentially layered cetyl esters form a now-top layer over the wax/fat layer after monolaurin inversion.
6. The cetyl esters provide a cosmetically elegant finish over the greasy/waxy semi-occlusive layer. The inverted structure on the skin is depicted in FIG. 17.

Without wishing to be bound, the data herein suggests the following technical model:
1. Triglycerides, waxes, and lipophilic compounds melt and become a single phase.
2. Rice bran wax plates out on the cold wall during melt transfer as a floating grease.
3. During mixed cooling, heat is transferred through the wall of the mixing bowl to the atmosphere.
a. When plated-out RBW grease is scraped back into the hot lipophilic mix, RBW is solubilized into a super saturated solution.
4. Ascorbyl palmitate (AP) is a well-known, triglyceride crystal modifier.
5. When AP crystallizes (@ the H-to-J intersection), super saturated rice bran wax (RBW) concurrently crystallizes to form structured orthorhombic RBW crystals on AP nuclei.
a. It is hypothesized that the AP crystals function as nucleation sites for RBW “needles,” creating structured wax crystals.
6. Particulate cargo is optionally added after structuring the wax crystals to avoid interfering with crystal wax formation.
7. Monolaurin begins to solidify (FIG. 16).
8. Monolaurin “slush” forms into nano-liquid-capsules. Liquid triglycerides, liquid cetyl esters and particulate cargo are encapsulated by monolaurin.
a. This is an exothermic structure formation to a lower, more stable energy state (C).
i. Monolaurin/MCT nanoparticles are known in two-phase immiscible systems. They are unknown in single phase, anhydrous, collagen-free systems with rice bran wax.
9. Cooling continues.
10. Cetyl Esters NF is a mixture of cetyl esters (primarily C16:C18, C16:C16, C16:C14)
11. Each ester precipitates sequentially (FIG. 12 E, then F, then G), forming sequential layers of wax under/around the monolaurin aliphatic tail (coldest surface on each nano-liquid-capsule).
a. Cetyl ester G is furthest away from the monolaurin head group.
12. Mixing is terminated and the mix cools quiescently to ambient temperature.
13. Longer chain, saturated triglycerides (C12:0 to C24:0) coagulate as a complex greasy gel inside each nano-liquid-capsule, commingling with liquid fats (MCT and unsaturated fats), RBW crystals, and particulate cargo.
a. a. Some of these saturated triglycerides co-crystalize with the RBW crystal surfaces (FIG. 19)
14. Nano-liquid-capsules are not structurally robust.
a. Shear ruptures the particles.
i. High shear stirring causes leakage.
1. In a 40° C./15 hour stress test, modest hydraulic shear can rupture previously sheared, nano-liquid-capsules, releasing a sinking wax/oil mixture that solidifies on subsequent cooling.
b. The structure of internal, encapsulated solids affects stability.
i. With excess cetyl ester concentration, the volume increase from post-formation crystallization is sufficient to rupture the nano-liquid-capsule and extrude “floating” MCT (sp.gr=0.931) out and above the nano-liquid-capsules (sp. gr.=0.86).
1. Capillary forces transport leaked liquids to the surface of the composition.
15. The weakness of each nano-liquid-capsule is a material advantage for topically applied products.
a. The rub-in is gentle and immediate.
b. MCT is a penetration enhancer that drags unsaturated fats through the epidermis.
c. Particulate cargo and RBW crystals deposit on the skin surface.
i. Cargo fate depends on the nature of the cargo.
ii. “Hidden” RBW/saturated fat crystals are trapped between the stratum corneum and the nano-liquid-capsule skin fragments.
d. Ruptured, residual monolaurin skin aliphatic tails contain layered cetyl esters.
e. The monolaurin head group is attracted to the “wet” stratum corneum.
f. The monolaurin/layered cetyl ester skin fragments invert.
i. The head group (glycerol) faces the stratum corneum.
ii. The aliphatic, layered wax tails (C12:0+embedded, layered cetyl esters) face the “sky”.
g. The consumer senses:
i. Lotion-like rub-in (10-15 seconds) as the nano-liquid-capsule ruptures and the encapsulated oils absorb into the skin.

A non-greasy, silky-smooth residual as the nano-liquid-capsule skin inverts.

An intentionally meta stable structure is formed that is robust enough to tolerate commercial packaging, storage, and distribution for a minimum 24-month shelf life while simultaneously being weak enough to rupture when sheared during skin application. This balancing act is difficult, particularly when after skin absorption is complete the residual must be cosmetically elegant.

### Example 13

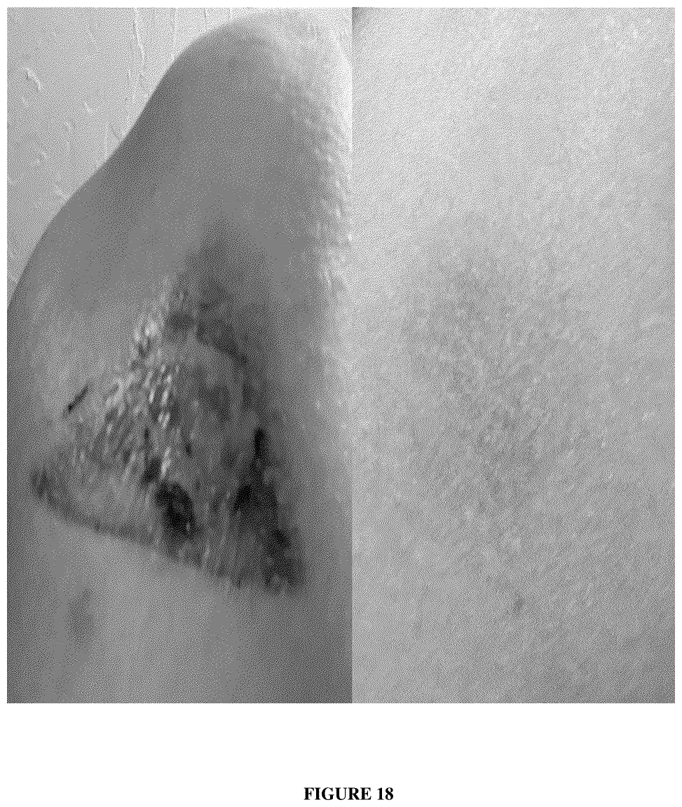

A female burned the back of her arm on a hot oven door. The left side of FIG. 18 is a photo depicting the burn. K7r was applied daily for 3.5 months. The photo on the right shows the burn area after treatment. As can be seen, the burn site shows no visible visual evidence of the burn. The burn has no scarring; the skin texture is normal.

K7r is an excellent product, but it is missing one important feature. Scarring is reduced and the skin is more resistant to pathogen growth if the skin pH is slightly acidic (i.e., a robust acid mantle). Anhydrous lipid-rich compositions do not have free protons per se because there is not any water to ionize the fatty acids. But attempts to increase the FFA failed because the composition was unstable.

Example 14 addresses the slightly acidic issue by replacing coconut FFA (K7s) with caprylic acid (K7LH5), increasing FFA by 78.7% (see Explanation of Example 14). In Example 14, the processing was changed slightly to allow sphere formation to take place in the absence of shear. This simple change allowed more acid incorporation without destabilizing the system.

### Example 14

Unlike many of the compositions above, this composition does not contain coconut free fatty acid (a mixture of C8, C10, C12 and C14 FFAs). Instead, it contains caprylic acid (C8). In addition, an optional fragrance is added to help offset any residual odor.

The composition is as follows:
Remodeling Cream—K7LH5
OTC Skin Protectant Lotion

Capric/Caprylic

| | |
|---|---|
| Triglyceride | 68.00% |
| Monolaurin | 10.00% |
| Cetyl Esters NF | 5.84% |
| Ascorbyl Palmitate | 0.50% |
| Hemp Oil | 2.41% |
| Cod Liver Oil | 2.36% |
| RBD Palm Oil | 3.00% |
| Rice Bran Wax | 7.00% |
| Squalene/Vitamin E | 0.20% |
| Colloidal Oatmeal | 0.10% |
| Caprylic Acid | 0.23% |
| Coconut Oil | 0.33% |
| Fragrance B | 0.03% |
| Total | 100.00% |

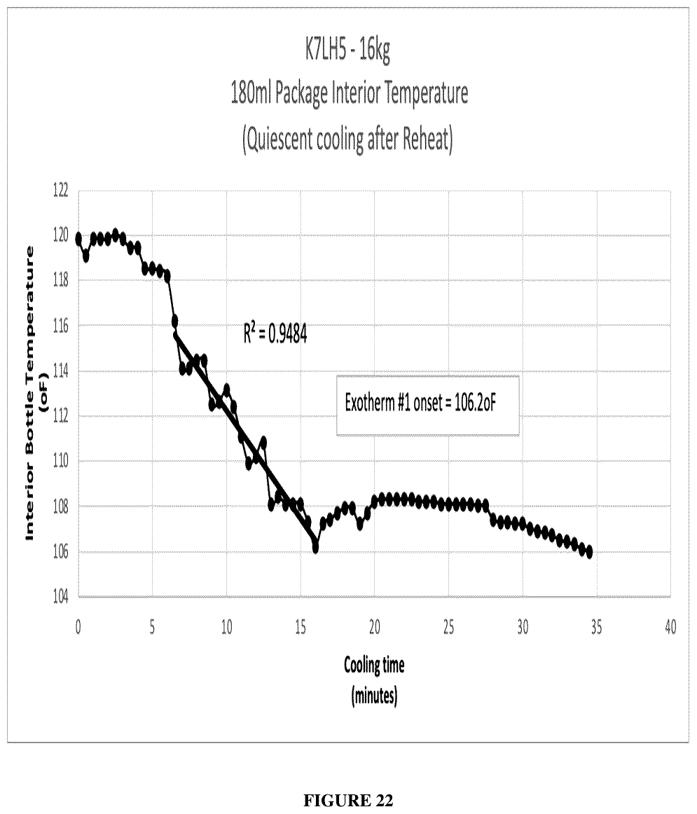

PUFA Ratio: 1.00
Sum Omega3: 1.00
Penetration Ratio; 8.74
   K7LH5 passes the 15 hour/40° C. stability abuse test.
   K7LH5 is made slightly differently than in prior examples:
1. All of the ingredients, except the fragrance and colloidal oatmeal are heated with mixing to 185° F. in a mixing vessel, @“stir” such as, in a 50 gallon heat/cool, agitated kettle.
2. The contents in the vessel are cooled with gentle stirring, for example at the lowest possible setting that maintains agitation of the contents of the mixing vessel until 125° F. If there is some precipitation formed on the walls of the vessel, the walls are wiped, with the contents added to the mixing vessel.
3. Colloidal oatmeal is added.
4. The mixing is continued until the temperature reaches 110° F.; then optional fragrance is added.
5. The mixture is pumped into a holding vessel, such as 1001 Muller drum (American International Container, Boonton NJ), a vessel designed to pump highly viscous gels, for quiescent cooling (i.e., with no mixing) to 75° F.
6. The stiff gel (from the holding vessel) thus formed is extruded as a stiff gel through a temperature-controlled, heat exchanger that holds product at 105-110° F.
a. The gel is broken, but the viscosity is 2,112-5,900 cp.
b. Specific gravity is 0.901.
7. There is no oatmeal separation when mixed at 3,000-5,000 cp. In other words, the composition is made sufficiently thick so that the colloidal oatmeal does not precipitate during quiescent cooling. In this way, the composition remains the same throughout the mixture. then on reheating with gentle mixing, the composition is consistent.
8. The consumer containers are filled with the product thus formed. For example, 170 ml of the product is placed into a Boston Round pump bottle (SR Packaging Taiwan).
9. The container is closed, such as by screwing in the Boston Round pump bottle pump closure.
10. The bottle with the contents therein are cooled to ambient temperature.
a. The product gels @106.2° F. (FIG. 22)
11. Aged viscosity in the bottle is 532,000 cp.
12. Viscosity of pumped K7LH5 is 205,000 cp after shearing through the bottle pump.
a. This viscosity will change slightly when winter/summer ambient conditions change product temperature.
13. The dispensing pump delivers 1 ml/squirt.

### Explanation of EXAMPLE 14

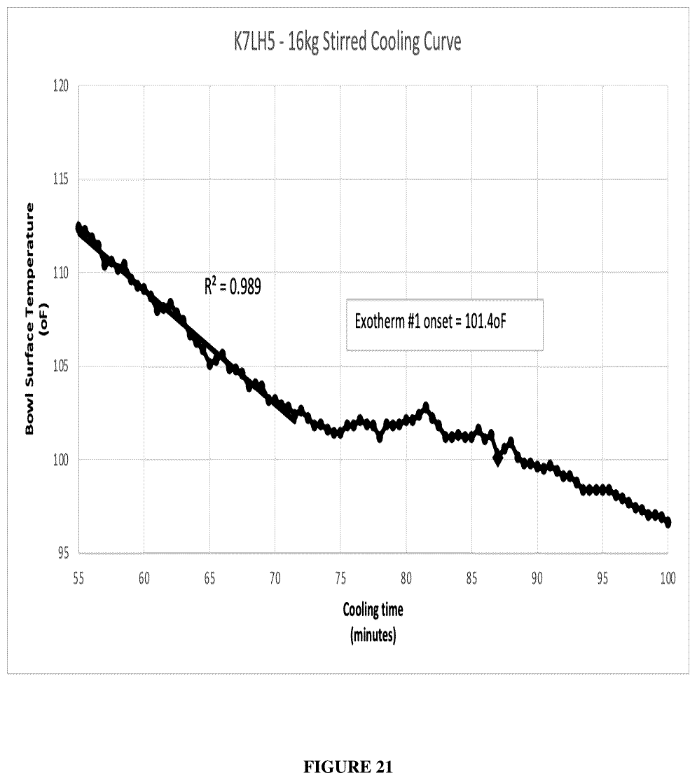

There is a difference between agitated cooling to ambient and quiescent cooling to ambient as shown in FIG. 21 and FIG. 22. Agitated cooling and quiescent cooling have similar temperature response curve shapes, but the monolaurin sphere formation takes place at about 5° F. higher temperature during quiescent cooling. This is seen as higher delivered viscosity out of the pumped bottle. Product testers prefer the higher viscosity product because it is less likely to run when applied to skin surfaces while standing; it rubs in rapidly, having lotion-like application (easy-to-apply), and is non-greasy, moisturizing, and cosmetically elegant. When rubbed into fresh, fully epithelialized wounds for 1-2 months daily, scarring is reduced; moreover, any residual scar has the same color as surrounding skin.

K7LH5 is positioned as a Remodeling Gel to minimize scarring and prevent recently closed wounds from dehiscing.

The FIG. 20 cooling curve for K7LH5 resembles the cooling curve for K7s (FIG. 13). What is different in the formulations is the substitution of caprylic acid for coconut free fatty acid and a nominal amount (0.03%) of alcohol-free fragrance.

The process of making K7LH5 is different. K7s was extruded cold into packages, resulting in shear-related viscosity loss. K7LH5 was reheated enough to break the monolaurin NLC structure, but not break the rice bran wax crystal or the tripalmitin (PPP) mortar structure and then pumped (as a slurry) into the final package. The gel forms quiescently in the package without shear.

Hot filling of products is a well-known procedure, with subsequent gelation in a final package (e.g., deodorant sticks).

Free fatty acid is typically removed from cosmetic compositions. But free fatty acid can buffer the skin's acid mantle pH, making the acid mantle a better antimicrobial barrier.

Caprylic acid (C8) is about 7.9% of coconut free fatty acid (used in K7s). On a mole basis, C8 is 12.22% of coconut free fatty acid. as shown by the following Table 3 of the composition of coconut free fatty acid:

TABLE 3

| | weight % | Molecular weight | mole % |
|---|---|---|---|
| C8:0 | 7.86% | 144.21 | 12.22% |
| C10:0 | 6.24% | 172.26 | 8.13% |
| C12:0 | 52.85% | 200.32 | 59.16% |
| C14:0 | 20.88% | 228.37 | 20.50% |

Thus, converting from coconut free fatty acid to caprylic acid (w/w) increases the molar acid content without increasing the weight percent of destabilizing FFA.

K7LH5 is 0.23% caprylic acid (0.15949 moles/100 g); K7s is 0.2% coconut fatty acid (0.0892031 moles/100 g). Thus, a major difference between K7LH5 and K7s is K7LH5 has more moles of acid (+78.7%).

But adding free fatty acid destabilizes the composition. Gelling in the final package without melting RBW needle crystals allowed adding more acid without destabilizing the gel.

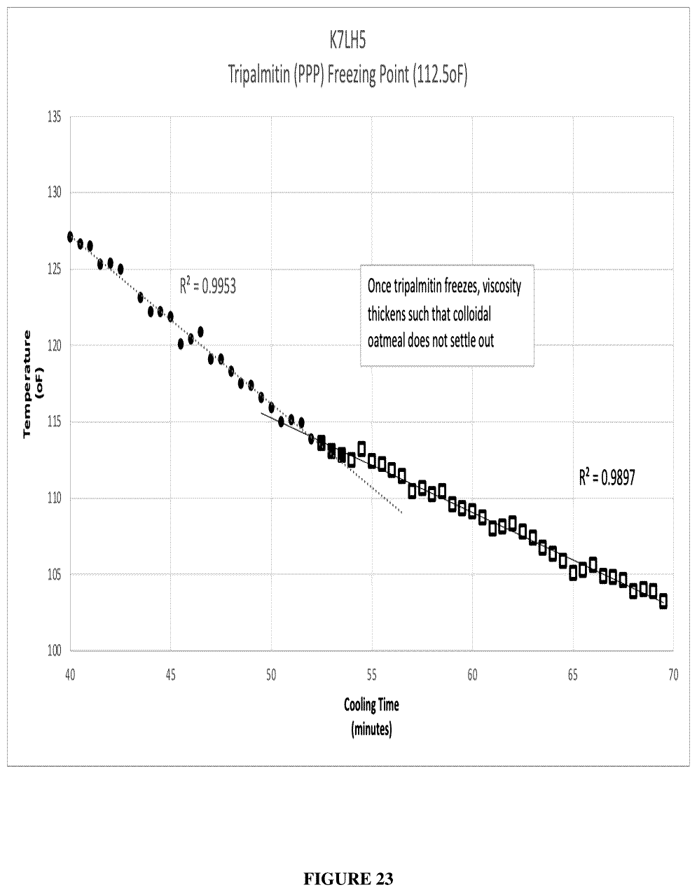

During processing, colloidal oatmeal settles rapidly in thin fluids. As shown in FIG. 23, the tripalmitin fraction of palm oil (PPP) freezes @112.5° F. with a consequent increase in viscosity.

It was determined experimentally, that terminating mixing @110° F.:
1. Kept colloidal oatmeal from precipitating.
2. Allowed quiescent formation of the monolaurin sphere structure.
3. Eliminated floating oil during 15 hour/40° C. stability testing.

More analysis was done on the monolaurin structure (FIG. 24). It was determined that RBW needle crystals are not inside the monolaurin structure but are on the outside surface (Pickering Effect). The RBW crystals are embedded in a gray grease, also on the outside of the monolaurin sphere.

Without wishing to be bound, it is believed that the observed structure can be explained:
1. RBW initial crystals are amorphous and freeze first on the “cold” mixing vessel surface.
2. Ascorbyl palmitate (AP) crystals form at ˜151° F.
a. Point A in FIG. 20
3. The AP seeds are nuclei for the transformation of RBW amorphous wax into needle like crystals, on average about 9 microns long (FIG. 24).
a. This occurs between points A and B in FIG. 20.
b. RBW needle crystals have a density<MCT (0.931) so they “float” outside the monolaurin spheres as the spheres form (Pickering Effect). In other words, crystals of low-density float in the liquid oil so that when the structure forms, the crystals are outside the structure.
4. Viscosity is unchanged from B to C in FIG. 20 until tripalmitin freezes (FIG. 23) @112.5° F.
a. The slope of the line is unchanged from B to C.
5. Nano-liquid-capsule (NLC) formation begins @point C in FIG. 20.
a. Viscosity continuously increases until Exotherm #1 (101.4° F., FIG. 21) is complete.
6. Monolaurin begins to form NLC spheres (FIG. 21 Exotherm #1)
a. The spheres encapsulate all the liquid (and denser “cargo;” e.g., colloidal oatmeal)
7. Once the NLC sphere formation is complete (point D in FIG. 20), cooling continues linearly (i.e., constant rate (slope of line remains constant).
8. But a series of liquid cetyl esters and liquid saturated (C≥12:0) fats freeze inside each sphere during post Exotherm #1 cooling.
a. As these crystals create structure, their volume increases (lowering density).
b. Surfactant surface tension creates an outside/in pressure that increases inversely with sphere diameter (i.e., pressure is inversely proportional to diameter).
c. Structured waxes and encapsulated saturated fats create an inside/out pressure as they organize into an organized, lower density crystal structure. In other words, surface active molecules create an inward pressure, when the spheres are formed, encapsulated liquids freeze and increase their specific volume. When the internal volume increases, the pressure is in-to-out.
i. Crystal formation forms structures that do not pack as tightly as amorphous liquids (i.e., less dense).
d. These two forces find an equilibrium when the product is formed.
9. Encapsulated triglyceride crystals (e.g., C12:0 & C14:0 from coconut oil) are sterically hindered from exiting the monolaurin sphere.
10. PPP crystals form a grease before NLC formation (Pickering RBW crystals with extruded “PPP mortar” (the gray “mud” seen in photographs (FIG. 24)).
a. Viscosity increases to >500,000 cp.
11. When the high viscosity gel is sheared during pumping from the consumer's bottle, some spheres rupture and some triglycerides are released.
a. Viscosity falls.
12. Alternately, sheared gel is reheated to a temperature above the monolaurin sphere forming temperature (onset of Exotherm #1) and below the temperature where RBW needle crystals and tripalmitin melt (Point C in FIG. 20).
13. Melt is pumped into user-packages where gelation reforms without shear.
a. The onset of Exotherm #1 increases by about 5° F. (FIG. 22).
b. This increase makes the reformed gel more stable and allows K7LH5 to withstand the 15 hr/40° C. stress test.
14. The improvement was so dramatic that the-substitution of caprylic acid for coconut FFA was feasible, increasing the low pH antimicrobial efficacy of the natural skin acid mantle without destabilizing the mixture.
a. Caprylic acid still tends to reduce stability, but a balance can be found experimentally between pH buffering and destabilizing the composition.
15. When the product is rubbed into the skin, MCT is released and penetrates the skin.
16. The monolaurin skin (with PPP mortar embedding external RBW wax crystals) inverts and binds to the skin (as surfactants are designed to do), forming a moisture occlusive wax layer that helps minimize moisture loss (reduced TEWL) (FIG. 6).
17. Caprylic acid helps buffer skin acid mantle pH, increasing the antimicrobial potential of native skin.
18. Colloidal oatmeal remains on the skin surface along with PPP/RBW crystals (“mortar/rebar”). The combination creates a silky-smooth, cosmetically elegant finish.
19. Without wishing to be bound, it is believed that scarring is reduced because the closed wound is well moisturized; the scar is soft from intradermal unsaturated fats, thus reducing stress on the scar tissue (a root cause of excessive scarring).
20. The lower pH, from more caprylic acid, encourages matrix metalloprotease enzymatic (MMP) activity.
a. MMPs breakdown the temporary collagen that closes the wound.
b. The body naturally replaces broken down weak collagen with stronger permanent collagen.
21. The optional alcohol-free fragrance, such as Fragrance B, is added to “confuse” the nose.
a. It is not a traditional perfume that adds a particular value-added odor, but rather a mixture of odors designed to “mis-identify” fish odors.
b. The fragrance covers any raw material odor and makes a barely perceptible combined skin scent that is imperceptible about 15 seconds after application.
c. Perfume is not mandatory but added optionally to match consumer expectations.
22. The net result is that well hydrated, low-stress, acidic, cosmetically elegant skin composition that encourages patient compliance while the body remodels weak collagen into strong collagen.
a. Any scar is almost invisible.

### Example 15

The composition of the composition of Example 15 is provided below:
Remodeling Cream—K7LH14
   OTC Skin Protectant Lotion

Capric/Caprylic

| | |
|---|---|
| Triglyceride | 70.00% |
| Monolaurin | 10.00% |
| Cetyl Esters NF | 5.84% |
| Ascorbyl Palmitate | 0.50% |
| Hemp Oil | 3.70% |
| Cod Liver Oil | 3.53% |
| Rice Bran Wax | 5.50% |
| Squalene/Vitamin E | 0.20% |
| Colloidal Oatmeal | 0.10% |
| Caprylic Acid | 0.25% |
| Coconut Oil | 0.35% |
| Fragrance B | 0.03% |
| Total | 100.00% |

PUFA Ratio: 1.00
sum Omega3: 1.5
Penetration Ratio: 9.5

Example 15 is prepared in accordance with the procedure of Example 14.

### Explanation of EXAMPLE 15

Example 15 and Example 14 are very similar except rice bran wax was reduced from 7% to 5.5%; hempseed oil and cod liver oil were increased slightly along with small changes to the amount of caprylic acid and coconut oil. Capric/caprylic triglyceride (MCT) was increased by 2% to make materials balance to 100%. Palm oil was eliminated.

Reducing RBW reduced viscosity; eliminating palm oil eliminated the inflection point @112.5° F. (FIG. 23). Eliminating this inflection point reduced viscosity also.

K7LH14 has an optimized viscosity that can be dispensed with a bottle pump and still retain cosmetic elegance, hydration, and skin suppleness.

### Example 16

The following is the composition of Example 16:
Remodeling Cream—K7LH17
OTC Skin Protectant Lotion

| | |
|---|---|
| Capric/Caprylic Tiglyceride | 70.00% |
| Monolaurin | 10.00% |
| Cetyl Esters NF | 5.84% |
| Ascorbyl Palmitate | 0.50% |
| Hemp Oil | 3.70% |
| Colloidal Oatmeal | 0.03% |
| Cod Liver Oil | 3.53% |
| Rice Bran Eax | 5.50% |
| Squalene/Vitamin E | 0.20% |
| Zinc Acetate | 0.08% |
| Caprylic Acid | 0.25% |
| Coconut Oil | 0.34% |
| Gragrance B | 0.03% |
| Total | 100.00% |

PUFA Ratio: 1.00
Sum Omega3: 1.5
Penetration Ratio: 9.51

Example 16, K7LH17 is prepared in accordance with the procedure of Example 15.

### Explanation of EXAMPLE 16

Example 16 is very similar to Example 15, K7LH14, except that colloidal oatmeal is reduced and zinc acetate is added as a new “cargo” ingredient. Colloidal collagen remains as an active ingredient according to the US FDA Skin Protectant monograph (>0.007 mg/l).

Zinc ions are important co-factors along with matrix metalloproteases (MMP). MMP are the enzymes that modify weak scar collagen during wound repair.

Zinc acetate is soluble in 40 g/100 g water at 25° C.; 67 g/100 g water at 100° C.; 3 g/100 g alcohol at 25° C. It is not soluble in oil. In Example 16, zinc acetate unexpectedly dissolved @0.08 g/100 g (@185° F.) in Example 16 oils.

During remodeling, MMP break down scar collagen. The body then replaces weak collagen with strong collagen as remodeling proceeds to completion.

The above preferred embodiments and examples were given to illustrate the scope and spirit of the present invention. These embodiments and examples will make apparent to those skilled in the art other embodiments and examples. The other embodiments and examples are within the contemplation of the present invention. Therefore, the present invention should be limited only by the amended claims.

What is claimed is:

1. A topical anhydrous composition, the topical anhydrous composition comprising:
medium-chain triglycerides (hereinafter “MCT”), monolaurin, cetyl esters, marine oil, vegetable oils having an omega 3 fatty acid content greater than 9 wt %, and rice bran wax;
wherein the topical composition comprises a viscosity ranging from about 50,000 cp to about 400,000 cp;
wherein the topical composition comprises a specific gravity ranging from about 0.83 to about 0.92; and
wherein the topical composition comprises a penetration ratio ranging from about 1.0 to about 11.0.

2. The topical composition of claim 1, further comprising ALA, SDA, EPA and DHA and a weight ratio of a sum of weights of (ALA+SDA)/sum of weights of (EPA and DHA) present in the composition ranging from about 0.5 to about 2.0.

3. The topical composition of claim 2, wherein the omega 3 fatty acid is present in the composition from about 0.5 to about 8 wt % of the composition.

4. The topical composition of claim 2, wherein MCT is present in the composition in an amount ranging from about 40 wt % to about 75 wt %, the monolaurin is present in an amount ranging from about 7 wt % to about 14 wt %; the cetyl esters are present in an amount ranging from about 4 wt % to about 12 wt %; the marine oil is present in an amount ranging from about 0.5 wt % to about 8 wt %; the vegetable oil having an omega 3 fatty acid content greater than 9 wt %, is present in an amount ranging from about 0.5 wt % to about 8 wt %; and the rice bran wax is present in an amount ranging from about 2 wt % to about 12 wt %; wherein the sum of the above-identified ingredients in the composition is no greater than 100 wt %.

5. The topical composition of claim 2, wherein the sum of a weight percentage of the MCT, the monolaurin, the cetyl esters, the marine oil, the vegetable oils having an omega 3 fatty acid content greater than 9 wt %, and the rice bran wax is about 75 wt % to about 100% of the composition.

6. The topical composition of claim 5, wherein the viscosity is about 150,000 cp to about 250,000 cp at 75° F. and 14.7 psig.

7. The topical composition of claim 6, wherein the specific gravity is about 0.85 to about 0.92 at 75° F. and 14.7 psig.

8. The topical composition of claim 7, wherein the vegetable oil having an omega3 fatty acid content greater than 9 wt % and the marine oil are present in a weight ratio of about 1:0.5 to about 2:1.

9. The topical composition of claim 8, wherein the marine oil is present in an amount from about 1 wt % to about 5 wt %.

10. The topical composition of claim 9, wherein the MCT is present in an amount from about 60 wt % to about 75 wt %.

11. The topical composition of claim 10 wherein the monolaurin is present in amounts from about 8 wt % to about 13 wt %.

12. The topical composition of claim 11, wherein the cetyl esters are present in an amount from about 5 wt % to about 8 wt %.

13. The topical composition of claim 12, wherein vegetable oil having an omega3 fatty acid content greater than 9 wt % is present in an amount from about 1 wt % to about 5 wt %.

14. The topical composition of claim 13, wherein rice bran wax is present in an amount from about 4 wt % to about 10 wt %.

15. The topical composition of claim 14, wherein vegetable oil having an omega 3 fatty acid content greater than 9 wt % is hemp oil.

16. The topical composition of claim 15, further comprising colloidal oatmeal, ascorbyl palmitate and coconut oil.

17. The topical composition of claim 16, wherein coconut oil is present in an amount from about 0.2 wt % to about 1 wt %.

18. The topical composition of claim 17, wherein colloidal oatmeal is present in an amount from about 0.01 wt % to about 2 wt %.

19. A topical composition, the topical composition comprising:
medium-chain triglycerides (hereinafter “MCT”) oil in about 68.00 wt %, monolaurin in about 10.00 wt %, Cetyl Esters in about 5.84 wt %, Virgin coconut oil in about 0.33 wt %, caprylic FFA in about 0.23 wt %, ascorbyl palmitate in about 0.50 wt %, hemp oil in about 2.41 wt %, cod liver oil in about 2.36 wt %, RBD palm oil in about 3.00 wt %, rice bran wax in about 7.00 wt %, colloidal oatmeal in about 0.10 wt %, and Squalene/Vitamin E in about 0.20 wt %.

20. A method of reducing the tendency of scarring from a wound, burn or skin condition, where the skin thereof is broken in a subject, the method comprising:
applying a therapeutic amount of a topical composition directly onto the wound, burn, or skin condition, the topical composition comprising:
MCT, monolaurin, cetyl esters, marine oil, vegetable oils having an omega 3 fatty acid content greater than 9 wt %, rice bran wax, said composition having a viscosity ranging from about 50,000 cp to about 400,000 cp and a specific gravity ranging from about 0.83 to about 0.92, and a penetration ratio ranging from about 1.0 to about 11.0; and
wherein the topical composition is fully granulated on the skin; and
wherein the topical composition is disposed on the skin for a predetermined amount of time to retard scaring, inhibit scarring, or both.
